(19) United States
(12) Patent Application Publication
(10) Pub. No.: US 2023/0285144 A1
(43) Pub. Date: Sep. 14, 2023

(54) MECHANICALLY EXPANDABLE PROSTHETIC DEVICE

(71) Applicant: Edwards Lifesciences Corporation, Irvine, CA (US)

(72) Inventors: Haim Yushtein, Netanya (IL); Anatoly Dvorsky, Haifa (IL); David Maimon, Atlit (IL); Noam Nir, Pardes-Hanna (IL); Alon Ben-Yosef, Ramot Menashe (IL)

(73) Assignee: Edwards Lifesciences Corporation, Irvine, CA (US)

(21) Appl. No.: 18/015,707

(22) PCT Filed: Jul. 8, 2021
(86) PCT No.: PCT/US2021/040789

§ 371 Date: Jan. 11, 2023

Related U.S. Application Data
(60) Provisional application No. 63/049,812, filed on Jul. 9, 2020.
(60) Provisional application No. 63/073,622, filed on Sep. 2, 2020.
(60) Provisional application No. 63/138,599, filed on Jan. 18, 2021.
(60) Provisional application No. 63/211,892, filed on Jun. 17, 2021.

(51) Int. Cl. A61F 2/24

(52) U.S. Cl. CPC A61F 2/2418; A61F 2/243; A61F 2/2439; A61F 2220/0091; A61F 2250/001; A61F 2250/0029

(57) ABSTRACT

A prosthetic device, such as a prosthetic heart valve, can include a frame that is radially expandable and compressible between a radially compressed state and a radially expanded state. In some examples, the prosthetic device can include locking mechanisms that are configured to hold the prosthetic device in a radially expanded state. In some examples, the locking mechanisms can be integrated into the frame of the prosthetic device.

### CROSS-REFERENCE TO RELATED APPLICATIONS

This application claims the benefit of U.S. Provisional Application No. 63/211,892 filed Jun. 17, 2021, U.S. Provisional Application No. 63/138,599 filed Jan. 18, 2021, U.S. Provisional Application No. 63/073,622 filed Sep. 2, 2020, and U.S. Provisional Application No. 63/049,812 filed Jul. 9, 2020, which are incorporated by reference herein in their entirety.

### FIELD

The present disclosure relates to implantable, mechanically expandable prosthetic devices, such as prosthetic heart valves, and to methods, assemblies, and apparatuses for delivering, expanding, implanting, and deploying such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various fully functional working diameters. Some mechanically expandable prosthetic heart valves can also be compressed after an initial expansion (e.g., for repositioning and/or retrieval).

Despite the recent advancements in percutaneous valve technology, there remains a need for improved transcatheter heart valves and delivery devices for such valves.

### SUMMARY

The present disclosure relates to implantable, mechanically expandable prosthetic devices, such as prosthetic heart valves, and to methods, assemblies, and apparatuses for delivering, expanding, implanting, and deploying such prosthetic devices.

In one representative embodiment, a prosthetic device comprises a frame that is radially expandable and compressible between a radially compressed state and a radially expanded state. The frame comprises a plurality of posts, a plurality of links, and a plurality of compliant joints. The plurality of posts extend axially from an inflow end of the frame to an outflow end. The plurality of links extend circumferentially between adjacent posts of the plurality of posts. The plurality of compliant joints each pivotably couples one of the plurality of links to one of the plurality of posts. As the frame moves from the radially compressed state to the radially expanded state the plurality of compliant joints deflect circumferentially.

In another representative embodiment, a prosthetic device comprises a frame that is radially expandable and compressible between a radially compressed state and a radially expanded state. The frame comprises a plurality of posts and a plurality of links. Each post comprises an axially extending first member and two axially extending second members, wherein the first member is configured to move axially relative to the second members within a channel of the second members to radially expand and/or radially compress the frame. The plurality of links couple adjacent posts to one another via one or more compliant joints.

In yet another representative embodiment, an assembly comprises a prosthetic device and a delivery apparatus. The prosthetic device comprises a radially expandable and compressible frame and at least one expansion and locking device. The at least one expansion and locking device is movable between an elongated position and a retracted position and comprises a distal member and a proximal member. The proximal member comprises a locking element configured to prevent the distal member and the proximal member from moving away from one another. The distal member and the proximal member are coupled to the frame at axially spaced locations on the frame. The distal member is coupled to the frame at a more distal portion of the frame than the proximal member. The delivery apparatus comprises at least one actuation assembly configured to removably couple to the expansion and locking device and to move the distal member and the proximal member towards one another to radially expand the prosthetic device. The actuation assembly extends distally past the locking element when the expansion and locking device is in the elongated position. The distal member extends proximally past the locking element when the expansion and locking device is in the retracted position. The locking element is configured to continuously frictionally engage the actuation assembly when the actuation assembly extends distally past the locking element and is configured to continuously frictionally engage the distal member when the distal member extends proximally past the locking element to continuously lock the expansion and locking device at any position between the elongated position and the retracted position.

In yet another representative embodiment, a prosthetic device comprises a radially compressible and expandable frame comprising a plurality of posts. The plurality of posts extend axially from a proximal end of the frame to an opposite distal end of the frame, wherein each post of a subset of the plurality of posts includes a proximal member and a distal member that are axially movable relative to one another to radially expand and/or radially compress the frame. The proximal member comprises a channel that receives the distal member and within which the distal member is configured to axially slide relative to the proximal member to radially expand and/or compress the prosthetic device. The proximal member further comprises an axially extending aperture that extends distally from the proximal end of the frame to the channel, wherein the axially extending aperture is configured to receive an actuation member of a delivery apparatus. The distal member is configured to be removably coupled to the actuation member of the delivery apparatus.

In yet another representative embodiment, a prosthetic device comprises a radially compressible and expandable frame comprising a plurality of posts, a plurality of links, a plurality of first compliant joints, and a plurality of interconnects. The plurality of posts extend axially from a proximal end of the frame to an opposite distal end of the frame. The plurality of links extend circumferentially between adjacent posts of the plurality of posts. The plurality of first compliant joints each pivotably couples one of the links to one of the posts, wherein the plurality of first compliant joints comprise a plurality of first flexible neck portions. The plurality of interconnects are positioned circumferentially between the plurality of posts and comprise a plurality of second compliant joints, wherein each of the plurality of interconnects pivotably couples four of the links, and wherein the plurality of second compliant joints comprise a plurality of second flexible neck portions. One or more of the first compliant joints and/or one or more of the second compliant joints elastically deform when the frame radially expands from a radially compressed state to a partially expanded state in an initial expansion range of the frame and then plastically deforms when the frame radially expands from the partially expanded state to a fully expanded state in a subsequent expansion range.

In yet another representative embodiment, an assembly comprises a prosthetic device and a delivery apparatus. The prosthetic device comprises a radially compressible and expandable frame, the frame comprising a plurality of posts, a plurality of links, a plurality of first compliant joints, and a plurality of interconnects. The plurality of posts extend axially from a proximal end of the frame to an opposite distal end of the frame, wherein each post of a subset of the plurality of posts includes a proximal member and a distal member, wherein the proximal member comprises a channel within which the distal member is configured to axially slide relative to the proximal member to radially expand and/or compress the prosthetic device, and wherein the proximal member further comprises an axially extending aperture that extends distally from the proximal end of the frame to the channel. The plurality of links extend circumferentially between adjacent posts of the plurality of posts. The plurality of first compliant joints each pivotably couples one of the links to one of the posts. The plurality of interconnects are positioned circumferentially between the plurality of posts and comprise a plurality of second compliant joints, wherein each of the plurality of interconnects pivotable couples four of the links. The delivery apparatus comprises at least one actuation member configured to extend through the axially extending aperture of the proximal member and to removably couple to the distal member.

In yet another representative embodiment, a method, comprises radially expanding and/or compressing a prosthetic device by axially moving an actuation member of a delivery apparatus through an axially extending aperture of a post of a frame of the prosthetic device, wherein the aperture extends axially from a proximal end of the frame to a channel of a proximal member of the post within which a distal member of the post is configured to axially move, and wherein the actuation member is releasably coupled to the distal member.

In yet another representative embodiment, a prosthetic device, comprises a radially compressible and expandable frame comprising a plurality of posts, a plurality of links, a plurality of first compliant joints, and a plurality of interconnects comprising a plurality of second compliant joints. The plurality of posts extend axially from a first end of the frame to an opposite second end of the frame, wherein each post of a subset of the plurality of posts includes a first member and a second member that are axially movable relative to one another to radially expand and/or radially compress the frame. The plurality of links extend circumferentially between adjacent posts. The plurality of first compliant joints, each pivotably couples one of the links to one of the posts. The plurality of interconnects are positioned circumferentially between the plurality of posts and comprising the plurality of second compliant joints, wherein each of the plurality of interconnects is pivotably coupled to four of the links. At least one of the plurality of posts comprises a locking mechanism that is configured to prevent radial compression of the prosthetic device at prosthetic device diameters greater than a threshold prosthetic device diameter and/or wherein one or more of the first compliant joints and/or one or more of the interconnects is/are configured to help prevent radial compression of the prosthetic device at prosthetic device diameters greater than or equal to the threshold prosthetic device diameter.

In yet another representative embodiment, a prosthetic device comprises a frame that is radially expandable and compressible between a radially compressed state and a radially expanded state, the frame comprising a plurality of posts, a plurality of links, and a plurality of compliant joints. The plurality of posts extend axially from an inflow end of the frame to an outflow end. The plurality of links extend circumferentially between adjacent posts of the plurality of posts. The plurality of compliant joints each pivotably couples one of the plurality of links to one of the plurality of posts. As the frame moves from the radially compressed state to the radially expanded state the compliant joints deflect circumferentially.

In yet another representative embodiment, a prosthetic device comprises a frame that is radially expandable and compressible between a radially compressed state and a radially expanded state, the frame comprising a plurality of posts and a plurality of links. The plurality of posts each comprise an axially extending first member and two axially extending second members, the first member comprising a plurality of teeth and the second members each comprising a locking tooth configured to engage the plurality of teeth to allow movement in a first direction to allow radial expansion of the frame and prevent movement in a second direction to prevent radial compression of the frame. The plurality of links couple adjacent posts to one another via one or more compliant joints, and each compliant joint comprises a flexible neck portion configured to deflect circumferentially as the frame moves between the radially compressed state and the radially expanded state.

In yet another representative embodiment, a prosthetic device comprises a frame that is radially expandable and compressible between a radially compressed state and a radially expanded state, the frame comprising a plurality of rigid links, each link comprising a first end portion coupled to a first adjacent link via a first compliant joint and a second end portion coupled to a second adjacent link via a second compliant joint, each compliant joint comprising a flexible neck portion configured to deflect circumferentially as the frame moves from the radially compressed state to the radially expanded state.

In yet another representative embodiment, a prosthetic device comprises a frame movable between a radially compressed state and a radially expanded state. The frame comprises a first sub-frame and a second sub-frame disposed radially within the first sub-frame. Each sub-frame comprises a set of rigid links, each link comprising a first end portion coupled to a first adjacent link via a first compliant joint and a second end portion coupled to a second adjacent link via a second compliant joint. Each compliant joint comprises a flexible neck portion configured to deflect circumferentially as the frame moves from the radially compressed state to the radially expanded state. The first and second sub-frames are coupled together via a plurality of fasteners.

In yet another representative embodiment, an assembly comprises a prosthetic device and a delivery apparatus. The prosthetic device comprises a frame that is radially expandable and compressible between a radially compressed state and a radially expanded state. The frame comprises a plurality of posts and a plurality of links. One or more of the plurality of posts is/are configured as expansion and locking mechanisms and comprise an inner member including a linear rack having a plurality of teeth, and one or more outer members configured to engage the rack to allow movement in a first direction to allow radial expansion of the frame and prevent movement in a second direction to prevent radial compression of the frame. The plurality of links couple adjacent posts to one another via one or more compliant joints, and each compliant joint is configured to deflect circumferentially as the frame moves from the radially compressed state to the radially expanded state. The delivery apparatus comprises a handle, a first actuation member, and a second actuation member. The first actuation member extends from the handle and is coupled to an outflow end of the frame, the first actuation member configured to apply a distally directed force to the inner member. The second actuation member extends from the handle and is coupled to an inflow end of the frame, the second actuation member configured to apply a proximally directed force to the one or more outer members. The prosthetic device is radially expandable from the radially compressed state to the radially expanded state upon application of at least one of the distally directed force and the proximally directed force to the prosthetic device via the first and second actuation members, respectively. When the prosthetic device is in the radially expanded state the one or more outer members engage the rack to prevent compression of the frame.

In yet another representative embodiment, an assembly, comprises a prosthetic device and a delivery apparatus. The prosthetic device comprises a frame movable between a radially compressed state and a radially expanded state. The frame comprises a first sub-frame and a second sub-frame, the second sub-frame disposed radially within the first sub-frame and coupled to the first sub-frame via a plurality of fasteners. Each sub-frame comprises a set of links coupled to adjacent links via one or more compliant joints, and each compliant joint comprises a flexible neck portion configured to deflect circumferentially as the frame moves from the radially compressed state to the radially expanded state. The prosthetic device also comprises one or more expansion and locking mechanisms, each expansion and locking mechanism comprising a first member, a second member, and a locking member. The first member is coupled to the frame at a first location. The second member is coupled to the frame at a second location spaced apart from the first location and extends at least partially into the first member. The locking member is coupled to the first member and is configured to engage the second member to allow movement in a first direction to allow radial expansion of the frame and prevent movement in a second direction to prevent radial compression of the frame. The delivery apparatus comprises a handle, a first actuation member, and a second actuation member. The first actuation member extends from the handle and is coupled to the first member. The first actuation member is configured to apply a distally directed force to the first member. The second actuation member extends from the handle and is coupled to the second member. The second actuation member is configured to apply a proximally directed force to the second member. The prosthetic device is radially expandable from the radially compressed state to the radially expanded state upon application of at least one of the distally directed force and the proximally directed force to the prosthetic device via the first and second actuation members, respectively. When the prosthetic device is in the radially expanded state the locking member engages the second member to prevent compression of the frame.

In yet another representative embodiment, a method comprises inserting a distal end of a delivery apparatus into the vasculature of a patient, the delivery apparatus releasably coupled to a prosthetic device including a frame that is radially expandable and compressible between a radially compressed state and a radially expanded state, the frame comprising a plurality of posts and a plurality of links. Each post comprises an inner member and two outer members, the inner member comprising a plurality of teeth and the outer members each comprising a pawl configured to engage the plurality of teeth to allow movement in a first direction to allow radial expansion of the frame and prevent movement in a second direction to prevent radial compression of the frame. The plurality of links are configured to couple adjacent posts to one another via one or more compliant joints. The method further comprises advancing the prosthetic device to a selected implantation site, and moving at least one of the inner member distally and the outer members proximally to radially expand the prosthetic device such that the compliant joints deflect circumferentially and such that the pawls engage the plurality of teeth to lock the prosthetic device in a radially expanded state.

In yet another representative embodiment, a prosthetic device comprises a radially expandable and compressible frame, the frame comprising a plurality of posts and a plurality of links. The plurality of posts, one or more posts being configured as expansion and locking mechanisms, comprise an axially extending first member, an axially extending second member comprising a first side portion and a second side portion. The first side portion comprises a locking tooth configured to frictionally engage a surface of the first member to allow movement of the first member relative to the second member in a first direction to cause radial expansion of the frame and to prevent movement in a second direction to prevent radial compression of the frame. The plurality of links couple adjacent posts to one another via one or more compliant joints.

In yet another representative embodiment, a prosthetic device comprises a radially expandable and compressible frame, the frame comprising a plurality of posts and a plurality of links. One or more of the posts are configured as expansion and locking mechanisms and comprise an axially extending first member, an axially extending second member, and a cap. The axially extending second member comprises a first side portion and a second side portion defining a channel between them into which at least a portion of the first member extends. The cap is disposed over an outflow end portion of the second member. The cap comprises a biasing member configured to frictionally engage a surface of the first member to allow movement of the first member relative to the second member in a first direction to cause radial expansion of the frame and to prevent movement in a second direction to prevent radial compression of the frame. The plurality of links couple adjacent posts to one another via one or more compliant joints.

In yet another representative embodiment, a prosthetic device comprises a radially expandable and compressible frame, the frame comprising a plurality of posts and a plurality of links. One or more of the posts are configured as expansion and locking mechanisms and comprise an axially extending first member, an axially extending second member, and an annular cap. The axially extending second member comprises a first side portion and a second side portion defining a channel between them into which at least a portion of the first member extends. The annular cap is disposed over an outflow end portion of the second member. The cap comprises an inner lumen extending along a length of the cap, the inner lumen tapering from a first diameter at an inflow end of the cap to a second, smaller diameter at an outflow end of the cap. The plurality of links couple adjacent posts to one another via one or more compliant joints. The cap is configured to urge the first and second side portions against the first member to prevent movement of the first member relative to the second member to prevent radial compression of the frame.

In yet another representative embodiment, an assembly comprises a prosthetic device and a delivery apparatus. The prosthetic device comprises a frame that is radially expandable and compressible between a radially compressed state and a radially expanded state. The frame comprises a plurality of posts and a plurality of links. One or more of the posts are configured as expansion and locking mechanisms and comprises an axially extending first member and an axially extending second member comprising a first side portion and a second side portion. The first side portion comprises a locking tooth configured to frictionally engage a surface of the first member to allow movement of the first member relative to the second member in a first direction to cause radial expansion of the frame and to prevent movement in a second direction to prevent radial compression of the frame. The plurality of links couple adjacent posts to one another via one or more compliant joints. The delivery apparatus comprises a handle, a first actuation member, and a second actuation member. The first actuation member extends from the handle and is coupled to an outflow end of the frame. The first actuation member is configured to apply a distally directed force to the first member. The second actuation member extends from the handle and is coupled to an inflow end of the frame. The second actuation member is configured to apply a proximally directed force to the second member. The prosthetic device is radially expandable from the radially compressed state to the radially expanded state upon application of at least one of the distally directed force and the proximally directed force to the prosthetic device via the first and second actuation members, respectively. When the prosthetic device is in the radially expanded state, the locking tooth engages the first member to prevent compression of the frame.

In yet another representative embodiment, an assembly comprises a prosthetic device and a delivery apparatus. The prosthetic device comprises a frame that is radially expandable and compressible between a radially compressed state and a radially expanded state. The frame comprises a plurality of posts and a plurality of links. One or more of the posts are configured as expansion and locking mechanisms and comprise an axially extending first member, an axially extending second member, and a cap. The axially extending second member comprises a first side portion and a second side portion defining a channel between them into which at least a portion of the first member extends. The cap is disposed over an outflow end portion of the second member. The cap comprises a biasing member configured to frictionally engage a surface of the first member to allow movement of the first member relative to the second member in a first direction to cause radial expansion of the frame and to prevent movement in a second direction to prevent radial compression of the frame. The plurality of links couple adjacent posts to one another via one or more compliant joints. The delivery apparatus comprises a handle, a first actuation member, and a second actuation member. The first actuation member extends from the handle and is coupled to an outflow end of the frame. The first actuation member is configured to apply a distally directed force to the first member. The second actuation member extends from the handle and is coupled to an inflow end of the frame. The second actuation member is configured to apply a proximally directed force to the second member. The prosthetic device is radially expandable from the radially compressed state to the radially expanded state upon application of at least one of the distally directed force and the proximally directed force to the prosthetic device via the first and second actuation members, respectively. When the prosthetic device is in the radially expanded state the biasing member engages the first member to prevent compression of the frame.

In yet another representative embodiment, an assembly comprises a prosthetic device and a delivery apparatus. The prosthetic device comprises a frame that is radially expandable and compressible between a radially compressed state and a radially expanded state. The frame comprises a plurality of posts and a plurality of links. One or more of the posts are configured as expansion and locking mechanisms and comprise an axially extending first member, an axially extending second member, and an annular cap. The axially extending second member comprises a first side portion and a second side portion defining a channel between them into which at least a portion of the first member extends. The annular cap is disposed over an outflow end portion of the second member. The cap comprises an inner lumen extending along a length of the cap, the inner lumen tapering from a first diameter at an inflow end of the cap to a second, smaller diameter at an outflow end of the cap. The plurality of links couple adjacent posts to one another via one or more compliant joints. The delivery apparatus comprises a handle, a first actuation member, and a second actuation member. The first actuation member extends from the handle and is coupled to an outflow end of the frame. The first actuation member is configured to apply a distally directed force to the first member. The second actuation member extends from the handle and is coupled to an inflow end of the frame. The second actuation member is configured to apply a proximally directed force to the second member. The prosthetic device is radially expandable from the radially compressed state to the radially expanded state upon application of at least one of the distally directed force and the proximally directed force to the prosthetic device via the first and second actuation members, respectively. When the prosthetic device is in the radially expanded state the first and second side portions engage the first member to prevent compression of the frame.

In yet another representative embodiment, a method comprises inserting a distal end of a delivery apparatus into a vasculature of a patient, the delivery apparatus releasably coupled to a prosthetic device including a radially expandable and compressible frame. The frame comprises a plurality of posts and a plurality of links. One or more of the posts are configured as expansion and locking mechanisms and comprise an axially extending first member and an axially extending second member comprising a first side portion and a second side portion. The first side portion comprises a locking tooth configured to frictionally engage a surface of the first member to allow movement of the first member relative to the second member in a first direction to cause radial expansion of the frame and to prevent movement in a second direction to prevent radial compression of the frame. The plurality of links are configured to couple adjacent posts to one another via one or more compliant joints. The method further comprises advancing the prosthetic device to a selected implantation site and moving at least one of the first member distally and the second member proximally to radially expand the prosthetic device such that the compliant joints deflect circumferentially and such that the locking tooth engages the first member to lock the prosthetic device in a radially expanded state.

In yet another representative embodiment, a method comprises inserting a distal end of a delivery apparatus into a vasculature of a patient, the delivery apparatus releasably coupled to a prosthetic device including a radially expandable and compressible frame. The frame comprises a plurality of posts and a plurality of links. One or more of the posts are configured as expansion and locking mechanisms and comprise an axially extending first member, an axially extending second member comprising a first side portion and a second side portion defining a channel between them into which at least a portion of the first member extends, and a cap disposed over an outflow end portion of the second member. The cap comprises a biasing member configured to frictionally engage a surface of the first member to allow movement of the first member relative to the second member in a first direction to cause radial expansion of the frame and to prevent movement in a second direction to prevent radial compression of the frame. The plurality of links are configured to couple adjacent posts to one another via one or more compliant joints. The method further comprises advancing the prosthetic device to a selected implantation site, and moving at least one of the first member distally and the second member proximally to radially expand the prosthetic device such that the compliant joints deflect circumferentially and such that the biasing member engages the first member to lock the prosthetic device in a radially expanded state.

In yet another representative embodiment, a method comprises inserting a distal end of a delivery apparatus into a vasculature of a patient, the delivery apparatus releasably coupled to a prosthetic device including a radially expandable and compressible frame. The frame comprises a plurality of posts and a plurality of links. One or more of the posts are configured as expansion and locking mechanisms and comprise an axially extending first member, an axially extending second member comprising a first side portion and a second side portion defining a channel between them into which at least a portion of the first member extends, and an annular cap disposed over an outflow end portion of the second member. The cap comprises an inner lumen extending along a length of the cap, the inner lumen tapering from a first diameter at an inflow end of the cap to a second, smaller diameter at an outflow end of the cap. The plurality of links are configured to couple adjacent posts to one another via one or more compliant joints. The method further comprises advancing the prosthetic device to a selected implantation site, and moving at least one of the first member distally and the second member proximally to radially expand the prosthetic device such that the compliant joints deflect circumferentially and such that the first and second side portions engage the first member to lock the prosthetic device in a radially expanded state.

In yet another representative embodiment, an assembly comprises a prosthetic device and a delivery apparatus. The prosthetic device comprises a radially expandable and compressible frame and at least one expansion and locking device that is movable between an elongated position and a retracted position. The expansion and locking device comprises a distal member and a proximal member comprising a locking element configured to prevent the distal member and the proximal member from moving away from one another. The distal member and the proximal member are coupled to the frame at axially spaced locations on the frame. The distal member is coupled to the frame at a more distal portion of the frame than the proximal member. The delivery apparatus comprises at least one actuation assembly configured to removably couple to the expansion and locking device and to move the distal member and the proximal member towards one another to radially expand the prosthetic device. The actuation assembly extends distally past the locking element when the expansion and locking device is in the elongated position and the distal member extends proximally past the locking element when the expansion and locking device is in the retracted position. The locking element is configured to continuously frictionally engage the actuation assembly when the actuation assembly extends distally past the locking element and is configured to continuously frictionally engage the distal member when the distal member extends proximally past the locking element to continuously lock the expansion and locking device at any position between the elongated position and the retracted position.

In yet another representative embodiment, a prosthetic device comprises a radially expandable and compressible frame and at least one expansion and locking mechanism coupled to the frame. The expansion and locking mechanism comprises a distal member configured to be removably coupled to an actuation assembly of a delivery apparatus for the prosthetic device and a proximal member comprising a locking element configured to frictionally engage the actuation assembly and the distal member. The locking element is configured to allow movement of the actuation assembly and the distal member relative to the proximal member in a first direction to cause radial expansion of the frame and to prevent movement in a second direction to prevent radial compression of the frame. The proximal member is coupled to a more proximal portion of the frame than the distal member.

In yet another representative embodiment, an assembly comprises a prosthetic device and a delivery apparatus. The prosthetic device comprises a frame that is radially expandable and compressible between a radially compressed position and a radially expanded position, and at least one expansion and locking mechanism comprising a distal member and a proximal member. The proximal member is coupled to the frame at a more proximal portion of the frame than the distal member and comprises a locking element configured to allow movement of the distal member relative to the proximal member in a proximal direction to cause radial expansion of the frame and to prevent movement in a distal direction to prevent radial compression of the frame. The delivery apparatus comprises a handle, a first actuation member, and a second actuation member. The first actuation member is configured to apply a distally directed force to the proximal member. The first actuation member extends distally from the handle to the proximal member. The second actuation member is configured to apply a proximally directed force to the distal member. The second actuation member extends distally from the handle and is removably coupled to the distal member. The locking element frictionally engages the second actuation member when the prosthetic device is in the radially compressed position and frictionally engages the distal member when the prosthetic device is in the radially expanded position to continuously lock the prosthetic device at any position between the radially compressed position and the radially expanded position.

In yet another representative embodiment, a method comprises inserting a distal end of a delivery apparatus into a vasculature of a patient, the delivery apparatus releasably coupled to a prosthetic device comprising a radially expandable and compressible frame and an expansion and locking mechanism comprising a distal member and a proximal member. The method further comprises advancing the prosthetic device to a selected implantation site, and radially expanding the prosthetic device by: applying a distally directed force to the proximal member of the expansion and locking mechanism via a first actuation member of the delivery apparatus that is removably coupled to the proximal member, and applying a proximally directed force to the distal member of the expansion and locking mechanism via a second actuation member that extends to the proximal member. The method further comprises continuously locking the prosthetic device to prevent it from moving towards a more radially compressed position during the entire radial expansion process via a spring tooth included in the proximal member that frictionally engages the second actuation member and the distal member.

In yet another representative embodiment, a prosthetic device comprises a frame that is radially expandable and compressible between a radially compressed state and a radially expanded state. The frame comprises a plurality of links and a plurality of compliant joints. The plurality of links are connected by the plurality of compliant joints. The plurality of compliant joints each pivotably couples two of the links. As the frame moves from the radially compressed state to the radially expanded state the plurality of compliant joints deflect circumferentially.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of a prosthetic heart valve, according to one embodiment.

FIG. 2 is a side elevation view of a delivery apparatus for a prosthetic heart valve, according to one embodiment.

FIG. 3 is a perspective view of a frame of a prosthetic heart valve, according to one embodiment, with the frame shown in the partially radially expanded state.

FIG. 4 is a perspective view of the frame of FIG. 3, with the frame shown in the radially compressed state.

FIG. 5 is a perspective view of a portion of the frame of FIG. 3.

FIG. 6 is a perspective view of the frame of FIG. 3, with the frame shown in the radially expanded state.

FIG. 7A is a side elevation view of a portion of the frame of FIG. 3 showing an expansion and locking mechanism, according to one embodiment.

FIG. 7B is a side elevation view of a portion of the frame of FIG. 3 coupled to a delivery apparatus, according to one embodiment.

FIG. 8A is a side elevation view of a portion of the frame of FIG. 3.

FIG. 8B is a cross-sectional view of a commissure portion of the frame of FIG. 3 coupled to a valvular structure, according to one embodiment.

FIG. 9A is a perspective view of a frame of a prosthetic heart valve, according to another embodiment.

FIG. 9B is a cross-sectional view of a portion of the frame of FIG. 9A, including a fastener.

FIGS. 10A-10B illustrate various views of a leaf type hinge compliant joint, according to one embodiment.

FIGS. 11A-11E illustrate various views of a beam type hinge compliant joint, according to one embodiment.

FIGS. 12A-12B illustrate various views of a flat spring type hinge compliant joint, according to one embodiment.

FIG. 13 is a perspective view of a frame of a prosthetic heart valve with the frame shown in the radially expanded state, according to one embodiment.

FIG. 14 is a side elevation view of a portion of the frame of FIG. 13 showing an expansion and locking mechanism, according to one embodiment.

FIG. 15 is a side elevation view of an expansion and locking mechanism, according to another embodiment.

FIG. 16 is a side elevation view of an expansion and locking mechanism, according to still another embodiment.

FIG. 17 is a side elevation view of a portion of the expansion and locking mechanism of FIG. 16 coupled to a frame.

FIG. 18A is a schematic representation of an expansion and locking mechanism, according to another embodiment, shown in an axially elongated position.

FIG. 18B is a schematic representation of the expansion and locking mechanism of FIG. 18A shown in an axially compressed position.

FIG. 19A is a perspective view of a portion of a prosthetic heart valve frame shown in a radially compressed position that includes the expansion and locking mechanism of FIGS. 18A-18B, according to one embodiment.

FIG. 19B is a perspective view of the prosthetic heart valve frame of FIG. 19A shown in a radially expanded position.

FIG. 20 is a cross-sectional view of a locking element that may be included in the expansion and locking mechanism of FIGS. 18A-19B, according to one embodiment.

FIG. 21 is a schematic representation of an expansion and locking mechanism, according to another embodiment, shown in an axially elongated position.

FIG. 22 is a side elevation view of a portion of a prosthetic heart valve frame, according to another embodiment.

FIG. 23 is a perspective view of the prosthetic heart valve frame of FIG. 22 decoupled from an actuation member of a delivery apparatus, such as the delivery apparatus of FIG. 2.

FIG. 24 is an enlarged view of a proximal end portion of the prosthetic heart valve frame that can receive the actuation member of FIG. 23.

FIG. 25 is a perspective view of the prosthetic heart valve frame of FIGS. 22-24 coupled to the actuation member of FIGS. 23-24.

FIG. 26 is a perspective view of a joint of a prosthetic heart valve frame, according to an embodiment.

FIG. 27A is a side elevation view of the joint of FIG. 26 where the prosthetic heart valve frame is in a radially expanded and axially foreshortened state.

FIG. 27B is a side elevation view of the joint of FIGS. 26-27A where the prosthetic heart valve frame is in a radially compressed and axially elongated state.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

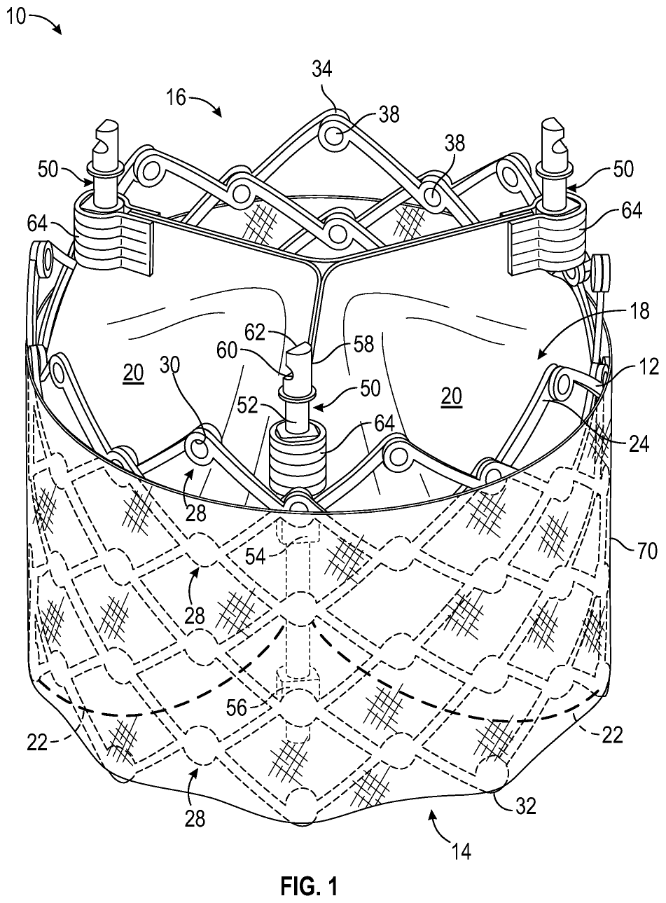

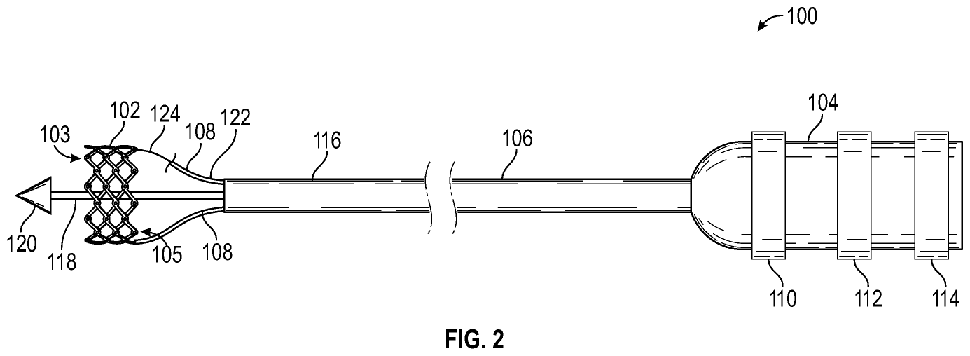

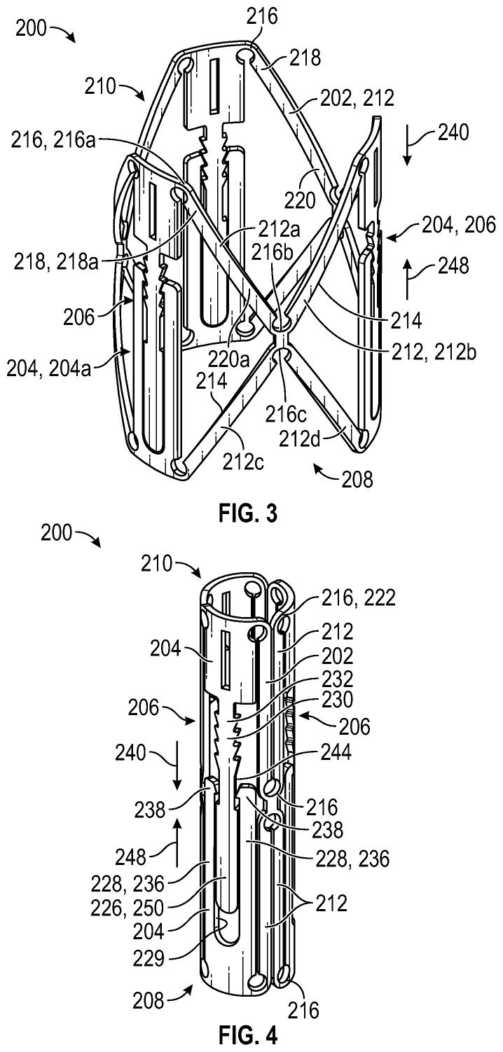

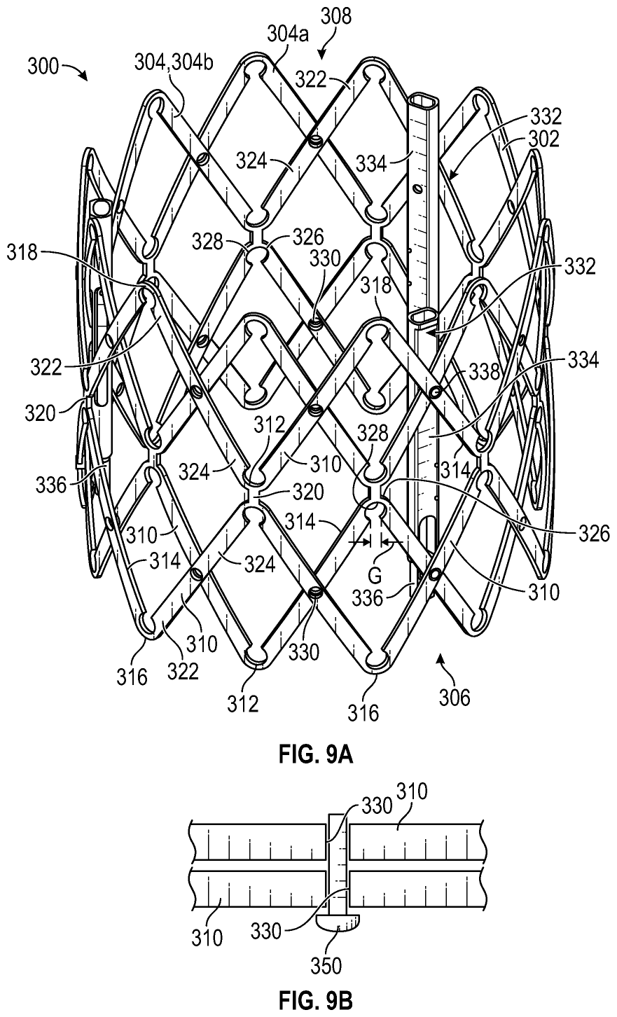

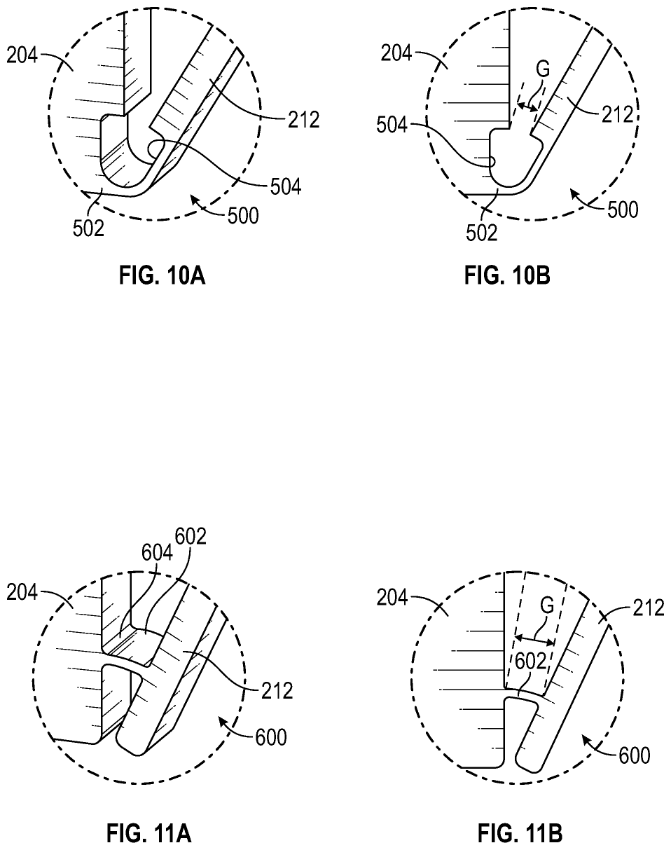

All features described herein are independent of one another and, except where structurally impossible, can be used in combination with any other feature described herein. For example, a delivery apparatus 100 as shown in FIG. 2 can be used in combination with prosthetic valves 10, 200, 300, 800, 1200, and/or 1300 described herein. In another embodiment, expansion and locking mechanisms 332 as shown in FIG. 9 can be used in combination with the prosthetic valve 200 shown in FIGS. 3-8 and/or prosthetic valve 10 shown in FIG. 1. In still other embodiments, expansion and locking mechanisms 206, 810, 900, 1000, 1100, and/or 1400 can be used with any of the disclosed prosthetic valves 10, 200, 300, 800, 1200, and/or 1300. Further, the disclosed joints and/or hinges 500, 600, 700, and 750 shown in FIGS. 10A-12B and 26-27B can be used with any one or more of the disclosed prosthetic valves 10, 200, 300, 800, 1200, and/or 1300.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

Prosthetic valves disclosed herein can be advanced through a patient's vasculature on delivery apparatuses. The prosthetic valves can include one or more expansion and locking mechanisms that can be actuated using the delivery apparatuses to radially expand the valves and lock the valves in one or more radially expanded states. As one example, the prosthetic valves can be crimped on or retained by the delivery apparatuses in a radially compressed state during delivery, and then radially expanded (and axially shortened) to a radially expanded state once the prosthetic valves reach the implantation site. It is understood that the valves disclosed herein may be used with a variety of implant delivery apparatuses, and examples thereof will be discussed in more detail later.

FIG. 1 shows an exemplary prosthetic valve that may be delivered to, and implanted at, a native heart valve by a delivery apparatus, such as the exemplary delivery apparatus shown in FIG. 2. FIGS. 3-27B show various embodiments of prosthetic valve frames and associated expansion and locking mechanisms, according the present disclosure that may be included with a prosthetic valve (such as the exemplary prosthetic valve shown in FIG. 1) to prevent the prosthetic valve from collapsing back to a more radially compressed position during and/or after radial expansion of the valve, such as during and/or after implantation of the prosthetic valve at a native heart valve. In some embodiments, the expansion and locking mechanisms of the present disclosure may be configured to continuously expand and lock the prosthetic valve at any valve diameter, allowing a physician to more smoothly expand the prosthetic valve as desired without having to worry about the valve collapsing (i.e., retracting to a more radially compressed position).

### Exemplary Embodiments of the Disclosed Technology

FIG. 1 shows an exemplary prosthetic valve 10, according to one embodiment. The prosthetic valve 10 can include an annular stent or frame 12 having an inflow end 14 and an outflow end 16. The prosthetic valve 10 can also include a valvular structure 18 which is coupled to and supported inside of the frame 12. The valvular structure 18 is configured to regulate the flow of blood through the prosthetic valve 10 from the inflow end 14 to the outflow end 16.

The valvular structure 18 can include, for example, a leaflet assembly comprising one or more leaflets 20 made of a flexible material. The leaflets 20 can be made from in whole or part, biological material, bio-compatible synthetic materials, or other such materials. Suitable biological material can include, for example, bovine pericardium (or pericardium from other sources). The leaflets 20 can be secured to one another at their adjacent sides to form commissures, each of which can be secured to a respective actuator 50 or the frame 12.

In the depicted embodiment, the valvular structure 18 comprises three leaflets 20, which can be arranged to collapse in a tricuspid arrangement. Each leaflet 20 can have an inflow edge portion 22. As shown in FIG. 1, the inflow edge portions 22 of the leaflets 20 can define an undulating, curved scallop shape that follows or tracks a plurality of interconnected strut segments of the frame 12 in a circumferential direction when the frame 12 is in the radially expanded state. The inflow edges of the leaflets can be referred to as a “scallop line.”

In some embodiments, the inflow edge portions 22 of the leaflets 20 can be sutured to adjacent struts of the frame generally along the scallop line. In other embodiments, the inflow edge portions 22 of the leaflets 20 can be sutured to an inner skirt, which in turn in sutured to adjacent struts of the frame. By forming the leaflets 20 with this scallop geometry, stresses on the leaflets 20 are reduced, which in turn improves durability of the valve 10. Moreover, by virtue of the scallop shape, folds and ripples at the belly of each leaflet 20 (the central region of each leaflet), which can cause early calcification in those areas, can be eliminated or at least minimized. The scallop geometry also reduces the amount of tissue material used to form valvular structure 18, thereby allowing a smaller, more even crimped profile at the inflow end 14 of the valve 10.

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. patent application Ser. No. 15/978,459 (Published as U.S. Publication No. 2018/0325665) and U.S. Provisional Application No. 62/854,702, filed May 30, 2019, all of which are incorporated herein by reference in their entireties.

The prosthetic valve 10 can be radially compressible and expandable between a radially compressed state (also referred to herein as the “radially compressed position” and/or the “radially compressed configuration”) and a radially expanded state (also referred to herein as the radially expanded position” and/or the “radially expanded configuration”). The frame 12 can include a plurality of interconnected lattice struts 24 arranged in a lattice-type pattern and forming a plurality of apices 34 at the outflow end 16 of the prosthetic valve 10. The struts 24 can also form similar apices 32 at the inflow end 14 of the prosthetic valve 10.

The struts 24 can be pivotably coupled to one another at one or more pivot joints or pivot junctions 28 along the length of each strut. For example, in one embodiment, each of the struts 24 can be formed with apertures 30 at opposing ends of the strut and apertures spaced along the length of the strut. Respective hinges can be formed at the locations where struts 24 overlap each other via fasteners 38, such as rivets or pins that extend through the apertures 30. The hinges can allow the struts 24 to pivot relative to one another as the frame 12 is radially expanded or compressed, such as during assembly, preparation, or implantation of the prosthetic valve 10.

The frame struts and the components used to form the pivot joints of the frame 12 (or any frames described below) can be made of any of various suitable materials, such as stainless steel, a cobalt chromium alloy, or a nickel titanium alloy (“NiTi”), for example Nitinol. In some embodiments, the frame 12 can be constructed by forming individual components (e.g., the struts and fasteners of the frame) and then mechanically assembling and connecting the individual components together. Further details regarding the construction of the frame and the prosthetic valve are described in U.S. Patent Publication Nos. 2018/0153689 and 2018/0344456, and U.S. patent application Ser. No. 16/105,353 and 62/748,284, all of which are incorporated herein by reference.

In the illustrated embodiment, the prosthetic valve 10 can be mechanically expanded from the radially contracted configuration to the radially expanded state. For example, the prosthetic valve 10 can be radially expanded by maintaining the inflow end 14 of the frame 12 at a fixed position while applying a force in the axial direction against the outflow end 16 toward the inflow end 14. Alternatively, the prosthetic valve 10 can be expanded by applying an axial force against the inflow end 14 while maintaining the outflow end 16 at a fixed position, or by applying opposing axial forces to the inflow and outflow ends 14, 16, respectively.

As shown in FIG. 1, the prosthetic valve 10 can include one or more actuators 50 mounted to and equally spaced around the inner surface of the frame 12. Each of the actuators 50 can be configured to form a releasable connection with one or more respective actuators of a delivery apparatus.

In the illustrated embodiment, expansion and compression forces can be applied to the frame by the actuators 50. Referring again to FIG. 1, each of the actuators 50 can comprise a screw or threaded rod 52, a first anchor in the form of a cylinder or sleeve 54, and a second anchor in the form of a threaded nut 56. The rod 52 extends through the sleeve 54 and the nut 56. The sleeve 54 can be secured to the frame 12, such as with a fastener 38 that forms a hinge at the junction between two struts. Each actuator 50 is configured to increase the distance between the attachment locations of a respective sleeve 54 and nut 56, which causes the frame 12 to elongate axially and compress radially, and to decrease the distance between the attachment locations of a respective sleeve 54 and nut 56, which causes the frame 12 to foreshorten axially and expand radially.

For example, each rod 52 can have external threads that engage internal threads of the nut 56 such that rotation of the rod causes corresponding axial movement of the nut 56 toward or away from the sleeve 54 (depending on the direction of rotation of the rod 52). This causes the hinges supporting the sleeve 54 and the nut 56 to move closer towards each other to radially expand the frame or to move farther away from each other to radially compress the frame, depending on the direction of rotation of the rod 52.

In other embodiments, the actuators 50 can be reciprocating type actuators configured to apply axial directed forces to the frame to produce radial expansion and compression of the frame. For example, the rod 52 of each actuator can be fixed axially relative to the nut 56 and slidable relative to the sleeve 54. Thus, in this manner, moving the rod 52 distally relative to the sleeve 54 and/or moving the sleeve 54 proximally relative to the rod 52 radially compresses the frame. Conversely, moving the rod 52 proximally relative to the sleeve 54 and/or moving the sleeve 54 distally relative to the rod 52 radially expands the frame.

When reciprocating type actuators are used, the prosthetic valve can also include one or more locking mechanisms that retain the frame in the expanded state. The locking mechanisms can be separate components that are mounted on the frame apart from the actuators, or they can be a sub-component of the actuators themselves.

Each rod 52 can include an attachment member 58 along a proximal end portion of the rod 52 configured to form a releasable connection with a corresponding actuator of a delivery apparatus. The actuator(s) of the delivery apparatus can apply forces to the rods for radially compressing or expanding the prosthetic valve 10. The attachment member 58 in the illustrated configuration comprises a notch 60 and a projection 62 that can engage a corresponding projection of an actuator of the delivery apparatus.

In the illustrated embodiments, the prosthetic valve 10 includes three such actuators 50, although a greater or fewer number of actuators could be used in other embodiments. The leaflets 20 can have commissure attachments members 64 that wrap around the sleeves 54 of the actuators 50. Further details of the actuators, locking mechanisms and delivery apparatuses for actuating the actuators can be found in U.S. Pat. Nos. 10,806,573 and 10,603,165, U.S. Patent Publication Nos. 2018/0153689 and 2018/0325665, and PCT Application Nos. PCT/US20/57691 and PCT/US21/22467 filed Nov. 28, 2020 and Mar. 16, 2021, respectively, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves disclosed herein.

The prosthetic valve 10 can include a skirt assembly comprising one or more skirts or sealing members. In some embodiments, prosthetic valve 10 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. As shown in FIG. 1, the prosthetic valve 10 can also include an outer skirt 70 mounted on the outer surface of the frame 12. The outer skirt 70 can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in PCT Application No. PCT/US20/24559 filed Mar. 25, 2020, which is incorporated herein by reference in its entirety.

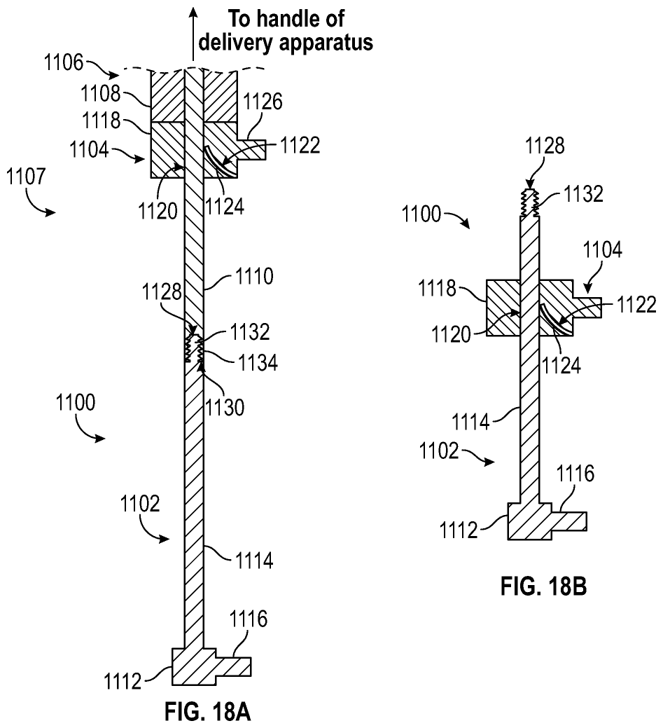

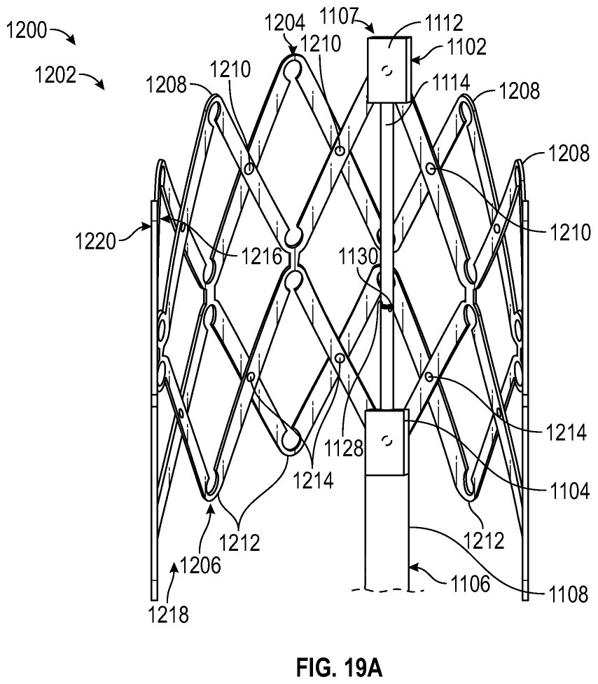

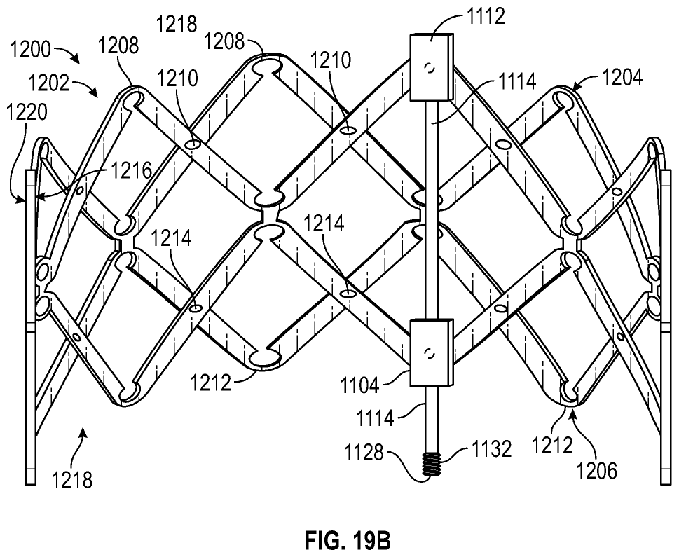

FIG. 2 illustrates a delivery apparatus 100, according to one embodiment, adapted to deliver a prosthetic heart valve 102, such as the illustrated prosthetic heart valve 10, described above. The prosthetic valve 102 can be releasably coupled to the delivery apparatus 100, such as via a removable coupling between a distal member of an expansion and locking mechanism of the prosthetic valve 102 and a second actuation member of an actuation assembly of the delivery apparatus 100, as will be described in greater detail below with reference to at least FIGS. 18A-20. The prosthetic valve 102 can include a distal end 103 and a proximal end 105, wherein the proximal end 105 is positioned closer to a handle 104 of the delivery apparatus 100 than the distal end 103, and wherein the distal end 103 is positioned farther from the handle 104 than the proximal end 105. It should be understood that the delivery apparatus 100 and other delivery apparatuses disclosed herein can be used to implant prosthetic devices other than prosthetic valves, such as stents or grafts.

The delivery apparatus 100 in the illustrated embodiment generally includes the handle 104, a first elongated shaft 106 (which comprises an outer shaft in the illustrated embodiment) extending distally from the handle 104, at least one actuator assembly 108 extending distally through the outer shaft 106, a second elongated shaft 118 (which comprises an inner shaft in the illustrated embodiment) extending distally from the handle through the first shaft 106, and a nosecone 120 connected to a distal end of the second shaft 118. The second shaft 118 and the nosecone 120 can define a guidewire lumen that is configured to receive a guidewire so that the delivery apparatus 100 can be advanced over the guidewire through the vasculature of a patient. The at least one actuator assembly 108 can be configured to radially expand and/or radially collapse the prosthetic valve 102 when actuated, and may be removably coupled to the prosthetic heart valve 102, as will be described in greater detail below.

Though the illustrated embodiment shows two actuator assemblies 108 for purposes of illustration, it should be understood that one actuator 108 can be provided for each actuator on the prosthetic valve. For example, three actuator assemblies 108 can be provided for a prosthetic valve having three actuators. In other embodiments, a greater or fewer number of actuator assemblies can be present.

In some embodiments, a distal end portion 116 of the shaft 106 can be sized to house the prosthetic valve in its radially compressed, delivery state during delivery of the prosthetic valve through the patient's vasculature. In this manner, the distal end portion 116 functions as a delivery sheath or capsule for the prosthetic valve during delivery,

Each actuator assembly 108 can comprise a support tube or sleeve 122 and an actuation member 124. The sleeve 122 can engage and/or abut the proximal end 105 of the prosthetic valve 102 while the actuation member 124 can be releasably coupled to a corresponding actuation member of the prosthetic valve. In some embodiments, the actuator assembly 108 also can include a locking tool. When actuated, the actuator assembly 108 can transmit pushing and/or pulling forces to portions of the prosthetic valve to radially expand and collapse the prosthetic valve. Specifically, the sleeve 122 can provide a distally directed pushing force to the proximal end 105 of the prosthetic valve 102 while the actuation member 124 can provide a proximally directed pulling force to an axially movable member of the prosthetic valve 102, which, in turn can transfer this proximally directed pulling force to the distal end 103 of the prosthetic valve 102. In this way, the countervailing forces provided by the sleeve and the actuation member draw the proximal and distal ends 105,103 of the prosthetic valve 102 towards one another, thereby causing the prosthetic valve 102 to radially expand. The actuator assemblies 108 can be at least partially disposed radially within, and extend axially through, one or more lumens of the outer shaft 106. For example, the actuator assemblies 108 can extend through a central lumen of the shaft 106 or through separate respective lumens formed in the shaft 106.

The handle 104 of the delivery apparatus 100 can include one or more control mechanisms (e.g., knobs or other actuating mechanisms) for controlling different components of the delivery apparatus 100 in order to expand and/or deploy the prosthetic valve 102. For example, in the illustrated embodiment the handle 104 comprises first, second, and third knobs 110, 112, and 114.

The first knob 110 can be a rotatable knob configured to produce axial movement of the outer shaft 106 relative to the prosthetic valve 102 in the distal and/or proximal directions in order to deploy the prosthetic valve from the delivery sheath 116 once the prosthetic valve has been advanced to a location at or adjacent the desired implantation location with the patient's body. For example, rotation of the first knob 110 in a first direction (e.g., clockwise) can retract the sheath 116 proximally relative to the prosthetic valve 102 and rotation of the first knob 110 in a second direction (e.g., counterclockwise) can advance the sheath 116 distally. In other embodiments, the first knob 110 can be actuated by sliding or moving the knob 110 axially, such as pulling and/or pushing the knob. In other embodiments, actuation of the first knob 110 (rotation or sliding movement of the knob 110) can produce axial movement of the actuator assemblies 108 (and therefore the prosthetic valve 102) relative to the delivery sheath 116 to advance the prosthetic valve distally from the sheath 116.

The second knob 112 can be a rotatable knob configured to produce radial expansion and/or contraction of the prosthetic valve 102. For example, rotation of the second knob 112 can move the actuation member 124 and the support tube 122 axially relative to one another. Rotation of the second knob 112 in a first direction (e.g., clockwise) can radially expand the prosthetic valve 102 and rotation of the second knob 112 in a second direction (e.g., counterclockwise) can radially collapse the prosthetic valve 102. In other embodiments, the second knob 112 can be actuated by sliding or moving the knob 112 axially, such as pulling and/or pushing the knob.

The third knob 114 can be a rotatable knob configured to retain the prosthetic heart valve 102 in its expanded configuration. For example, the third knob 114 can be operatively connected to a proximal end portion of the locking tool of each actuator assembly 108. Rotation of the third knob in a first direction (e.g., clockwise) can rotate each locking tool to advance the locking nuts to their distal positions to resist radial compression of the frame of the prosthetic valve, as described above. Rotation of the knob 114 in the opposite direction (e.g., counterclockwise) can rotate each locking tool in the opposite direction to decouple each locking tool from the prosthetic valve 102. In other embodiments, the third knob 114 can be actuated by sliding or moving the third knob 114 axially, such as pulling and/or pushing the knob. As further described below, in certain embodiments, the frame of the prosthetic valve can include a locking element or member that engages an actuator of the frame to maintain the prosthetic valve in its radially expanded state, in which case a locking tool need not be included.

Although not shown, the handle 104 can include a fourth rotatable knob operative connected to a proximal end portion of each actuation member. The fourth knob can be configured to rotate each actuation member 124, upon rotation of the knob, to unscrew each actuation member from the proximal portion of a respective actuator of the frame of the prosthetic valve. As described above, once the locking tools and the actuation members are uncoupled from the prosthetic valve 102, they can be removed from the patient.

FIGS. 3-7 illustrate an exemplary embodiment of a prosthetic heart valve 200 comprising a unitary, fastener-free frame 202. The prosthetic valve can include a valvular structure comprising a plurality of leaflet (such as valvular structure 18 comprising leaflets 20) and inner and/or outer skirts, as previously described, though these components are omitted for purposes of illustration. In the illustrated embodiment, the frame 202 includes one or more posts 204 configured as integral expansion and locking mechanisms 206 which can be used to radially expand the frame 202 and lock the frame 202 in a radially expanded state, as described in more detail below. In other embodiments, the frame 202 can comprise one or more separately formed expansion and locking mechanisms. FIG. 3 illustrates the frame 202 in a partially expanded configuration, FIG. 4 illustrates the frame 202 in a radially compressed state, and FIG. 6 illustrates the frame 202 in a fully expanded configuration.

Referring to FIG. 3, the frame 202 can comprise an inflow end portion 208 (which is the distal end of the frame in the delivery configuration for the illustrated embodiment) and an outflow end portion 210 (which is the proximal end portion of the frame in the delivery configuration for the illustrated embodiment). The frame 202 can be a mechanically expandable frame comprising a plurality of posts 204 coupled together by a plurality of rigid members or links 212 (which can also be referred to as “struts”). For example, in the illustrated embodiment, the frame 202 comprises three posts 204, and each pair of adjacent posts 204 is coupled together by four links 212 defining two generally triangularly shaped cells 214. In the illustrated embodiment, the links 212 are disposed in an X-shape. However, in other embodiments, the frame 202 can comprise a greater or fewer number of posts 204 and/or links 212, and the posts 204 and/or links 212 can be disposed in any of a variety of shapes.

Each link 212 can be coupled to one or more adjacent links 212 and/or to an adjacent post 204 via a compliant hinge or joint 216 formed integrally with the frame 202. Each link 212 can have a first end portion 218 and a second end portion 220. The first end portion 218 can be coupled to a post 204 via a first compliant joint 216 and the second end portion 220 can be coupled to an adjacent link 212 via a second compliant joint 216. For example, an exemplary first link 212a can have a first end portion 218a coupled to a first post 204a via a first compliant joint 216a, and a second end portion 220a coupled to an adjacent second link 212b via a second compliant joint 216b. The compliant joint 216b can be coupled to an additional compliant joint 216c connecting links 212c and 212d. The compliant joints 216 can be configured to allow movement of the links 212 relative to one another and/or the posts 204 via elastic body deformation of the compliant joints 216.

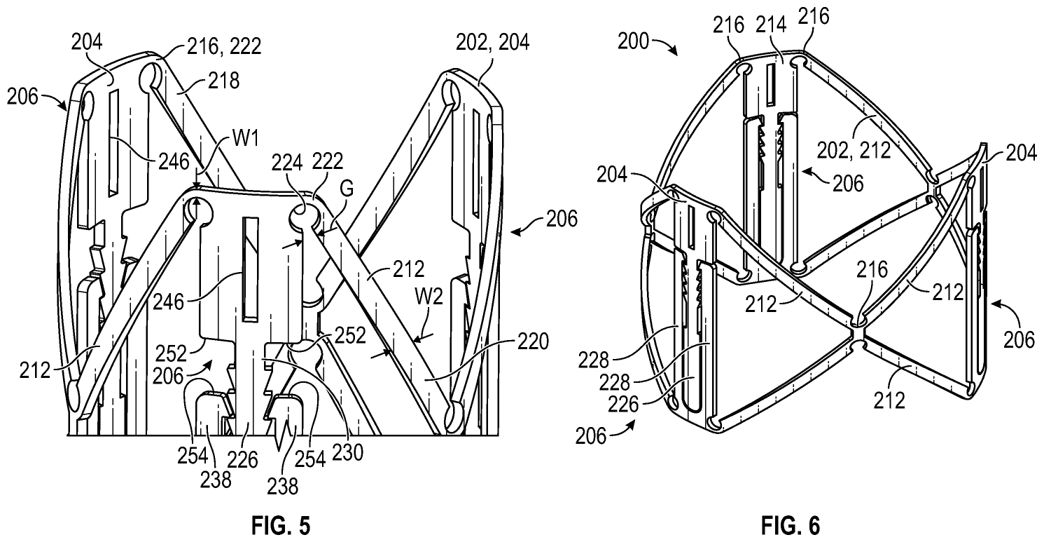

As best seen in FIG. 5, in the illustrated embodiment, each compliant joint 216 can comprise a flexible neck portion 222 at least partially defining a C-shaped cutout 224 including a gap G. In alternative embodiments, the cutouts 224 can have other shapes, such as a V-shape, an oval, square, etc., which are open at one end to define a gap G. As shown in the illustrated embodiment, the compliant joints 216 coupled to the same link portion 212 can be oriented in opposing directions (e.g., the gap G can face in different directions). For example, referring to FIG. 3, link 212a can have a first compliant joint 216a oriented in a first direction (e.g., toward the inflow end 208 of the prosthetic valve 200) and a second compliant joint 216b oriented in a second direction (e.g., toward the outflow end 210 of the prosthetic valve 200). In other embodiments, the compliant joints 216 coupled to the same link 212 can be oriented in the same direction.

As the frame 202 moves from a compressed configuration (FIG. 4) to a partially expanded configuration (FIG. 3) or fully expanded configuration (FIG. 3), the flexible neck portion 222 can deform or deflect circumferentially and the gap G can widen. Specifically, the flexible neck portion 222 can be more flexible than the links 212 and the posts 204, and thus the flexible neck portion 222 bends and/or deforms more readily responsive to an applied force or moment. As one example, the flexible neck portion 222 is more flexible due to it having a width W₁ that is narrower than a width W₂ of each link portion 212. This configuration allows the frame 202 to move between the radially compressed and radially expanded states without the use of fasteners to couple adjacent links 212 to one another. Further, the compliant joints 216 can be in-line or flush with the links 212 (e.g., the compliant joints do not protrude past the radially inner and/or radially outer surfaces of the links 212), thereby reducing the overall crimp profile of the prosthetic valve 200.

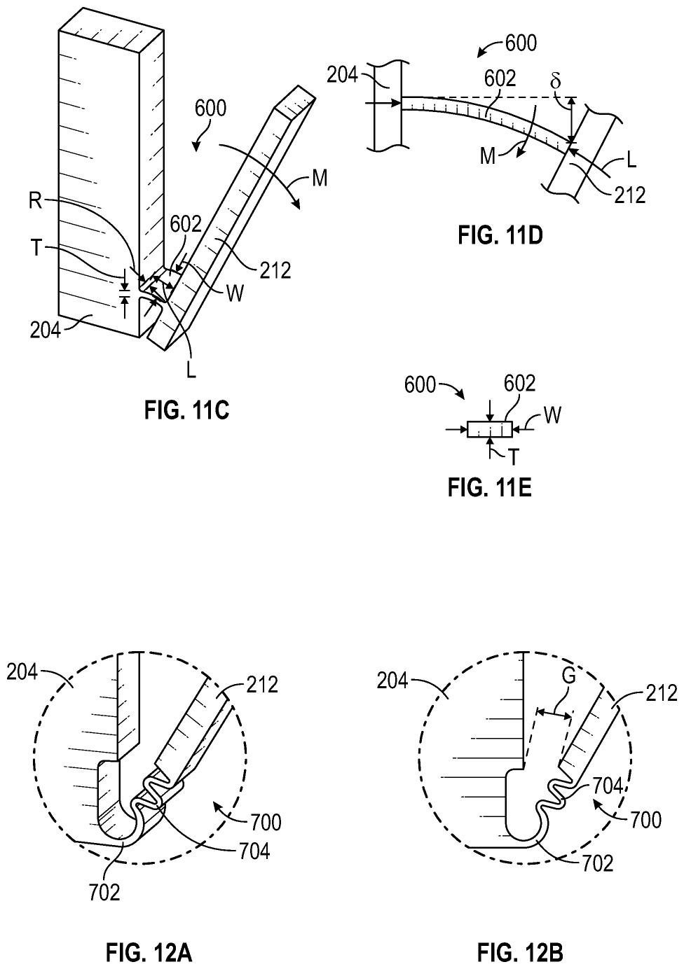

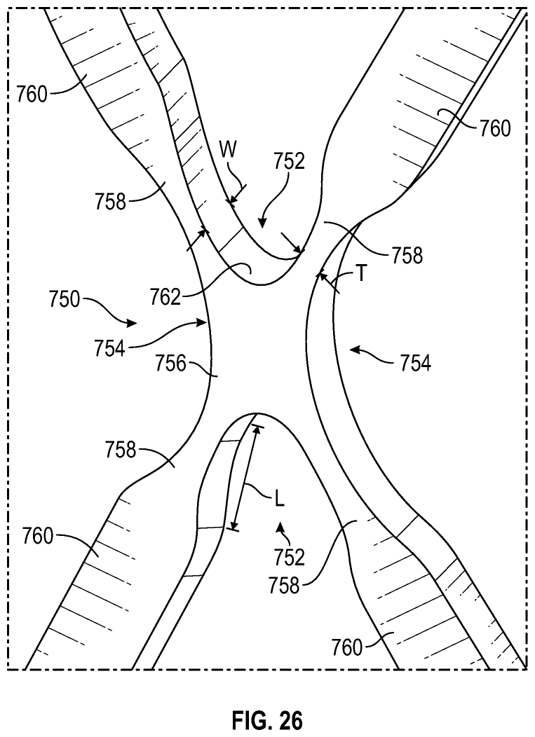

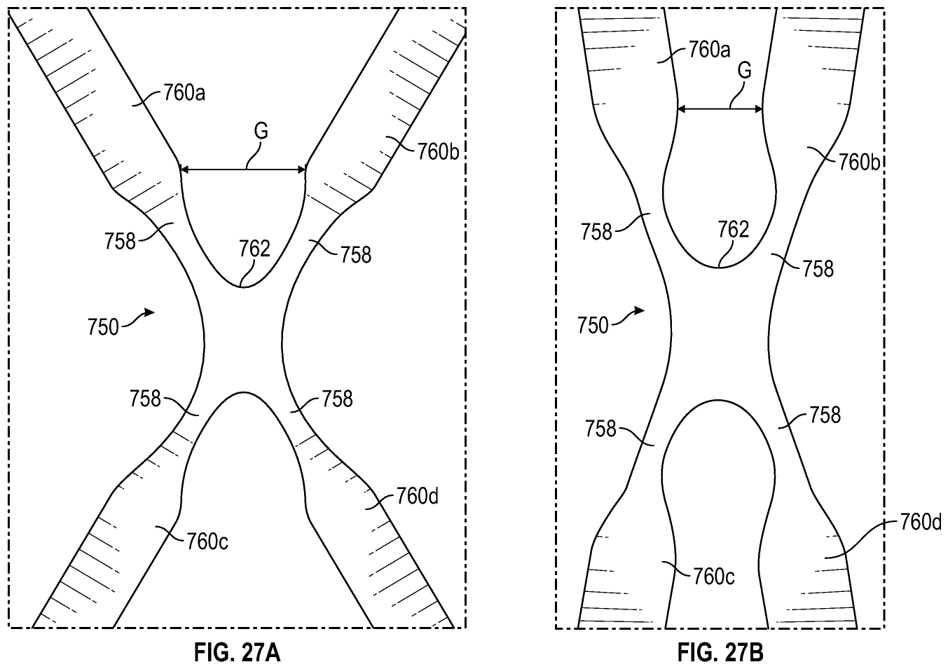

However, in other embodiments other forms of compliant joints or flexure linkages can be utilized. In some embodiments, such as the illustrated embodiment, each compliant joint 216 can comprise the same type of hinge. However, in other embodiments, a frame can comprise one or more different hinge types. Examples of different flexures or hinge types can include leaf type hinges 500 (FIGS. 10A-10B) and 752 (FIGS. 26-27B), beam-type hinges 600 (FIGS. 11A-11E), flat spring-type hinges 700 (FIGS. 12A-12B), circular flexures, elliptical flexures, corner-filleted flexures, cross-flexures (also referred to as crossed leaf-type hinges), prismatic crossed hinges, notched hinges, multi-trapezoidal hinges (also referred to as butterfly hinges), filleted V-shaped flexures, cyclonoidal flexures, circularly curved beam flexures, spherical flexures, and/or selective compliance hinges. Further details of compliant hinges that can be used with the disclosed frame embodiments can be found in, for example, International Publication No. WO 2009/034551, Linß et al., (2019), Modeling and Design of Flexure Hinge-Based Compliant Mechanisms, Kinematics—Analysis and Applications (pp. 1-24), and/or Rad, F. (2014), Design and Characterization of Curved and Spherical Flexure Hinges for Planar and Spatial Compliant Mechanisms (Research Doctorate), University of Bologna, each of which is incorporated by reference herein in its entirety.

Although some of the joints and hinges shown in FIGS. 10A-12B and 26-27B are described as being included in valve 200, it should be appreciated that these joints and/or hinges can be included in any of the other valves 10, 300, 800, 1200, and/or 1300 disclosed herein.

The geometry of the compliant joints and/or hinges and/or the materials from which the compliant joints and/or hinges are constructed can be selected to provide desired characteristics and/or properties (e.g., elasticity, plasticity, strength, etc.). For example, some or all the compliant joints and/or hinges can be formed such that they are fully elastic, partially elastic (also referred to as “partially plastic”), and/or fully plastic. Although both the materials and the geometry can contribute to the elasticity and/or plasticity of the compliant joints and/or hinges, the geometry can have a bigger influence on the elasticity and/or plasticity of the compliant joints and/or hinges than the materials, as described below with reference to FIGS. 11A-11E.

Fully elastic joints and/or hinges remain flexible and elastic under all operating conditions (i.e., all valve diameters from the fully compressed state to the fully expanded state), and thus can allow the frame 202 to radially expand and/or compress when subjected to loads, such as radial compressive loads that can be exerted on the frame 202 by surrounding tissue when implanted at a native valve. As such, when the frame 202 includes fully elastic joints and/or hinges, the frame 202 can include locking mechanisms to prevent the frame 202 from collapsing towards a radially compressed position. When the joints and/or hinges are constructed to be fully elastic, the frame 202 can be made of a super-elastic or shape memory material, such as Nitinol.

Partially elastic compliant joints and/or hinges behave elastically up to a threshold (i.e., their yield point/strength or elastic limit) during valve expansion but can lose elasticity and behave plastically (deform plastically) beyond this threshold (yield point). Within the elastic range of the frame, the prosthetic valve can be more easily re-compressed with less force during an implantation procedure as compared with the plastic range of the frame. The geometries of the joints can be selected to reach the elastic limit of the frame material at a predetermined diameter of the frame during valve expansion depending on the extent to which re-compressibility is desired. For example, if it is desired to be able to re-compress the valve even at substantially large valve diameters (e.g., so that a physician can re-compress and/or re-position the prosthetic valve even when it has almost been fully radially expanded) the geometry of the joints can be selected so that the frame 202 remains elastic under a wide range of valve diameters and only plastically deforms (i.e., becomes irreversibly plastic) when the frame 202 is at or near the fully radially expanded state. For example, the joints can be constructed from materials having higher yield points and/or the shape and/or size of the joints can be modified so that the joints more evenly distribute loads along the joints (so that stress in the joints does not concentrate in one particular area of the joints) and the joints can deform elastically throughout at least an initial expansion range of the prosthetic valve. For example, as explained below, by making the corners of the joints curved and/or less sharp, the stress along the corners can be reduced since the curved corners can more evenly distribute an applied load than sharp (orthogonal) corners.

Once in the frame is in the plastic deformation range, the joints and/or hinges are more rigid and can assist in retaining the prosthetic valve in its final radially expanded state inside the body. In this manner, when the joints and/or hinges become plastically deformable, they contribute a locking function to the prosthetic valve. In some embodiments, a frame can have partially elastic compliant joints and/or hinges without additional locking components to assist in retaining the prosthetic valve in the final expanded state and instead can rely on the plasticity of the joints and/hinges exhibiting plastic characteristics to retain the prosthetic valve in the expanded state. In other embodiments, a frame can have partially elastic compliant joints and/or hinges in addition to locking components such that the prosthetic valve can be retained in the final expanded state by a combination of the locking components and the plasticity of the joints and/or hinges.

However, it should be appreciated that the geometry of the hinges (and thus the elastic and plastic ranges of the hinges) can be selected depending on the valve diameter at which re-compressibility is not needed and/or locking is desired. A geometry that reaches the elastic limit of the material at greater valve diameters offers a wider range of valve diameters at which the valve can be reversibly compressed and/or expanded within an elastic range but offers a narrower range of diameters at which the valve can be locked, and vice versa. Partially elastic/partially plastic compliant joints and/or hinges can include portions that are fully plastic and other portions that are fully elastic. The frame 202 can include the same type of hinge at all the compliant joints 216, or the frame 202 can include a mix of different types of hinges at the compliant joints 216 depending on the particular characteristics that are desired. When the joints and/or hinges are constructed to be partially elastic, the frame 202 can be made of a plastically deformable material, such as stainless steel or a cobalt chromium alloy (e.g., a nickel-cobalt-chromium alloy such as MP35N) and the joints are configured to deform elastically within an initial expansion range and then deform plastically within a subsequent expansion range.

In other embodiments, the frame can be fully plastic, meaning that the compliant joints and/or hinges deform plastically through the entire range of valve expansion. When the joints and/or hinges are constructed to be fully plastic, the frame 202 can be made of a plastically deformable material, such as stainless steel or a cobalt chromium alloy (e.g., a nickel-cobalt-chromium alloy such as MP35N).

FIGS. 10A-10B illustrate an exemplary leaf type hinge 500. The leaf type hinge 500 can comprise a flexible neck portion 502 at least partially defining a bell-shaped cutout 504 including a gap G (FIG. 10B). As the frame 202 moves between the compressed configuration to an expanded configuration, the flexible neck portion 502 can deform or deflect circumferentially such that the gap G can widen.

FIGS. 11A-11E illustrate an exemplary beam type hinge 600 coupling a link 212 and a post 204. Specifically, FIGS. 11A and 11C are perspective views of the beam type hinge 600, FIGS. 11B and 11D are side elevation views of the beam type hinge 600, and FIG. 11E is a cross-sectional view of a flexible neck portion 602 of the beam type hinge 600 taken along a cutting plane orthogonal to the longitudinal axis of the flexible neck portion (an axis that runs along the length of the flexible neck portion 602). The beam type hinge 600 can comprise the flexible neck portion 602 extending between the link 212 and the post 204. The flexible neck portion 602 can have a length L, a width W, and a thickness T. Further, in some examples, the flexible neck portion 602 and the post 204 can form curved corners 604 where they meet.

The radius R of these curved corners can also have an impact on the durability and elasticity and/or plasticity of the hinge 600. Making the corners curved rather than orthogonal can reduce stress on the end of the flexible neck portion 602 that abuts the post 204 (the post end), thereby improving the durability of the hinge 600. Increasing the radius R of the curved corners 604 (i.e., making the corners less sharp) can also make the corners 604 more flexible and/or elastic. That is, the corners 604 will remain elastic under a greater range of valve diameters and will not start to become plastic until the valve is expanded to larger diameters. In some examples, the radius R of the corners (the radius of the circle created if the corners were extended to create full circles) can be at least half of the thickness of the flexible neck portion 602.

The flexible neck portion 602 can define a gap G (FIG. 11B) between the link 212 and the post 204 (or, alternatively, between two adjacent links 212). As the frame 202 moves from the radially compressed state towards the radially expanded state, the flexible neck portion 602 can deform or deflect such that the gap G widens. Specifically, as shown in FIGS. 11C-11D, the moment caused by an axially compressive load can pivot the link 212 relative to the post 204 about a rotation axis determined by the deflection of the flexible neck portion 602. Thus, the links 212 can pivot relative to the longitudinal axis of the frame 202 during radial expansion and/or compression of the frame 202, while the posts 204 remain aligned with the longitudinal axis of the frame 202. As such, the end of the flexible neck portion 602 that abuts the link 212 (the link end) deflects towards the nearest end of the frame (the flexible neck portions 602 included at the inflow end portion of the frame deflect towards the inflow end of the frame and the flexible neck portions 602 included at the outflow end portion of the frame 202 deflect towards the outflow end of the frame) as the frame radially expands, while the end of the flexible neck portion 602 that abuts the post 204 (the post end) can stay stationary since the post 204 remains axially aligned during radial expansion. As discussed above, the elasticity and/or plasticity of the hinge 600 can depend more on the geometry (length, width, and thickness) of the flexible neck portion 602 than the materials from which it is constructed. As an approximation, it can be assumed that the flexible neck portion 602 behaves similarly to a restrained beam under the action of an applied moment. Thus, the deflection of the end of the flexible neck portion 602 that abuts the link 212 (link end) can be approximated using the equation for the deflection of the unsupported end of a restrained beam, which is as follows:

$$Deflection of Link End of Flexible Neck Portion ( δ ) \approx \frac{6 {ML}^{2}}{{EWT}^{3}}$$

In the above equation, M is the moment, L is the length of the flexible neck portion 602, W is the width of the flexible neck portion, T is the thickness of the flexible neck portion 602, and E is the modulus of elasticity. The bending stress of the flexible neck portion 602 can similarly be approximated using the bending stress equation for a restrained beam, given by:

$$σ = \frac{M}{Z}$$

In the above equation, M is the moment and Z is the section modulus.

As such, the geometry of the flexible neck portion 602 can be selected to provide the desired elasticity and/or plasticity. As one example, the thickness of the flexible neck portion 602 can be substantially less than its width and length and/or substantially less than the thickness and/or width of the post 204 and the link 212. In some examples, the flexible neck portion 602 can be constructed from cobalt chromium alloys, nitinol, and/or other elastic and/or plastically deformable materials.

FIGS. 12A-12B illustrate an exemplary flat spring-type hinge 700. The flat spring-type hinge 700 can comprise a flexible neck portion 702 configured as a spring 704 comprising a plurality of undulating or sinusoidal segments. The spring-type hinge 700 can define a gap G (FIG. 12B) between the link 212 and the post 204 (or, alternatively, between two adjacent links 212). As the frame 202 moves between the compressed configuration to an expanded configuration, the flexible neck portion 702 can deform or deflect such that the gap G can widen.

While the above hinges 500, 600, 700 are shown connecting one of the links 212 and one of the posts 204, the hinges also can be used to connect two or more links 212 to one another. For example, as shown in FIG. 3, compliant joints 216b and 216c each connect two of the links 212. Specifically, compliant joint 216b can connect links 212a and 212b and compliant joint 216c can connect links 212c and 212d. Further, the compliant joints 216b and 216c can be connected to one another via a stem, body, or post forming a four-way joint/connection between the links 212a-d.

FIGS. 26-27B show an example of one such four-way joint. Specifically, FIG. 26 shows a perspective view of a four-way joint or interconnect 750, while FIGS. 27A-B show side elevation views of the joint 750 where the valve in which it is included is in a radially expanded position (FIG. 27A) and a radially compressed state (FIG. 27B).

Interconnect 750 can include two or more compliant joints 752, 754 joined together by a stem or hub 756. The compliant joints 752 can face opposite directions along an axis parallel to the central longitudinal axis of the frame (e.g., one can face towards the inflow end of the valve and the other can face towards the outflow end of the valve). The compliant joints 754 face in opposite directions along an axis perpendicular to the longitudinal axis of the frame. The joints 752 desirably define acute angles between adjacent links 760 and the joints 754 desirably define obtuse angles between adjacent links 760 throughout the expansion range of the frame. In other embodiments, however, the angles defined by joints 752 can be obtuse angles and the angles defined by joints 754 can be acute angles, or the angles defined by joints 752 can be the same as the angles defined by joints 754. As the frame radially expands, the angles defined by the joints 752 can increase, while the angles defined by the joints 754 can decrease, and vice versa when the frame radially compresses.

Each of the compliant joints 752, 754 can be similar to the other C-shaped compliant joints 216 and, for example, can be similar to the leaf type hinge 500. Specifically, like the leaf type hinge 500, each of the compliant joints 752, 754 can include flexible neck portions 758 that can be thinner than links 760 and that can define bell-shaped cutout 762 and gap G (FIGS. 27A-B). Each of the flexible neck portions 758 can have a width W (in the radial direction), a length L, and a thickness T (measured in a direction perpendicular to the length L.

As shown in FIGS. 27A-B, as the valve moves from a radially compressed state (FIG. 27B) towards a radially expanded state (FIG. 27A), the links 760a,b deflect away from one another and the links 760c,d deflect away from one another such that the gap G widens. Specifically, the flexible neck portions 758 are more flexible/elastic than the links 760 and/or hub 756 (e.g., because they are thinner than the links 760 and/or hub 756) and thus bend and/or deform as the valve moves between the radially compressed state and the radially expanded state, thereby widening or narrowing the gap G between the links 760. Specifically, as the valve moves from a radially compressed state (FIG. 27B) to a radially expanded state (FIG. 27A) the links 760a,b deflect away from one another and the gap G between them widens. Similarly, the links 760c,d deflect away from one another and the gap G between them widens.

Like the other joints, the joint 750 can be configured to be fully plastic, partially plastic, and/or fully elastic. In some examples, it can be particularly desirable for the joint 750 to be at least partially plastic so that it can help lock the valve in the radially expanded state (with or without additional locking components). In some examples, the flexible neck portions 758 can be wider (in a radial direction) than they are thick. In some such examples, the width W of the flexible neck portions 758 can be about 1.2 times the thickness T of the flexible neck portions 758. However, other and/or additional geometries can be selected so that the flexible neck portions 758 are elastic for a wide range of valve diameters and only become plastically deformable at a desired valve diameter (e.g., when the valve is close to and/or at the fully radially expanded state). In some examples, the yield point of the flexible neck portions 758 can be increased (so that the joint 750 remains elastic under a wider range of valve diameters) by decreasing the thickness of the flexible neck portions 758 and increasing the length and/or width of the flexible neck portions 758.

In the example shown in FIGS. 26-27B, the flexible neck portions 758 are thinner than the links 760, but just as wide as the links 760. However, in other examples, the flexible neck portions 758 can have other geometries, such as being thinner than (not as thick as) and narrower than (not as wide as) the links 760.

The joint 750 can be constructed from similar materials to the other compliant joints, such as cobalt chromium alloys, stainless steel, or other suitable plastically deformable materials. If constructed to be fully elastic, the joint 750 can be constructed from Nitinol or similar super elastic materials.

Such configurations advantageously eliminate separately formed fasteners (which can be difficult to manufacture and/or install at such a small size) and allow for a single degree of freedom (the pivotable movement between the links 212), thereby preventing or mitigating radial displacement of the links 212, and thus deformation of the frame 202. The elimination of separately formed fasteners advantageously reduces friction and wear on the frame 202, thereby increasing long-term reliability and precision of the prosthetic valve 200.

In some embodiments, the frame 202 can be formed from a unitary piece of material. For example, the frame 202 can be formed using simpler processing and machining procedures such as laser cutting, waterjet cutting, etc. In some particular embodiments, the frame 202 can be cut (e.g., laser cut) from a tube of material, which can made of any suitable biocompatible metals, such as stainless steel, a nickel-cobalt-chromium alloy (e.g., MP35N), or a nickel-titanium alloy (e.g., Nitinol). In other embodiments, the tube of material can be made of any suitable biocompatible polymeric materials. Furthermore, the absence of fasteners significantly reduces the number of components and simplifies the complexity of assembly, thereby reducing both material and time costs.

Referring to FIG. 3, as mentioned previously, in some embodiments, each post 204 can be configured as an expansion and locking mechanism 206. In the illustrated embodiment, the frame 202 comprises three posts 204, each of which is configured as an expansion and locking mechanism 206. In other embodiments, the frame 202 can comprise a greater or fewer number of posts, a selected number of which can be configured as expansion and locking mechanisms.

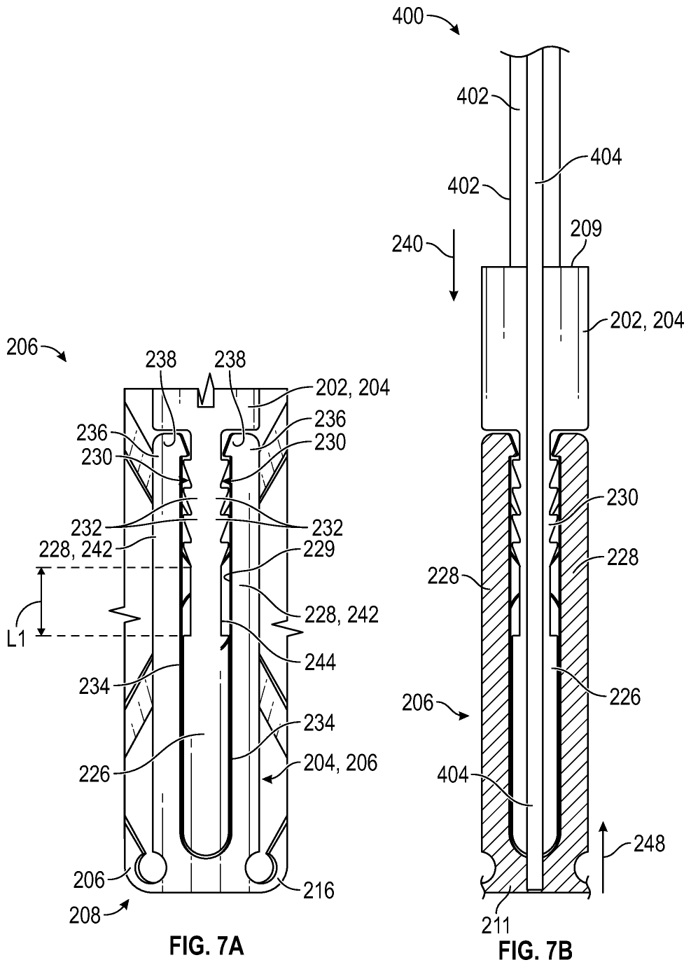

As best seen in FIGS. 7A-7B, each expansion and locking mechanism 206 can comprise a rachet mechanism or rachet assembly comprising an inner member 226 and one or more outer members 228. The outer members 228 can define a channel 229 within which the inner member 226 is configured to move (e.g., slide) axially relatively to the outer members 228. The inner and/or outer members 226, 228 can extend from and be formed integrally with a respective end of the frame 202. For example, in the illustrated embodiment, the inner member 226 extends from the outflow end 210 of the frame 202 toward the inflow end 208 and the one or more outer members 228 extend from the inflow end 208 of the frame toward the outflow end 210. In other embodiments, the inner member 226 can extend from the inflow end 208 of the frame 202 and the one or more outer members 228 can extend from the outflow end 210 of the frame. In still other embodiments, the inner and/or outer members 226, 228 can be formed separately from the frame 202 and coupled thereto using, for example, welding, adhesives, and/or mechanical fasteners such as screws or pins.

The inner member 226 can comprise one or more linear racks 230 each including a plurality of teeth 232. The inner member 226 can comprise an elongated member including first and second circumferential edges 234 (FIG. 7A). The one or more linear racks 230 can be disposed on one or more circumferential edges 234 of the inner member 226. In the illustrated embodiment, the inner member 226 comprises two linear racks 230, one disposed on each circumferential edge 234 of the inner member 226. However, in other embodiments, the inner member 226 can comprise only a single linear rack 230. In the illustrated embodiment, the linear rack 230 extends only partially along the length of the inner member 226, however, in other embodiments, the linear rack 230 can extend along the entire length of the inner member 226.

One or more of the outer members 228 can comprise a pawl 236 configured to engage the teeth 232 of the linear rack(s) 230. The pawl 236 and the teeth 232 are configured such that when the pawl 236 is engaged with the rack 230, the inner member 226 and the one or more outer members 228 can move relative to one another in a first axial direction, but are prevented from moving relative to one another in a second, opposite axial direction. For example, in the illustrated embodiment, when the pawl 236 is engaged with the linear rack 230, the inner member 226 can move axially in a distal direction (e.g., down in the orientation shown in FIG. 7) but cannot move axially in a proximal direction (e.g., up in the orientation shown in FIG. 7). This ensures that when the pawl 236 is engaged with the rack 230, the frame 202 can be radially expanded but cannot be radially compressed. In other words, the inflow end 208 and the outflow end 210 of the frame 202 can move axially toward one another but cannot move axially away from one another.

Once the prosthetic valve 200 has been implanted within a selected implantation site within a patient, the patient's native anatomy (e.g., the native aortic annulus) may exert radial forces against the prosthetic valve 200 that would tend to compress the frame 202. However, the engagement between the pawl 236 and the rack 230 prevents such forces from compressing the frame 202, thereby ensuring that the frame remains locked in the desired radially expanded state (also referred to herein as the “radially expanded position”).

In the illustrated embodiment, each expansion and locking mechanism 206 comprises two outer members 228 disposed such that they define a space between them into which at least a portion of the inner member 226 can extend. That is, the two outer members 228 can be disposed circumferentially on either side of the inner member 226, as shown in FIG. 7A. In the illustrated embodiment, each outer member 228 comprises a pawl 236 configured to engage a respective linear rack 230 of the inner member 226. In other embodiments wherein the inner member 226 comprises a single linear rack 230, only one of the outer members 228 can comprise a corresponding pawl 236. In still other such embodiments, the expansion and locking mechanism 206 can comprise only a single outer member 228, which can be disposed adjacent to the linear rack 230 such that the pawl 236 can engage the linear rack.

Each pawl 236 can comprise an elongated body terminating in a locking tooth 238 that can engage the teeth 232 of the linear rack 230. As shown, the locking tooth 238 can have a shape that is complimentary to the shape of the teeth 232, such that the tooth 232 allows sliding movement of the inner member 226 in one direction relative to the pawl 236 (e.g., downwards in the illustrated embodiment, as shown by arrow 240 in FIG. 4) and resists sliding movement of the inner member 226 in the opposite direction (e.g., upward in the illustrated embodiment) when the tooth 238 is in engagement with one of the teeth of the linear rack 230.

Each outer member 228 can be biased toward the inner member 226 such that the locking tooth 238 of each pawl 236 is resiliently retained in a position engaging one of the teeth 232 of the inner member 226 (which can be referred to as the “engaged position” of the pawl). In the illustrated embodiment, the body 242 of each outer member 228 is configured as a leaf spring. The biased configuration of the body 242 ensures that under normal operation, the locking teeth 238 remain engaged with their respective linear racks 230.

In some embodiments, such as the illustrated embodiment, the inner member 226 can comprise a toothless portion 244 adjacent the linear rack 230. The toothless portion 244 can be disposed nearer the inflow end than the linear rack 230, and can be a recessed, flat portion of the inner member, as shown. The toothless portion 244 is configured to allow bi-directional axial movement (in the distal and proximal directions) of the inner member 226 relative to the outer members 228. This allows the frame 202 to expand and/or compress prior to engagement of the pawl(s) 236 with the plurality of teeth 232. The length Li (FIG. 7A) of the toothless portion 244 can be selected to provide a reversibility range in which the prosthetic valve 200 can be freely expanded and compressed without locking.

The inner member 226 can be axially movable relative to the outer members 228 in a distal direction (e.g., toward the inflow end 208 of the frame in the orientation shown in FIG. 3) and a proximal direction (e.g., toward the outflow end 210 of the frame in the orientation shown in FIG. 3). As such, because the inner member 226 and the outer members 228 are secured to the frame 202 at axially spaced locations (the inflow end 208 and the outflow end 210, respectively), moving the inner member 226 and the outer members 228 axially with respect to one another in a telescoping manner can cause radial expansion or compression of the frame 202. For example, moving the inner member 226 distally toward the inflow end 208 of the frame while holding the outer members 228 in a fixed position and/or moving the outer members 228 proximally toward the outflow end 210 of the frame can cause the frame 202 to foreshorten axially and expand radially. Conversely, moving the inner member 226 proximally toward the outflow end of the frame and/or moving the outer members 228 distally causes the frame 202 to elongate axially and compress radially.

Each expansion and locking mechanism 206 can advantageously be configured to be in-line or flush with the frame 202 (e.g., the expansion and locking mechanisms 206 do not protrude past the radially inner and/or radially outer surfaces of the frame 202), thereby reducing the overall crimp profile of the prosthetic valve.

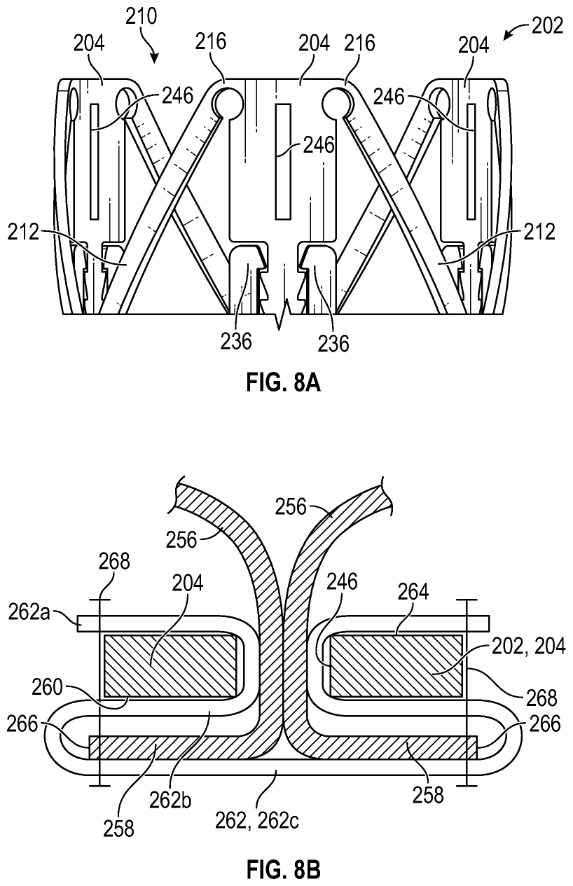

Referring to FIG. 8A, in some embodiments, each post 204 can further comprise a commissure opening or slot 246. The commissure opening 246 can extend radially through a thickness of the post 204 and can be configured to accept a portion of a valvular structure to couple the valvular structure to the frame 202. In the illustrated embodiment, the commissure opening 246 has a rectangular shape and is fully enclosed by the post 204 (e.g., the opening 246 does not extend to an inflow and/or outflow edge of the post 204). However, in other embodiments, the commissure opening 246 can have any of various shapes (e.g., square, oval, square-oval, triangular, L-shaped, T-shaped, C-shaped, etc.). In some embodiments, the opening 246 can extend to an edge of the post 204 (e.g., an outflow edge) such that a portion of the valvular structure can be slid axially (rather than radially) into the commissure opening 246.

In some embodiments, a valvular structure including a plurality of leaflets 256 can be coupled to the frame 202 in the following exemplary configuration. FIG. 8B illustrates a cross-sectional view of a portion of a post 204 of the frame 202. Tab portions 258 of adjacent leaflets 256 can extend through the commissure opening 246 and can be folded along the radially outer surface 260 of the frame 202. A flexible connector 262 (e.g., comprising a fabric) can extend along a radially inner surface 264 of the frame 202, through the opening 246, around the outer edges 266 of each tab 258, and across the radially outer surface of the tabs 258, such that the flexible connector forms a plurality of layers (e.g., first, second, and third layers 262a, 262b, 262c in the illustrated embodiment). The various components can be coupled together using one or more sutures 268. For example, in the illustrated embodiment, each suture 268 extends through the first and second layers 262a, 262b of the flexible connector, through the leaflet tab 258, and through the third layer 262c. Further details regarding commissures and additional commissure configurations useable with frame 202 can be found, at least, in U.S. Pat. No. 9,393,110 and U.S. Publication Nos. 2018/0325665 and 2019/0105153, which are incorporated by reference herein in their entirety. The inflow or cusp edges of the leaflets 256 can be connected to an inner and/or outer skirt via sutures, such as shown in FIG. 1, and/or to adjacent links 212.

The prosthetic valve 200 including fastener-free frame 202 and integral expansion and locking mechanisms 206 can be expanded in the following exemplary manner. Generally, the prosthetic valve 200 is placed in a radially compressed state and releasably coupled to a distal end portion of a delivery apparatus, such as delivery apparatus 100 (FIG. 2), and then advanced through the vasculature of a patient to a selected implantation site. For example, when replacing the native aortic valve, the prosthetic valve 200 and the distal end of the delivery apparatus can be advanced through the aorta to position the prosthetic valve 200 within the native aortic valve annulus. If retained within a delivery sheath, the delivery sheath can be retracted, or the prosthetic valve can be advanced distally from the sheath. The prosthetic valve 200 can then be expanded to its desired functional size and locked into place using the expansion and locking mechanisms 206. The prosthetic valve 200 can also be delivered and implanted within the other native valves of the heart (the mitral valve, the tricuspid valve, and the pulmonary valve) using any known delivery approaches.

Each expansion and locking mechanism 206 can be releasably coupled to a respective actuation assembly 400 of a delivery apparatus, similar to actuation assemblies 108 of delivery apparatus 100. Referring now to FIG. 7B, in a particular example, an actuation assembly 400 can comprise a first actuation member or support tube 402 (similar to support tube 122) and a second actuation member 404 (similar to actuation member 124), such as in the form of a rod, tension member, suture or pull cable. A distal end portion of the first actuation member 402 can engage or abut an outflow end portion 209 of the inner member 226 and the second actuation member 404 can be releasably coupled to an inflow end portion 211 of the outer member 228. For example, the distal end portion of the second actuation member 404 can have a threaded section that is tightened into a corresponding threaded bore of the outer member 228. The second actuation member 404 can extend through the first actuation member 402. The proximal end portions of the first and second actuation members 402, 404 can be operatively connected to a handle of the delivery apparatus. The delivery apparatus in this embodiment can include the same features described above for delivery apparatus 100.

The delivery apparatus can be used to apply a distally directed force (as shown by arrow 240) to the outflow end portion 209 of the expansion and locking mechanism 206 via the first actuation member 402 and a proximally directed force (as shown by arrow 248) to the inflow end portion 211 of the expansion and locking mechanism via the second actuation member 404 to move the inner member 226 and the outer members 228 axially relative to one another in a telescoping manner to cause the frame to radially expand.

Referring now to FIG. 4, when the frame 202 is in the radially compressed state, the inner member 226 can move relative to the outer members 228 in the proximal and/or distal directions. As the inner member 226 moves, the locking teeth 238 can slide along a distal end portion 250 of the inner member 226 and/or the toothless portion 244 until the locking teeth 238 engage the linear racks 230, as shown in FIG. 3. The engagement of the pawls 236 with the plurality of teeth 232 allows for continued radial expansion of the frame but prevents radial compression of the frame.

The frame 202 can continue to be expanded by moving the inner member 226 distally and/or the outer members 228 proximally until a selected prosthetic valve diameter is achieved. The selected diameter can correspond to a selected position of the locking teeth 238 of the pawls 236 in which they engage any tooth of the plurality of teeth 232 of the linear rack 230. For example, in the illustrated embodiment, as shown in FIG. 6, the selected diameter (e.g., a fully expanded diameter) corresponds with the position of the locking teeth 238 in which they engage the fourth tooth 232 of each linear rack 230. In other embodiments, the selected diameter (e.g., a partially expanded diameter) can correspond to the position of the locking teeth 238 where any of the other teeth 232 are engaged.

Referring to FIG. 5, in some embodiments, such as the illustrated embodiment, the inner member 226 can comprise one or more stopping surfaces 252 provided on the inner member 226. The stopping surfaces 252 can be disposed at a location proximal to the linear racks 230 and can be positioned to engage an outflow edge 254 of each pawl 236 to prevent further distal movement of the inner member 226 relative to the outer members 228 to prevent over-expansion of the prosthetic valve 200. Once the selected diameter of the prosthetic valve 200 has been reached, the delivery apparatus can be uncoupled from the prosthetic valve 200 and removed from the patient's body.

Once the prosthetic valve 200 has been implanted at a selected implantation within a patient, the patient's native anatomy (e.g., the native aortic annulus) may exert radial forces against the prosthetic valve that would tend to compress the frame 202. However, the radial forces applied by the native anatomy are orthogonal to the degree of freedom of the plurality of compliant joints 216, which advantageously allows the compliant joints 216 to spread the forces across the entire frame 202, thereby preventing the radial forces from compressing the frame 202 and ensuring that the frame remains locked in the desired radially expanded shape. After expansion of the prosthetic valve 200, the second actuation members 404 can be released from the prosthetic valve, such as by unscrewing the second actuation members 404 from their corresponding threaded sections of the outer members 228 of the frame.

As noted above, in some embodiments, the frame 202 can have compliant joints 216 that deform plastically throughout the entire expansion range or that can deform plastically only after the frame has been partially expanded. In some embodiments, as noted above, the plastically deformed joints 216 can retain the frame in the radially expanded state without locking features on the frame. For example, in such embodiments, the inner and outer members 226, 228 can be formed without any teeth that form a ratcheting mechanism therebetween. In this manner, each pair of an inner member 226 and an outer member 228 forms an expansion mechanism that is functional to expand the frame, but that does not provide a locking force for retaining the frame in the expanded state.

FIG. 9 illustrates an exemplary embodiment of a prosthetic heart valve 300 having a frame 302. The prosthetic valve 300 can further include a valvular structure (such as valvular structure 18) and inner and/or outer skirts, as previously described, though these components are omitted for purposes of illustration.

The frame 302 can be configured as two or more sub-frames 304 coupled together. Each sub-frame 304 can comprise a plurality of links 310 coupled together via compliant joints 312. Such a configuration can be advantageous in situations where a higher density of links 310 is required. For example, in situations where it is advantageous for the prosthetic valve to include smaller open cell-areas around the periphery of the prosthetic valve, for example, to bias or trap the native leaflets against the arterial wall during expansion of the prosthetic valve.

In the illustrated embodiment, the frame 302 comprises a first sub-frame 304a and a second sub-frame 304b. The frame 302 can comprise an inflow end portion 306 (which is the distal end of the frame in the delivery configuration for the illustrated embodiment) and an outflow end portion 308 (which is the proximal end portion of the frame in the delivery configuration for the illustrated embodiment). The first and second sub-frames 304 can be coupled together such that one frame is positioned radially outwardly of the other frame. For example, in the illustrated embodiment, the first sub-frame 304a is positioned radially outwardly of the second sub-frame 304b. In other embodiments, the second sub frame 304b can be positioned radially outwardly of the first sub-frame 304a. In still other embodiments, the first and second sub-frames 304 can be coupled together in a woven or latticed manner such that portions of the second sub-frame 304b are positioned radially outwardly of the first sub-frame 304a and vice versa.

Each sub-frame 304 can be similar to frame 202, described above, except that sub-frames 304 do not include posts or integral expansion and locking mechanisms. As mentioned previously, each sub-frame 304 can comprise a plurality of links 310 coupled together via compliant joints 312. In the illustrated embodiment, each sub-frame 304 comprises a plurality of diamond-shaped cells 314 each defined by four links 310. Each cell 314 can have an inflow apex 316, an outflow apex 318, and two side apices 320. Each cell 314 can be coupled to one or more adjacent cells 314 at the side apices 320. In other embodiments, each sub-frame 304 can comprise links 310 disposed in any of a variety of shapes.

Each link 310 can be coupled to one or more adjacent links 310 via a compliant hinge or joint 312 formed integrally with the frame 302. Each link 310 can have a first end portion 322 and a second end portion 324. The first end portion 322 of each link 310 can be coupled to an adjacent link 310 via a compliant joint 312 at the inflow or outflow apex 316, 318 of each cell 314, and the second end portion 324 of each link 310 can be coupled to an adjacent link 310 via a compliant joint 312 at a side apex 320. Two compliant joints 312 can be coupled together at each side apex 320. The compliant joints 312 can be configured to allow movement of the links 310 relative to one another via elastic body deformation of the compliant joints 312. In the illustrated embodiment, each compliant joint 312 can comprise a flexible neck portion 326 at least partially defining a C-shaped cutout 328 including a gap G. However, in other embodiments, each other forms of compliant joints or flexure linkages can be used, for example, any of the hinge types shown in FIGS. 10A-12B. As shown, the compliant joints 312 coupled to the same link portion 310 can be oriented in opposing directions. In other embodiments, the compliant joints 312 coupled to the same link 310 can be oriented in the same direction.

As the frame 302 moves from a compressed configuration to a fully expanded configuration, the flexible neck portion 326 can deform or deflect and the gap G can widen. The flexible neck portion 326 can have a width narrower than a width of each link portion 310. This configuration allows the frame 302 to move between the radially compressed and radially expanded states without the use of fasteners to couple adjacent links 310 to one another. Further, the compliant joints 312 can be in-line or flush with the links 310 (in contrast to fasteners which protrude from the surface of a strut), thereby reducing the overall crimp profile of the prosthetic valve 300.

In some embodiments, each sub-frame 304 can be formed from a unitary piece of material. For example, each sub-frame 304 can be formed using simpler processing and machining procedures such as laser cutting, waterjet cutting, etc. In some particular embodiments, the sub-frames 304 can each be cut (e.g., laser cut) from a tube of material, as previously described for frame 202. Furthermore, the absence of fasteners in each sub-frame significantly reduces the number of components and simplifies the complexity of assembly, thereby reducing both material and time costs.

The first and second sub-frames 304 can be coupled to one another using, for example, a plurality of fasteners such as rivets or pins. Each sub-frame 304 can comprise a plurality of openings 330 extending through links 310 in the sub-frame 304 at junctions where the first and second sub-frames 304a, 304b overlap one another. As shown in FIG. 9B, a respective fastener 350 can extend through a pair of openings 330 of a pair of overlapping links 310 to form a hinge joint between the links. Further details regarding the hinge joints are disclosed in U.S. Publication Nos. 2018/0153689 and 2018/0344456, which are incorporated herein by reference. Such a configuration may be advantageous if a higher density of links 310 is required for the frame 302, resulting in smaller open cell areas around the periphery of the valve, for example, to facilitate biasing the native leaflets against the native arterial wall during expansion of the prosthetic valve.

In some embodiments, such as the illustrated embodiment, in lieu of or in addition to integrally formed expansion and locking mechanisms (such as expansion and locking mechanisms 206 described above) the frame 302 can comprise one or more separately formed expansion and locking members 332. Though the illustrated embodiment shows three expansion and locking mechanisms 332 spaced apart from each other about the circumference of the frame, it should be noted that a prosthetic valve can comprise any number of expansion and locking mechanisms 332. For example, in some embodiments, a prosthetic valve can comprise a single expansion and locking mechanism, or two expansion and locking mechanisms, or four expansion and locking mechanisms, etc. The expansion and locking mechanisms 332 can be placed at any position about the circumference of the frame 302. For example, in some embodiments such as the illustrated embodiment, the expansion and locking mechanisms 332 are equally spaced from one another about the circumference of the frame 302. In other embodiments, it can be advantageous to have two or more expansion and locking mechanisms situated adjacent to one another.

Each expansion and locking mechanism 332 can include an outer member or sleeve 334 having an inner bore and an inner member 336 extending at least partially into the sleeve 334. A distal end portion of the inner member 336 can be coupled to the frame 302 at a first location via a fastener that is affixed to and extends radially from the distal end portion of the inner member 336. The fastener can be for example, a rivet or pin. As shown, in some embodiments, the fastener can extend through corresponding openings 330 at a junction of two overlapping links 310 of the first and second sub-frames 304a, 304b and can serve as a pivot pin around which the links 310 can pivot relative to each other and the inner member 336. The sleeve 334 can be coupled to the frame 302 at a second location, axially spaced from the first location. For example, in the illustrated embodiment, the inner member 336 is secured to the frame 302 near the distal or inflow end 306 of the frame and the sleeve 334 is secured to the frame 302 closer to or at the proximal or outflow end 308 of the frame, such as via a fastener 338 (e.g., a rivet or pin). The fastener 338 is affixed to and extends radially from the sleeve 334 through a junction of two overlapping links 310 of the first and second sub-frames 304a, 304b and can serve as a pivot pin around which the links 310 can pivot relative to each other and the sleeve 334.

The expansion and locking mechanisms 332 can further comprising a locking member configured to lock the sleeve 334 and inner member 336 such that the sleeve 334 and inner member 336 are restrained from movement relative to one another in one or more directions. Further details of expansion and locking mechanisms can be found, for example, in PCT Application No. PCT/US2020/057691, filed Oct. 28, 2020, which is incorporated herein by reference in its entirety.

The inner member 336 can be axially movable relative to the sleeve 334 in a proximal direction and in a distal direction. As such, because the inner member 336 and the sleeve 334 are secured to the frame 302 at axially spaced locations, moving the inner member 336 and the sleeve 334 axially with respect to one another in a telescoping manner can cause radial expansion or compression of the frame 302. For example, moving the inner member 336 proximally toward the outflow end 308 of the frame while holding the sleeve 334 in a fixed position and/or moving the sleeve 334 distally toward the inflow end 306 of the frame can cause the frame 302 to foreshorten axially and expand radially. Conversely, moving the inner member 336 distally toward the inflow end of the frame and/or moving the sleeve 334 proximally causes the frame 302 to elongate axially and compress radially.

As noted above, in some embodiments, the frame 202 can have compliant joints 312 that deform plastically throughout the entire expansion range or that deform plastically only after the frame has been partially expanded. In some embodiments, as noted above, the plastically deformed joints 312 can help retain the frame in the radially expanded state and/or can fully retain the frame in the radially expanded state without any other locking features on the frame. In some such embodiments, the expansion and locking mechanisms 332 can omit a locking mechanism and thus can be used only to radially expand the frame (without providing any locking functionality).

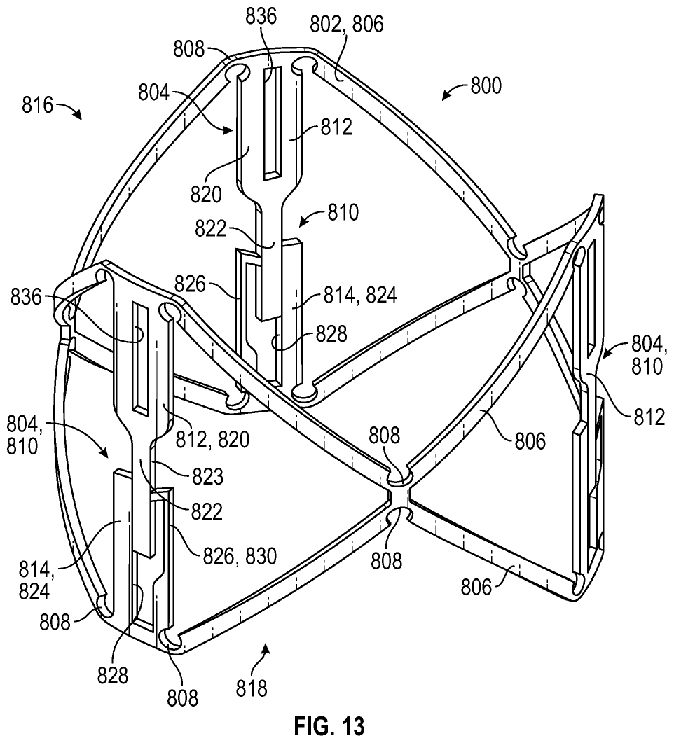

FIG. 13 illustrates an exemplary embodiment of a prosthetic heart valve 800 having a frame 802. The prosthetic valve 800 can further include a valvular structure (such as valvular structure 18) and inner and/or outer skirts, as previously described, though these components are omitted for purposes of illustration. The prosthetic valve 800 can be similar to prosthetic valve 200, described above, including a plurality of posts 804 coupled together by a plurality of rigid members or links 806 via compliant joints 808, except that in lieu of expansion and locking mechanisms 206, prosthetic valve 800 includes integral expansion and locking mechanisms 810. Expansion and locking mechanisms 810 can be used to radially expand the frame 802 and to lock the frame in a radially expanded state. The frame 802 in this embodiment can include generally the same features described above for frame 202.

The expansion and locking mechanisms 810 can be formed integrally with the frame 802, such that the frame 802 can be formed form a unitary piece of material (e.g., a tube). For example, the frame 802, including expansion and locking mechanisms 810, can be formed using simpler processing and machining procedures such as laser cutting, waterjet cutting, etc. In some particular embodiments, the frame 802 can be cut from a cobalt chromium alloy (e.g., MP35N) tube. Furthermore, the absence of separately formed expansion and locking mechanisms significantly reduces the number of components and simplifies the complexity of assembly, thereby reducing both material and time costs.

In the illustrated embodiment, the frame 802 comprises three posts 804, each of which is configured as an expansion and locking mechanism 810. In other embodiments, the frame 802 can comprise a greater or fewer number of posts 804, a selected number of which can be configured as expansion and locking mechanisms 810. Each expansion and locking mechanism 810 can comprise an inner member 812 and an outer member 814. Each inner and/or outer member 812, 814 can extend from and be formed integrally with a respective end of the frame 802. For example, in the illustrated embodiment, the inner member 812 extends from the outflow end 816 of the frame 802 toward the inflow end 818 and the outer member 814 extends from the inflow end 818 of the frame toward the outflow end 816. In other embodiments, the inner member 812 can extend from the inflow end 818 of the frame 802 and the outer member 814 can extend from the outflow end 816 of the frame.

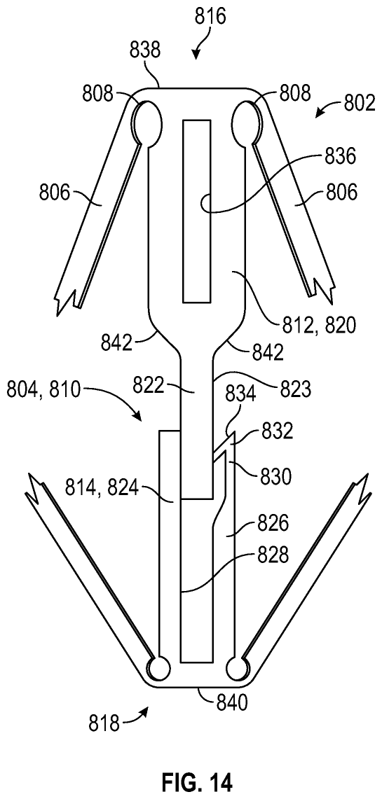

Referring to FIG. 14, the inner member 812 can comprise a main body 820 and an elongated member or rod 822. The outer member 814 can comprise a first side portion 824 and a second side portion 826 defining an opening or channel 828 between them into which at least a portion of the elongated rod 822 can extend. The second side portion 826 can be configured as a biasing member 830 (e.g., a leaf spring) ending in a V-shaped locking tooth 832. The biasing member 830 can be biased toward the rod 822, such that a first leg 834 of the V-shaped tooth 832 frictionally engages the elongated rod 822. The biased configuration ensures that under normal operation the locking tooth 832 remains engaged with the elongated rod 822.

The locking tooth 832 can be configured to frictionally engage a surface of the elongated rod 822 such that when the locking tooth 832 and the rod 822 are engaged, the inner member 812 and the outer member 814 can move relative to one another in a first axial direction, but are prevented from moving relative to one another in a second, opposite axial direction. For example, in the illustrated embodiment, when the locking tooth 832 is engaged with the rod 822 the inner member 812 can move axially in a distal direction (e.g., down in the orientation shown in FIG. 14) but is prevented from moving axially in a proximal direction (e.g., up in the orientation shown in FIG. 14). This ensures that when the locking tooth 832 is engaged with the rod 822, the frame 802 can be radially expanded but cannot be radially compressed. In other words, the inflow end 816 and the outflow end 818 of the frame 802 can move axially toward one another but cannot move axially away from one another.

Unlike the ratchet mechanisms described above, the outer surface of the rod 822 can be formed without locking features, such as teeth, that come in contact with the locking tooth 832. In the illustrated embodiment, the rod 822 has a rectangular cross-sectional profile in a plane perpendicular to the length of the rod and has a flat side surface 823 that contacts the locking tooth 832. In other embodiments, the rod 822 can have a cylindrical outer surface and can have a round cross-sectional profile in a plane perpendicular to the length of the rod

As shown in FIG. 14, in the illustrated embodiment, the locking tooth 832 can be oriented in the distal direction (e.g., with the point of the V facing the outflow end of the frame). This orientation allows the leg 834 to deflect toward the side portion 826 as the rod 822 is moved distally between the first and second side portions 824, 826 of the outer member 814. However, due to the bias of the leg 834 against the rod 822 and its orientation in the distal direction, the leg 834 can resist travel of the rod 822 in the proximal direction relative to the first and second side portions 824, 826 of the outer member 814. In other embodiments (e.g., embodiments wherein the inner member 812 extends from the inflow end portion 818 of the frame 802), the locking tooth 832 can be oriented in the proximal direction to allow proximal movement of the rod 822 but prevent distal movement. An advantage of the locking mechanism 810 of the present embodiment is that the locking tooth 832 can allow for continuous expansion and continuous locking of the frame at any diameter when the inner member is in engagement with the locking tooth. In other words, during expansion, the locking tooth can retain the frame in any expanded diameter, unlike a ratchet mechanism that is limited to locking the valve frame at discrete steps corresponding to the locking positions of the pawl relative to the teeth of the ratchet mechanism. In this manner, the locking mechanism 810 can provide a continuous expansion and locking range.

Once the prosthetic valve 800 has been implanted within a selected implantation site within a patient, the patient's native anatomy (e.g., the native aortic annulus) may exert radial forces against the prosthetic valve 800 that would tend to compress the frame 802. However, the engagement between the locking tooth 832 of the biasing member 830 and the elongated rod 822 prevents such forces from compressing the frame 802, thereby ensuring that the frame remains locked in the desired radially expanded state.

In the illustrated embodiment, only the second side portion 826 of the outer member 814 comprises a biasing member 830 and locking tooth 832, however, in other embodiments, both the first side portion 824 and the second side portion 826 can comprise respective biasing members and locking teeth.

The inner member 812 can be axially movable relative to the outer member 814 in a distal direction (e.g., toward the inflow end 818 of the frame in the orientation shown in FIG. 14). As such, because the inner member 812 and the outer member 814 are secured to the frame 802 at axially spaced locations (the inflow end 818 and the outflow end 816, respectively), moving the inner member 812 and the outer member 814 axially with respect to one another in a telescoping manner can cause radial expansion or compression of the frame 802. For example, moving the inner member 812 distally toward the inflow end 818 of the frame while holding the outer member 814 in a fixed position and/or moving the outer member 814 proximally toward the outflow end 816 of the frame can cause the frame 802 to foreshorten axially and expand radially.

In some embodiments, when the frame 802 is in the compressed configuration, the distal end of the inner member 812 can be spaced apart from the outer member 814 by a selected distance, allowing movement of the inner member 812 relative to the outer member 814 in both the distal and proximal directions. This allows the frame 802 to expand and/or compress prior to engagement of the inner member 812 with the outer member 814, at which point the frame 802 can expand further but can no longer be compressed. The length of the space between the inner member 812 and the outer member 814 can be selected to provide a reversibility range in which the prosthetic valve 800 can be freely expanded and compressed without locking.

As shown in FIGS. 13-14, in the illustrated embodiment, each post 804 further comprises a commissure opening 836. The commissure opening 836 can extend radially through a thickness of the post 804 and can be configured to accept a portion of a valvular structure to couple the valvular structure to the frame 802. In the illustrated embodiment, the commissure opening 836 has a rectangular shape and is fully enclosed by the post 804 (e.g., the opening 836 does not extend to an inflow and/or outflow edge of the post 804). However, in other embodiments, the commissure opening 836 can have any of various shapes (e.g., square, oval, square-oval, triangular, L-shaped, T-shaped, C-shaped, etc.). In some embodiments, the opening 836 can extend to an edge of the post 804 (e.g., an outflow edge) such that a portion of the valvular structure can be slid axially (rather than radially) into the commissure opening 836. In some embodiments, a valvular structure including a plurality of leaflets can be coupled to the frame in the configuration shown in FIG. 8B and described previously with respect to prosthetic valve 200.

In the illustrated embodiment, the commissure opening 836 is disposed within the inner member 812 of the post 804. However, in other embodiments (e.g., embodiments wherein the inner member extends from the inflow end 818 of the frame and the outer member 814 extends from the outflow end 816) the commissure opening 836 can be configured as part of the outer member 814.

The prosthetic heart valve 800 can be coupled to a delivery apparatus, such as delivery apparatus 100, via the expansion and locking mechanisms 810 similar to the manner shown in FIG. 7B and described above with respect to expansion and locking mechanisms 206. That is, the prosthetic valve 800 can be coupled to a delivery apparatus via a first actuation member or support tube 402 (or support tube 122) configured to engage or abut the outflow end portion 838 of the inner member 812, and a second actuation member 404 (or actuation member 124) (such as in the form of a rod, tension member or pull cable) configured to be releasably coupled to the outer member 814, as previously described. The proximal end portions of the first and second actuation members 402, 404 can be operatively connected to a handle of the delivery apparatus. The delivery apparatus in this embodiment can include the same features described above for delivery apparatus 100.

The delivery apparatus can be used to apply a distally directed force to the outflow end portion 838 of the expansion and locking mechanism 810 via the first actuation member 402 and a proximally directed force to the inflow end portion 840 of the expansion and locking mechanism via the second actuation member 404 to move the inner member 812 and the outer member 814 axially relative to one another in a telescoping manner to cause the frame to radially expand.

The prosthetic heart valve 800 can be deployed at a selected implantation site using the same method described above for prosthetic heart valve 200. The frame 802 can be expanded by moving the inner member 812 distally and/or the outer member 814 proximally until a selected prosthetic valve diameter is achieved.

In some embodiments, such as the illustrated embodiment, the inner member 812 can comprise one or more sloped or angled surfaces 842 extending from the elongated rod 822 to the main body 820. As the prosthetic valve 800 expands and the inner member 812 moves distally with respect to the outer member 814, the locking tooth 832 can slidingly advance along the angled surface 842 until the outflow end portion of the outer member 814 contacts the surfaces 842, preventing further advancement of the inner member 812 and thereby preventing over-expansion of the prosthetic valve 800. Side portions 824, 826 of the outer member 814 can be sufficiently rigid as to resist deflection away from one another. Once the selected diameter of the prosthetic valve 800 has been reached, the delivery apparatus can be uncoupled from the prosthetic valve 800 and removed from the patient's body.

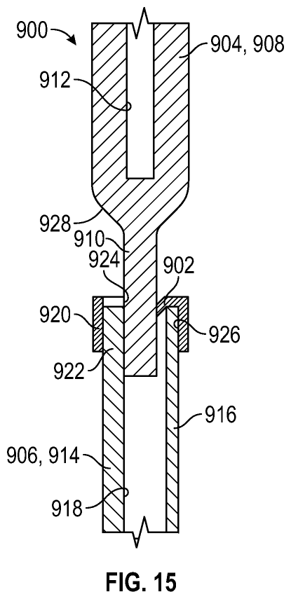

Referring to FIG. 15, in some embodiments, in lieu of or in addition to expansion and locking mechanism 810, prosthetic valve 800 can comprise expansion and locking mechanism 900. Expansion and locking mechanisms 900 can be similar to expansion and locking mechanisms 810, except that each expansion and locking mechanism 900 includes a biasing member 902 that is formed separately from the frame 802.

The expansion and locking mechanism 900 can comprise an inner member 904 and an outer member 906. Inner member 904 can be similar to inner member 812 and can comprise a main body 908 and an elongated rod 910. In some embodiments, the main body 908 can comprise a commissure opening 912 similar to commissure opening 836, described previously. The outer member 906 can comprise first and second side portions 914, 916 defining an opening or channel 918 between them, into which at least a portion of the elongated rod 910 can extend.

The expansion and locking mechanism 900 can further comprise a locking member or cap 920. The cap 920 can be a separately formed component coupled to the outflow end portion 922 of the outer member 906 and can define an opening 924 through which the elongated rod 910 can extend into the channel 918. Such a configuration can advantageously prevent or mitigate the risk of disconnection between the outer and inner members 904, 906.

As shown in FIG. 15, the cap 920 can comprise a biasing member 902 coupled to an inner surface 926 of the cap 920. The biasing member 902 can be biased toward the elongated rod 910 such that under normal operation the biasing member 902 remains engaged with the elongated rod 910. The end portion of the biasing member 902 can be configured to frictionally engage a surface of the elongated rod such that when the biasing member 902 and the rod 910 are engaged, the inner member 904 and the outer member 906 can move relative to one another in a first axial direction, but are prevented from moving relative to one another in a second, opposite axial direction. For example, in the illustrated embodiment, when the biasing member 902 is engaged with the rod 910 the inner member 904 can move axially in a distal direction (e.g., down in the orientation shown in FIG. 15) but is prevented from moving axially in a proximal direction (e.g., up in the orientation shown in FIG. 15). This ensures that when the biasing member 902 is engaged with the rod 910, the frame 802 can be radially expanded but cannot be radially compressed. In other words, the inflow end 818 and the outflow end 816 of the frame 802 can move axially toward one another but cannot move axially away from one another.

In some embodiments, such as the illustrated embodiment, the inner member 904 can comprise one or more sloped or angled surfaces 928 extending from the elongated rod 910 to the main body 908. As the prosthetic valve 800 expands and the inner member 904 moves distally with respect to the outer member 906, the cap 920 can slidingly advance along the angled surface 928 until the width of the inner member 904 is too great to advance through the opening 924, thereby preventing further advancement of the inner member 904 and preventing over-expansion of the prosthetic valve 800.

A prosthetic heart valve including expansion and locking mechanism 900 can be coupled to a delivery apparatus and expanded in the same manner as described for prosthetic heart valve 800 including expansion and locking mechanisms 810. The frame 802 can be expanded by moving the inner member 904 distally and/or the outer member 906 proximally until a selected prosthetic valve diameter is achieved.

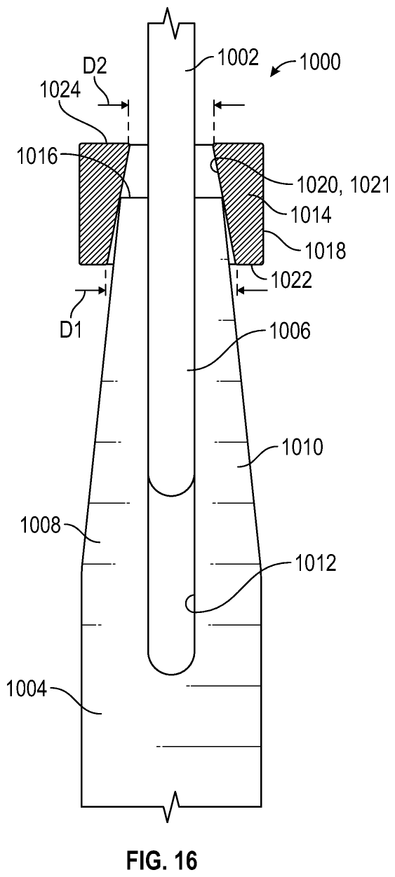

Referring to FIG. 16, in another embodiment, in lieu of or in addition to expansion and locking mechanisms 810 and/or 900, a prosthetic valve (e.g., prosthetic valve 800) can comprise expansion and locking mechanisms 1000.

Expansion and locking mechanisms 1000 can comprise an inner member 1002 and an outer member 1004. Inner member 1002 can be similar to inner member 812 and can comprise a main body (not shown) and an elongated rod 1006. In some embodiments, the inner member 1002 can comprise a commissure opening similar to commissure opening 836.

The outer member 1004 can comprise first and second side portions 1008, 1010 defining an opening or channel 1012 between them, into which at least a portion of the elongated rod 1006 can extend. The first and second side portions 1008, 1010 can be flexible such that they can resiliently bend toward or away from one another.

The expansion and locking mechanism 1000 can further comprise a locking member or cap 1014 that can be disposed over the outflow end portion 1016 of the second member 1004. The cap 1014 can be an annular member having an outer surface 1018 and an inner surface 1020 defining a lumen 1021. As shown in FIG. 16, the inner surface 1020 of the cap 1014 can be angled such that an inner diameter D₁ of the cap 1014 at the inflow end 1022 is greater than an inner diameter D₂ of the cap 1014 at the outflow end 1024. In other words, the lumen 1021 of the cap 1014 can taper from the inflow end 1022 of the cap to the outflow end 1024.

During assembly of a prosthetic valve including expansion and locking mechanisms 1000, the elongated rod 1006 can be disposed such that it extends through the lumen 1021 of the cap 1014 and into the channel 1012 between the first and second side portions 1008, 1010. The cap 1014 can be advanced over the outflow end portions 1016 of the side portions 1008, 1010 such that the inclined inner surface 1020 urges the first and second side portions 1008, 1010 toward the elongated rod 1006 such that the side portions 1008, 1010 engage the surface of the rod 1006.

In use (e.g., when expanding and/or compressing the frame 802), the inner member 1002 can move relative to the outer member 1004 within the channel 1012. When the cap is disposed on the outer member 1004, the angled inner surface 1020 of the cap 1014 prevents the inner member 1002 from moving relative to the outer member 1004.

In some embodiments, during expansion of the frame 802, the cap 1014 can be spaced apart from the outer member 1004 and the inner member 1002 can move relative to the outer member 1004 in the distal direction (e.g., down in the orientation shown in FIG. 16) and the proximal direction (e.g., up in the orientation shown in FIG. 16). Once the fame 802 has reached a selected diameter, the cap 1014 can be slid distally over the end outer member 1004 such that the side portions 1008, 1010 engage the rod 1006 and the frame 802 is prevented from further radial expansion and/or compression. The cap 1014 can be actuated (e.g., slid distally) using, for example, an actuation member of the delivery apparatus.

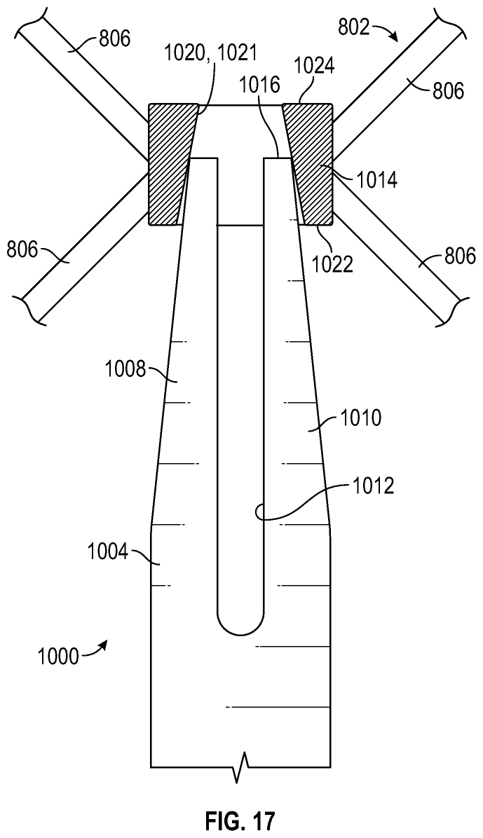

Referring to FIG. 17, in other embodiments, the cap 1014 can be coupled to a cell of the frame 802 or to the junction between adjacent cells such that the cap 1014 can move distally and/or proximally as the frame 802 is expanded and/or compressed. During expansion of the frame 802, the cap 1014 can move relative to the outer member 1004 such that the clamping force of the cap 1014 on the first and second side portions 1008, 1010 gradually increases due to the tapered inner lumen 1021 until a selected diameter is reached, at which point the inner member 1002 and outer member 1004 are restrained from movement relative to one another.

In some embodiments, the cap 1014 can be formed as a separate component from the frame 802 (which includes, e.g., the inner and outer members 1002, 1004 of the expansion and locking mechanism 1000). In other embodiments, the cap 1014 can be integrally formed with other components of the frame, such as a cell of the frame as shown in FIG. 17. For example, the cap 1014 can be formed integrally with one or more links 806 of the frame 802 such that as the frame 802 is radially expanded and/or compressed, the movement of the links 806 causes corresponding movement of the cap 1014. In the illustrated embodiment, the cap 1014 can be connected to adjacent ends of two links of a first cell and adjacent cells of two links of a second cell in the same row as the first cell such that the cap 1014 defines or is located at the junction between the first and second cells.

Advantageously, the above described expansion and locking mechanisms 810, 900, and 1000 can be formed using simpler processing and machining procedures such as laser cutting, waterjet cutting, etc. In some particular embodiments, the frame 802 (including the expansion and locking mechanisms 810, 900, 1000, and/or selected components thereof) can be cut (e.g., laser cut) from a metal tube made of, for example, a cobalt chromium alloy (e.g., MP35N), stainless steel, or Nitinol. The absence of separately formed expansion and locking mechanisms significantly reduces the number of components and simplifies the complexity of assembly, thereby reducing both material and time costs.

Furthermore, the described expansion and locking mechanisms 810, 900, 1000 can advantageously be all or partially in-line or flush with the frame 802 (e.g., the expansion and locking mechanisms do not protrude past the radially inner and/or radially outer surfaces of the frame, or only partially protrude therefrom), thereby reducing the overall crimp profile of the prosthetic valve. Still further, expansion and locking mechanisms 810, 900, and 1000 allow for continuous prosthetic valve expansion without the stepped expansion that results from a ratcheting mechanism.

FIGS. 18A-21 illustrate an expansion and locking mechanism 1100 (which also may be referred to herein as “expansion and locking device 1100,” and/or “valve deployment device”), according to yet another embodiment. Specifically, FIGS. 18A, 19A, and 21 illustrate the expansion and locking mechanism 1100 in an axially extended position, with FIG. 19A showing how the expansion and locking mechanism 1100 may hold a prosthetic valve 1200 (which also may be referred to herein as “prosthetic heart valve 1200”) in a radially compressed (and axially elongated) position when the expansion and locking mechanism is included with the prosthetic valve 1200 and adjusted to the axially extended position. FIGS. 18B and 19B illustrate the expansion and locking mechanism 1100 in an axially compressed or retracted position, with FIG. 19B showing how the expansion and locking mechanism 1100 may hold the prosthetic valve 1200 in a radially expanded (and axially foreshortened) position when the expansion and locking mechanism is included with the prosthetic valve 1200 and adjusted to the axially compressed position.

The expansion and locking mechanism 1100 is similar to expansion and locking mechanisms 810, 900, and 1000 in that it is self-locking (i.e., it allows for continuous expansion and locking of a prosthetic valve 1200 (FIGS. 19A-19B) between the radially compressed (and axially elongated) position (FIG. 19A) and the radially compressed (and axially foreshortened) position (FIG. 19B)). However, the expansion and locking mechanism 1100 is configured slightly differently than the expansion and locking mechanisms 810, 900, and 1000. For example, unlike the locking tooth 832, which is shown included in the distal member (outer member 814) of the expansion and locking mechanism 810 in FIGS. 13-14, the locking element of the expansion and locking mechanism 1100 can be included in the proximal member of the expansion and locking mechanism 1100. Further, unlike the distal member (outer member 814) of the expansion and locking mechanism 810, which is configured as the outer member of the expansion and locking mechanism 810, the distal member of the expansion and locking mechanism 1100 can be configured as the inner member (i.e., the distal member extends into and through the proximal member). Thus, in operation, the distal member of the expansion and locking mechanism 1100 can be pulled towards and through the proximal member of the expansion and locking mechanism 1100, and the locking element in the proximal member of the expansion and locking mechanism 1100 continuously holds the distal member in place and prevents the distal member from retracting (e.g., sliding back) towards a more distal position. As introduced above, the valve 1200 may be crimped onto a distal end of a delivery apparatus in the radially compressed position and delivered to the implantation site (e.g., native heart valve) in this low-profile configuration. Once at the implantation site, the valve 1200 may be expanded to the radially expanded position, which may be the operational/functional configuration of the valve 1200.

The expansion and locking mechanism 1100 comprises a distal member 1102 and a proximal member 1104 that are axially movable relative to one another. Specifically, the distal member 1102 is configured to move proximally towards, into, and/or through the proximal member 1104 to radially expand the valve 1200. Similar to the expansion and locking mechanisms 810, 900, and 1000, a physician may pull the distal member 1102 towards and/or through the proximal member 1104 via an actuation assembly 1106. Together, the actuation assembly 1106 and the expansion and locking mechanism 1100 may comprise an expansion and locking assembly 1107 (which also may be referred to herein as “valve deployment assembly 1107”). The actuation assembly 1106 may comprise a first actuation member 1108 (which also may referred to herein as “sleeve 1108,” “support tube 1108,” and/or “outer sheath 1108”) and a second actuation member 1110 (which also may be referred to herein as “tension member 1110”) slidably received within the first actuation member 1108. In particular embodiments, the second actuation member 1110 can comprise a pull cable. In other embodiments, the second actuation member 1110 can comprise a rod or wire.

The first and second actuation members 1108, 1110 may be included in the delivery apparatus (e.g., delivery apparatus 100) and may be coupled to, and/or may extend distally from, the handle (e.g., handle 104) of the delivery apparatus. The first actuation member 1108 may be configured to carry a distally directed force applied to the handle. For example, the first actuation member 1108 may be fixedly attached to the handle such that the first actuation member 1108 does not move relative to the handle. Thus, the first actuation member 1108 may move in lockstep with the handle and may transfer any distally directed force applied to the handle to a distal end of the first actuation member 1108. The second actuation member 1110 is configured to move relative the first actuation member 1108. Specifically, the second actuation member 1110 may move proximally relative to the first actuation member 1108 upon application of a proximally directed force. For example, the physician can apply a proximally directed for to the second actuation member 1110 by pulling directly on the second actuation member 1110 (in examples where the second actuation member 1110 extends proximally out of and/or past the handle) or by adjusting a control mechanism (e.g., knob 112) included on the handle of the delivery apparatus.

The second actuation member 1110 is removably coupled to the distal member 1102 of the expansion and locking mechanism 1100 to pull the distal member 1102 towards and/or through the proximal member 1104 (and thereby radially expand the valve 1200) upon application of the proximally directed force, while the first actuation member 1108 abuts the proximal member 1104 and/or a proximal portion of the prosthetic valve 1200 to facilitate valve expansion and/or hold the prosthetic valve 1200 in place as the valve 1200 is expanded upon application of the distally directed force. Specifically, while applying the proximally directed force (e.g., pulling force) to the second actuation member 1110, the physician may provide a countervailing distally directed force (e.g., pushing force) to the first actuation member 1108 to facilitate valve expansion and hold the valve 1200 in place relative to the surrounding tissue. As just one example, the physician can provide the distally directed force by gripping, holding, and/or pushing the handle (e.g., handle 104) of the delivery apparatus (e.g., delivery apparatus 100). Meanwhile, the physician can adjust (e.g., rotate, pull, slide, etc.) the control mechanism (e.g., knob 112) included in the handle, or directly pull the second actuation member 1110, to provide the proximally directed force to the second actuation member 1110. In this way, the physician can axially foreshorten and radially expand the prosthetic valve 1200 while maintaining the relative position of the prosthetic valve 1200 within the surrounding tissue.

The distal member 1102 of the expansion and locking mechanism 1100 is coupled to a prosthetic valve frame 1202 of the prosthetic valve 1200 (FIGS. 19A-19B) at a more distal position on the frame 1202 than the proximal member 1104 (i.e., a position nearer a distal end 1204 of the frame 1202 and farther from a proximal end 1206 of the frame 1202 than the proximal member 1104). For example, as shown in the embodiment illustrated in FIGS. 19A-19B, the distal member 1102 may be coupled to a distal apex 1208 of the frame 1202 located at and/or near the distal end 1204 of the frame 1202. As another example, the distal member 1102 may be coupled to a distal junction 1210 of the frame 1202. Correspondingly, the proximal member 1104 may be coupled to a proximal apex 1212 or proximal junction 1214 of the frame 1202. In this way, the distal and proximal members 1102, 1104 may be axially spaced apart from one another, at least when the frame 1202 is in a radially compressed (e.g., crimped) position, such as the radially compressed position shown in FIG. 19A. In this way, the distal member 1102 and proximal member 1104 may be drawn towards one another (as described above) to axially foreshorten and radially expand the frame 1202.

In some embodiments, the distal member 1102 and/or the proximal member 1104 may be coupled to the frame 1202 such as via fasteners (e.g., screws, pins), adhesives, sutures, thermal bonding (e.g., welding) or other suitable coupling means. In other embodiments, the distal member 1102 and/or the proximal member 1104 may be integrally formed and/or unitary with the frame 1202. In some embodiments, such as the embodiment shown in FIGS. 19A-19B, the distal member 1102 and the proximal member 1104 may be coupled to, formed with, and/or otherwise included on the inside (i.e., on an interior side 1216) of the frame 1202 within a lumen 1218 of the frame 1202.

In some such embodiments, the distal member 1102 and/or the proximal member 1104 can be configured to receive and/or retain leaflets of a valvular structure of the frame 1202. As one such example, the proximal member 1104 and/or the distal member 1102 can include a commissure opening (e.g., commissure opening 836) that receives the leaflets, and a commissure clamp or connector (e.g., flexible connector 262) may be used to couple, hold, and/or otherwise secure the leaflets to the frame 1202 to prevent them from pulling out of the commissure opening. Thus, in such embodiments, the expansion and locking mechanism 1100 also may be configured to hold the commissures of adjacent leaflets in addition to expanding and locking the prosthetic valve 1200. For example, the expansion and locking mechanism 1100 may be included as part of a commissure clamping structure (e.g., commissure post) of the valve 1200. In some such examples, the valve 1200 may include a plurality of commissure clamping structures (e.g., three commissure posts) and the expansion and locking mechanism 1100 may be incorporated and/or included in at least one of the commissure clamping structures.

However, in other such embodiments, the expansion and locking mechanism 1100 may be separate from the commissure clamping structures (e.g., commissure posts) that are configured to hold the commissures of the leaflets. In such examples, the expansion and locking mechanism 1100 need not be aligned with one or more of the commissure clamping structures along a common longitudinal axis. That is, the expansion and locking mechanism 1100 may be circumferentially spaced away from the commissure clamping structures in some such embodiments.

In other embodiments, the expansion and locking mechanism 1100 may be coupled to, formed with, and/or otherwise included on an outside (i.e., on an exterior side 1220) of the frame 1202, opposite the interior side 1216, such as between the frame 1202 and an outer skirt (e.g., outer skirt 70) of the valve 1200. In yet further embodiments, the expansion and locking mechanism 1100 may cross over the frame 1202 (between the interior and exterior sides of the frame 1202) such that it is included on both sides of the frame 1202.

The distal member 1102 may include an end portion 1112 and an elongated member or rod 1114. In some embodiments, the elongated member 1114 can comprise a wire or cable. The end portion 1112 is coupled to and/or integrally formed with the frame 1202 (e.g., at the distal apex 1208 or distal junction 1210), and the rod 1114 extends axially towards the proximal member 1104 of the expansion and locking mechanism 1100 and/or the proximal end 1206 of the frame 1202. In some such examples, the end portion 1112 may include an attachment member 1116 that is configured to be coupled to the frame 1202. For example, the attachment member 1116 may extend through an opening in the frame 1202 to help secure the distal member 1102 to the frame 1202. In some such examples, a mating mechanical fastener (e.g., nut) may be coupled to the end of the attachment member 1116 on the opposite side of the frame 1202 to prevent the attachment member 1116 from pulling out of the opening in the frame 1202. Alternatively, the attachment member 1116 may be welded to the frame 1202.

The proximal member 1104 may include a main body 1118 having an opening 1120 (which also may be referred to herein as “channel 1118” and/or “passageway 1118”) that extends through the main body 1118 and is configured to receive the rod 1114 of the distal member 1102 and/or the second actuation member 1110 of the actuation assembly 1106. That is the rod 1114 and/or the second actuation member 1110 may be configured to extend through the opening 1120 of the proximal member 1104. Specifically, and as introduced above, the rod 1114 and/or the second actuation member 1110 may be configured to be moved proximally (e.g., pulled) through the opening 1120 of the proximal member 1104 towards the handle of the delivery apparatus. The proximal member 1104 also includes a locking element 1122, which, in the embodiments illustrated in FIGS. 18A-20, comprises a spring tooth 1124 (which can have the configuration of a Wago® wire connector) that extends into the opening 1120. The locking element 1122 is configured to only permit unidirectional movement of the rod 1114 and/or distal member 1102 in the proximal direction (first axial direction) through the proximal member 1104 to ensure that the valve 1200 does not collapse back towards a more radially compressed position. That is, the locking element 1122 is configured to provide a continuous compressive and/or locking force to the rod 1114 and/or the second actuation member 1110 to prevent the distal member 1102 from moving distally (in a second axial direction) towards the elongated position, thereby holding the frame 1202 in position at any valve diameter, such as is described above with reference to FIG. 14.

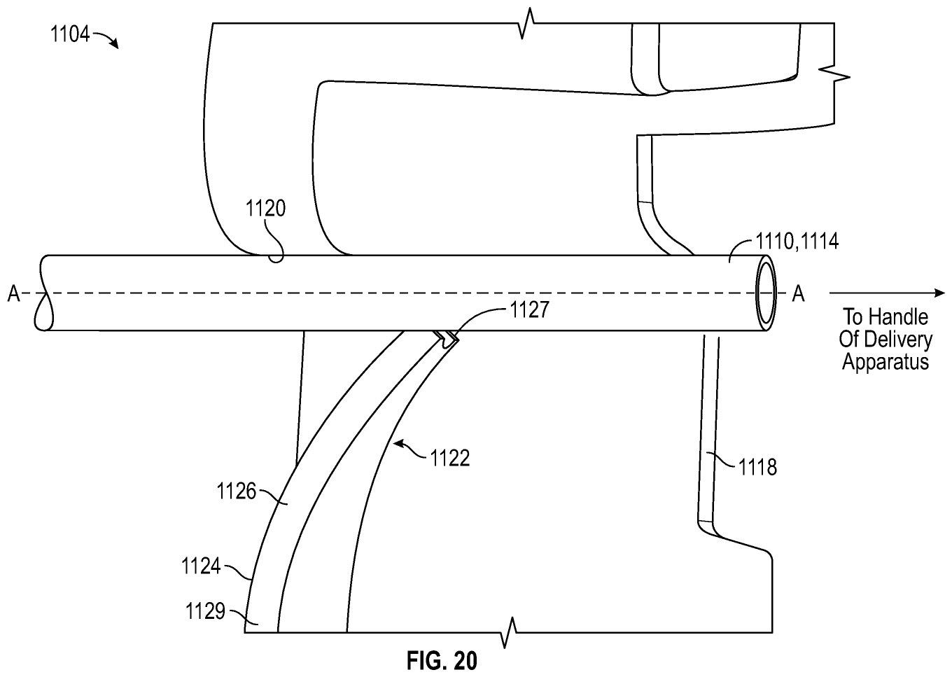

Specifically, and as best seen in FIG. 20, the spring tooth 1124 may be biased towards an extended position (shown in FIG. 20) in which the spring tooth 1124 extends into the opening 1120 of the proximal member 1104 and frictionally engages with, compresses, physically contacts, and/or otherwise exerts a holding force (e.g., pressure) on the rod 1114 and/or second actuation member 1110 to prevent relative movement between the proximal member 1104 and the distal member 1102 (and therefore prevent radial compression of the valve 1200). For example, and as explained above with reference to FIG. 14, the spring tooth 1124 may comprise a biasing member 1126 (e.g., leaf spring) (FIG. 20) that is configured to exert a constant biasing force against the rod to only permit unidirectional movement of the rod 1114 and/or second actuation member 1110 relative to the proximal member 1104 in the first axial direction. In this way, the biasing member 1126 may ensure that the spring tooth 1124 remains engaged with the rod 1114 and/or second actuation member 1110 to provide the continuous locking force, while still allowing rod 1114 and/or second actuation member 1110 to move in the first axial direction relative to the proximal member 1104 so that the valve 1200 can be radially expanded. Stated more simply, the spring tooth 1124 allows the valve 1200 to be radially expanded, but not radially compressed (i.e., it prevents the valve 1200 from collapsing back to a more radially compressed position).

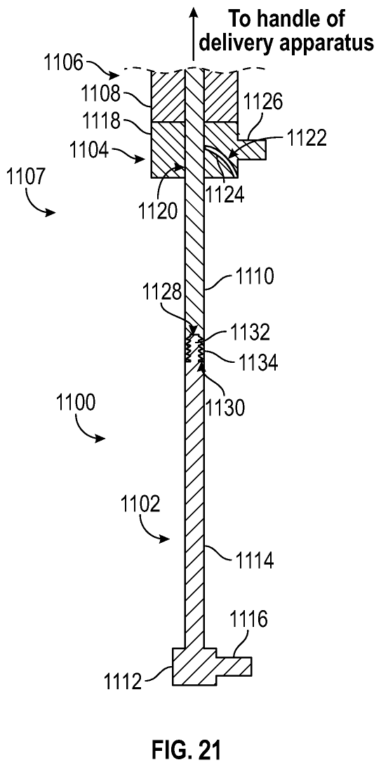

The spring tooth 1124 may only allow the rod 1114 and/or second actuation member 1110 to move in the proximal direction (first axial direction) towards the handle of the delivery apparatus (and not the second axial direction) by virtue of its shape and/or geometry. That is, even though the spring tooth 1124 exerts the holding force on the rod 1114 and/or second actuation member 1110 and prevents the rod 1114 and/or second actuation member 1110 from moving in the distal direction (second axial direction) towards the extended position, it may still allow the rod 1114 and/or second actuation member 1110 to move in the first axial direction (so that the prosthetic valve 1200 can expand) due to its shape and/or geometry. For example, the spring tooth 1124 may be curved and/or angled relative to a central longitudinal axis A-A of the opening 1120 (FIG. 20). As one such example, the spring tooth 1124 may be curved and/or angled in the first axial direction towards the handle of the delivery apparatus. In some embodiments (as shown in FIG. 20), the spring tooth 1124 may have a concave curvature with respect to the first axial direction, meaning that the concave surface of the spring tooth faces in the first axial direction. In other embodiments (as shown in FIG. 21), the spring tooth 1124 may have a convex curvature with respect to the first axial direction, meaning that the convex surface of the spring tooth 1124 faces in the first axial direction. Because the spring tooth 1124 extends in the first axial direction from a fixed end 1129 to a free end 1127 (which engages the rod 1114), the spring tooth 1124 can prevent the second actuation member 1110 and/or rod 1114 from moving in the second axial direction (distal direction) away from the handle of the delivery apparatus, but can permit the rod 1114 to slide in the first axial direction against the free end 1127 of the spring tooth 1124 when the physician applies a proximally directed force in the first axial direction (proximal direction) to the second actuation member 1110 to effect movement of the expansion and locking mechanism 1100 towards the axially compressed position (and thereby allow the valve 1200 to move towards the radially expanded position). In some embodiments, the free end 1127 can have a sharpened edge that can increase the frictional resistance between the free end 1127 and the rod 1114 when a distally directed force is applied to the rod 1114 and the second actuation member 1110.

In some embodiments, as described above, the spring tooth 1124 is configured to provide a constant holding force to the rod 1114 and/or the second actuation member 1110. However, in other embodiments, the spring tooth 1124 may be configured to vary the holding force exerted on the rod 1114 and/or the second actuation member 1110 while still continuously maintaining contact with the rod 1114 and/or the second actuation member 1110. As just one example, the biasing member 1126 and/or spring tooth 1124 may be configured to deform, deflect, and/or bend away from the opening 1120 (downwards in FIG. 20) when the physician pulls on the second actuation member 1110 (while still continuously maintaining contact with the rod 1114 and/or the second actuation member 1110) to reduce the holding force exerted on the rod 1114 and/or second actuation member 1110 and thereby make it easier for the physician to expand the prosthetic valve 1200 (i.e., require less force to pull (e.g., slide) the rod 1114 and/or second actuation member 1110 through the opening 1120 in the proximal member 1104, past the spring tooth 1124). However, in such examples, when the physician pauses the valve expansion (e.g., stops pulling on the second actuation member 1110) the spring tooth 1124 may automatically and/or passively return to the extended position to increase the holding force to ensure that the distal member 1102 cannot move distally in the second axial direction (to the left in FIG. 20) back towards the elongated position. Thus, by only allowing the rod 1114 and/or second actuation member 1110 to move in the first axial direction relative to the proximal member 1104, the spring tooth 1124 may continuously lock the valve 1200 in place at any valve diameter during the expansion process.

As explained above, when the expansion and locking mechanism 1100 is in the axially extended position, (FIG. 18A) the prosthetic valve 1200 is in the radially compressed position (FIG. 19A). In the illustrated embodiment, when in the axially extended position, the second actuation member 1110 of the actuation assembly 1106 can extend distally through the proximal member 1104, past the locking element 1122. However, as the physician pulls the second actuation member 1110 proximally towards the handle of the delivery apparatus, the second actuation member 1110 withdraws from the proximal member 1104 and the rod 1114 moves towards and through the proximal member 1104 (downwards in FIG. 19A). The rod 1114 is long enough such that in the axially compressed position (FIG. 18B), where the prosthetic valve 1200 is in the radially expanded position (FIG. 19B), the rod 1114 extends proximally through the proximal member 1104, at least past the locking element 1122 so that the locking element 1122 frictionally engages the rod 1114 to hold the prosthetic valve 1200 in the radially expanded position. In some examples, the rod 1114 may be sized such that it does not extend proximally past the proximal end 1206 of the prosthetic valve 1200 by more than a threshold amount, such as by more than one quarter of the axial length of the radially expanded prosthetic valve 1200. In alternative embodiments, the rod 1114 can be sized such that it extends proximally through the proximal member 1104 when the prosthetic valve 1200 is in the radially compressed and expanded states such that the locking element 1122 can remain engaged with the rod 1114 at every diameter of the prosthetic valve 1200 between the fully radially compressed and fully radially expanded states and the second actuation member 1110 is always positioned proximal to the proximal member 1104.

The rod 1114 and the second actuation member 1110 are sized, shaped, and/or otherwise configured to engage and be held by the locking element 1122, such that the rod 1114 and the second actuation member 1110 cannot move in the second axial direction (towards the extended position shown in FIG. 18A). For example, the rod 1114 and the second actuation member 1110 may be thick enough to frictionally engage with the locking element 1122 of the proximal member 1104. In some embodiments, such as the embodiments shown in FIGS. 18A-19B, the rod 1114 and second actuation member 1110 may have the same or similar cross-sectional area, thickness, and/or shape, such that they are configured to require the same or similar amount of force to be moved in the first axial direction towards the handle of the delivery apparatus.

However, in other embodiments, the second actuation member 1110 and the rod 1114 may have different cross-sectional areas, thickness, and/or shapes and may require different amounts of force to be moved proximally (e.g., pulled) towards the handle of the delivery apparatus. As just one example, the second actuation member 1110 may be thinner than the rod 1114. In such examples, it may be easier to pull the second actuation member 1110 through the proximal member 1104 than the rod 1114. As such, when the thicker rod 1114 reaches the locking element 1122, the second actuation member 1110 may become harder to pull (i.e., the physician may have to provide more force to continue expanding the prosthetic valve 1200), thereby providing an indication to the physician that the prosthetic valve is nearing its radially expanded position. In this way, the expansion and locking mechanism 1100 may provide the physician with tactile feedback that provides an indication of the expansion progress (i.e., the current valve diameter, how expanded the valve is, how much more the valve needs to be expanded before it reaches the radially expanded position, etc.).

In some embodiments, the rod 1114 and/or the second actuation member 1110 may have a uniform and/or consistent thickness, cross-sectional area, and/or shape. In other embodiments, the rod 1114 and/or the second actuation member 1110 may have a non-uniform and/or variable thickness, cross-sectional area, and/or shape that may provide the physician with even more granular indications of the current diameter of the valve (i.e., how expanded the valve is), and thus, more precise control over the expansion process. For example, the rod 1114 may be tapered such that it is initially easier to pull through the locking element 1122 when it first enters the proximal member 1104 but may become increasingly more difficult to pull through the locking element due to an increase in its thickness. This may help ensure a smoother end to the expansion process and may prevent excessive pulling forces from over-expanding the valve 1200 and/or from jarring and/or displacing the valve 1200 relative to the tissue. As one such example, the rod 1114 may become thick enough and/or may include a stopping member (e.g., flange) to prevent the valve from radially expanding past a predetermined point. In this way, the physician may have more precise control over the final stages of the expansion process and may not over-expand the valve.

The proximal member 1104 also may include an attachment member 1126 that is configured to be coupled to the frame 1202 (e.g., at the proximal junction 1214 or proximal apex 1212). For example, the attachment member 1126 may extend through an opening in the frame 1202 to help secure the distal member 1102 to the frame 1202. In some such examples, a mating mechanical fastener (e.g., nut) may be coupled to the end of the attachment member 1126 on the opposite side of the frame 1202 to prevent the attachment member 1126 from pulling out of the opening in the frame 1202.

As introduced above, the rod 1114 and the second actuation member 1110 are removably coupled to one another such that the second actuation member 1110 is configured to pull the rod 1114 with it, through the proximal member 1104, during expansion of the prosthetic valve 1200. Specifically, because of its removable coupling to the distal member 1102 of the expansion and locking mechanism 1100, the second actuation member 1110 pulls the distal member 1102 with it as it moves (e.g., slides) proximally relative to (e.g., through) the first actuation member 1108 towards the handle of the delivery apparatus, thereby axially foreshortening and radially expanding the valve 1200.

Once the prosthetic valve 1200 is expanded, the second actuation member 1110 is configured to be decoupled and/or detached from the rod 1114 so that the actuation assembly 1106 and delivery apparatus can be removed from the patient. In some embodiments, the rod 1114 and the second actuation member 1110 are coupled to one another at a proximal end 1128 of the rod 1114 and a distal end 1130 of the second actuation member 1110. In some embodiments, the rod 1114 may include a first removable coupling member 1132 and the second actuation member 1110 may include a second removable coupling member 1134 that is configured to be removably coupled to the first removable coupling member 1132 of the rod 1114. The removable coupling members 1132, 1134 may comprise mechanical, magnetic, and/or other suitable removable coupling means. For example, in the embodiment illustrated in FIGS. 18A-20, the removable coupling members 1132, 1134 may comprise a threaded engagement. Specifically, the coupling member 1132 of the rod 1114 may comprise a male thread and the coupling member 1134 of the second actuation member 1110 may include a mating female thread that is configured to be threaded onto the threads of the coupling member 1132. However, in other embodiments, the removable coupling members 1132, 1134 may comprise alternative types of mechanical coupling arrangements, such as hook and loop fasteners, snap-fit coupling arrangements, latches, etc.

In this way, a physician may expand the prosthetic valve 1200 as desired without the risk of valve collapse. Specifically, because the locking element 1122 prevents the rod 1114 and/or second actuation member 1110 (whichever is positioned within the opening 1120 of the proximal member 1104 where the locking element 1122 is located) from moving distally towards the extended position, the prosthetic valve 1200 cannot radially compress once it has been radially expanded. Further, because the locking element 1122 is self-locking (i.e., it continuously holds the rod 1114 and/or second actuation member 1110 in place), the physician can expand the prosthetic valve 1200 in any desired manner. For example, the physician can continuously and smoothly expand the prosthetic valve if desired. As another example, the physician can expand the prosthetic valve in a series of pulses if desired. As yet another example, the physician can partially expand the prosthetic valve, pause the expansion to evaluate operating conditions, and then resume the expansion.

In operation, the physician can advance the valve 1200 to the implantation site via the delivery apparatus. For example, the physician can advance the delivery apparatus through the patient's vasculature by gripping the handle of the delivery apparatus and providing a distally directed force (e.g., a pushing force). Once at the implantation site, the physician can deploy the prosthetic valve 1200 from the delivery sheath (e.g., delivery sheath 116) by, for example, adjusting a first control mechanism (e.g., first knob 110) included on the handle. When the prosthetic valve 1200 is in the desired location at the implantation site, the physician can apply the distally directed force to the proximal member 1104 via the first actuation member 1108 and/or can apply the proximally directed force to the distal member 1102 via the second actuation member 1110 to radially expand the valve 1200. As one example, the physician can pull directly on the second actuation member 1110 to apply the proximally directed force to the distal member 1102. In other examples, the physician can adjust a second control mechanism (e.g., second knob 112) included on the handle of the delivery apparatus to apply the proximally directed force to the second actuation member 1110. During the radial expansion of the prosthetic valve 1200, the locking element 1122 continuously locks the expansion and locking mechanism 1100 to prevent the prosthetic valve from retracting towards a more radially compressed position. When the prosthetic valve is expanded to a desired position, the physician can detach the second actuation member 1110 from the distal member 1102 by, for example, unscrewing the second actuation member 1110 from the distal member 1102, and can then remove the delivery apparatus from the patient.

In FIGS. 19A and 19B, roughly only half of the valve frame 1202 is shown for simplicity and clarity. However, it should be appreciated that the valve frame 1202 is substantially cylindrical and encloses the lumen 1218, like the frames shown in FIGS. 1, 3, 5-6, 9A, and 13. Further, like in FIGS. 3, 5-6, 9A, and 13, the valvular structure of the valve 1200 is omitted for simplicity and clarity, but it should be appreciated that the valve 1200 may include the same and/or similar valvular structure shown in FIG. 1 (valvular structure 18).

Further, although only one expansion and locking mechanism 1100 is shown in FIGS. 19A-19B, it should be appreciated that the prosthetic valve 1200 may include more than one expansion and locking mechanism 1100 in other examples. For example, the prosthetic valve 1200 may include two expansion and locking mechanisms 1100, three expansion and locking mechanisms 1100, and/or four or more expansion and locking mechanisms 1100. When more than one expansion and locking mechanism 1100 is included with the prosthetic valve 1200, the expansion and locking mechanisms 1100 may be evenly or unevenly circumferentially spaced around the frame 1202. Similarly, the delivery apparatus can have a number of actuation assemblies 1106 equal to the number of expansion and locking mechanisms 1100. The delivery apparatus can be configured to actuate each of the actuation assemblies 1106 individually and/or simultaneously.

Further, it should be appreciated that the distal end 1204 of the prosthetic valve 1200 is positioned farther from the handle of the delivery apparatus than the proximal end 1206 of the prosthetic valve 1200. That is, the distal end 1204 is configured to be positioned deeper within the patient's vasculature. In some examples, the distal end 1204 of the prosthetic valve 1200 may be the inflow end of the prosthetic valve 1200 and the proximal end 1206 of the prosthetic valve 1200 may be the outflow end of the valve 1200, such as when the prosthetic valve 1200 is configured to replace a native aortic valve and the prosthetic valve 1200 is delivered to the native aortic valve via a retrograde, transfemoral delivery approach (e.g., through a femoral artery and the aorta). However, in other embodiments, the distal end 1204 may be the outflow end of the prosthetic valve 1200 and the proximal end 1206 may be the inflow end of the prosthetic valve 1200, such as when the prosthetic valve 1200 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

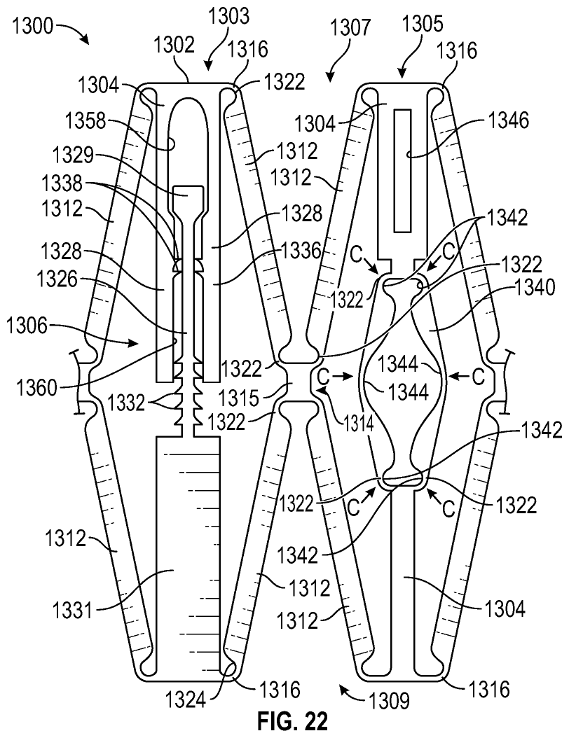

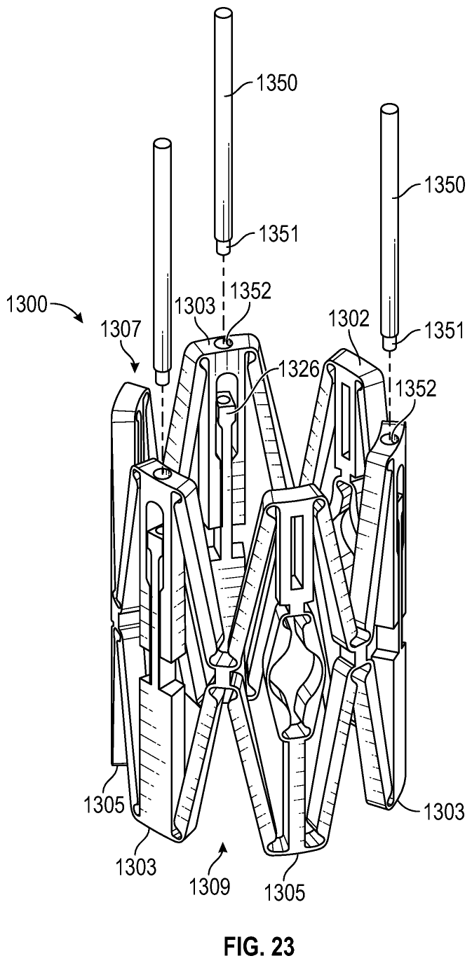

FIGS. 22-25 illustrate an exemplary embodiment of a prosthetic heart valve 1300 having a frame 1302. The prosthetic valve can include a valvular structure comprising a plurality of leaflets (such as valvular structure 18 comprising leaflets 20) and inner and/or outer skirts, as previously described, though these components are omitted for purposes of illustration. The prosthetic valve 1300 can be similar to prosthetic valve 200, except that prosthetic valve 1300 can comprise a slightly different structure than the prosthetic valve 200 and can be expanded by the delivery apparatus in a different manner. As far differences in structure, the prosthetic valve 1300 can include additional cells not shown for valve 200. Specifically, unlike valve 200, which can include both a commissure window and an expansion and locking mechanism at or in the same post and/or cell, the valve 1300 can include separate cells and/or posts for the commissure windows and the expansion and locking mechanisms. As for differences in how the valves 200 and 1300 are expanded and/or actuated, unlike the valve 200 which can be radially expanded by applying a distally directed (e.g., pushing) force to the inner members of the expansion and locking mechanisms, the valve 1300 can be radially expanded by applying a proximally directed (e.g., pulling) force to the inner members of the expansion and locking mechanisms of the valve 1300.

Components of valve 1300 that are the same as and/or similar to components of valve 200 are similarly numbered for convenience. For example, frame 1302, posts 1304, expansion and locking mechanisms 1306, links 1312, compliant joints 1316, and C-shaped cutouts 1324 of valve 1300 can correspond to frame 202, posts 204, expansion and locking mechanisms 206, links 212, compliant joints 216, and C-shaped cutouts 224 of valve 200, respectively. For conciseness, these similarly numbered components may not be re-introduced or otherwise discussed again in the description of FIGS. 22-25 herein.

In the illustrated embodiment of FIGS. 22-25, the frame 1302 includes a first set of cells 1303 (also referred to herein as “actuation cells 1303”) that include the expansion and locking mechanisms 1306 and a second set of cells 1305 (also referred to herein as “commissure cells”) that can include the commissure windows 1346. Each of the cells 1303, 1305 is formed and/or defined by the four links or struts 212 that directly couple to a given post 1304.

The frame 1302 further includes a proximal end 1307 and a distal end 1309, where the proximal end 1307 is configured to be positioned closer to the handle of a delivery apparatus than the distal end 1309. In some examples, the proximal end 1307 can be an outflow end of the valve 1300, such as when the prosthetic valve 1300 is configured to replace a native aortic valve and the prosthetic valve 1300 is delivered to the native aortic valve via a retrograde, transfemoral delivery approach (e.g., through a femoral artery and the aorta). However, in other embodiments, the distal end 1309 may be the outflow end of the prosthetic valve 1300 and the proximal end 1307 may be the inflow end of the prosthetic valve 1300, such as when the prosthetic valve 1300 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

Like the frame 202 of prosthetic valve 200, the frame 1302 can include compliant joints 1316 at or near the proximal end 1307 and the distal end 1309 that connect (e.g., pivotably couple) each of the links 1312 with one of the posts 1304. Further, towards the axial midsection of the valve 1300, the frame 1302 can include a four-way joint or interconnect 1314, such as interconnect 750, including a hub 1315 that connects (e.g., pivotably couples) four of the links 1312 between a cell 1303 and a cell 1305. Thus, one of the cells 1303, 1305 is included between adjacent interconnects 1314. Each of the joints 1316 and/or interconnects 1314 can include thin, flexible neck portions 1322 that permit the links to pivot relative to the posts 1304 and/or hubs 1315 as the valve 1300 radially expands and/or compresses. As described above, the joints 1316 and/or interconnects 1314 can include one or more of the different types of joints and/or hinges described above (e.g., leaf type hinge 500, beam type hinge 600, flat spring type hinge 700, etc.). As also described above, one or more of the joints 1316 and/or one or more of the interconnects 1314 can help lock the frame 1302 in a radially expanded state by, for example, being fully or partially plastic.

Additionally or alternatively, one or more of the expansion and locking mechanisms 1306 can help lock the frame 1302 in a radially expanded state. For example, as shown in FIG. 22, the inner member 1326 (which can also be referred to as “distal member 1326” and/or “actuation member 1326”) and the outer members 1328 (which can also be referred to as the “proximal member 1328”) of one or more of the expansion and locking mechanisms 1306 optionally can include locking teeth 1332 and 1338, respectively, that can engage with one another to prevent the inner member 1326 from moving distally relative to the outer members 1328, thereby holding the valve 1300 in a radially expanded state and preventing radially compression of the valve. The outer members 1328 can temporarily deflect away from one another (circumferentially) to allow the teeth 1332 of the inner member 1326 to slide past the teeth 1338 of the outer members 1328 and can return to their original state (e.g., shown in FIG. 22) when the teeth 1332 of the inner member 1326 clear the teeth 1328 of the outer members 1328 to ensure that the teeth 1332 of the inner member 1326 cannot slip backwards (distally) past the teeth 1338, thereby locking the frame 1302 in a radially expanded state. However, in other examples, the inner and outer members 1326, 1328 can include other types of locking mechanisms. In yet further examples, the inner and outer members 1326, 1328 can entirely omit locking mechanisms. In such examples, the inner and outer members 1326, 1328 can move freely relative to one another without locking and can thus permit unrestricted radial expansion and/or compression of the valve 1300 for a range of valve diameters.

Additionally or alternatively, the posts 1304 of the second set of cells 1305 can include internal support cells 1340, one or more of which can help lock the frame 1302 in a radially expanded state. For example, one or more of the support cells 1340 can be fully or partially plastic, like the joints 1316 and/or interconnects 1314, such that they are elastic for narrower valve diameters but plastic at wider valve diameters to help lock the valve 1300 in one or more radially expanded states. However, in other examples, one or more of the support cells 1340 can be fully elastic and may not provide any locking functionality. Additionally or alternatively, one or more of the joints 1316 and/or the interconnects 1314 can be fully elastic. The support cells 1340 can bend and/or otherwise deform at corners C via flexible joints 1342, 1344. The gaps at joints 1342 can widen as the frame 1302 radially expands, while the gaps at joints 1344 can narrow as the frame 1302 radially expands.

In this way one or more of the joints 1316, the interconnects 1314, the support cells 1340, and/or the expansion and locking mechanisms 1306 can help lock the frame 1302 in a radially expanded state.

Like valve 200, the distal and proximal members 1326, 1328 can move (e.g., slide) axially relative to one another to radially expand and/or compress the valve 1300. In the example shown in FIGS. 22-25, the distal member 1326 is configured as the inner member and the proximal member 1328 is configured as two outer members 1328, wherein the distal member 1326 can slide axially within the two outer members 1328. However, it should be appreciated that this configuration can be reversed such that the distal member 1326 is configured as the outer members, wherein the proximal member 1328 can slide axially within the distal member 1326. In other examples, the proximal and distal members 1326, 1328 can include any number of overlapping prongs and/or arms that can move axially relative to one another.

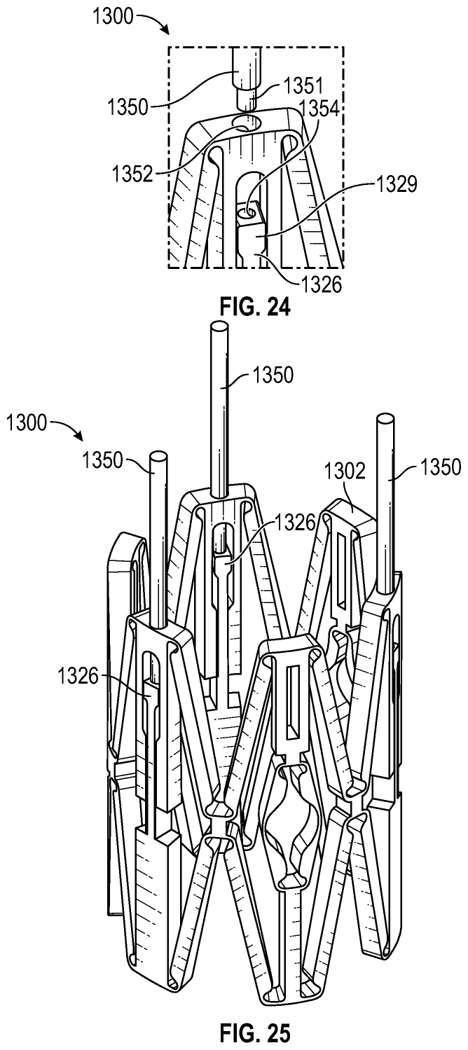

The distal member 1326 can include a base portion 1331 that can be wider than the rest of the distal member 1326. The distal member 1326 also can include a bulbous or widened end portion 1329 that is configured to couple to an actuation member 1350 (similar to actuation member 124) of a delivery apparatus, such as the delivery apparatus 100 of FIG. 2. Specifically, as shown in FIGS. 23-24, the frame 1302 can include axially extending channels or apertures 1352 that are configured to receive the actuation members 1350 of the delivery apparatus, and the end portions 1329 of the distal members 1326 can include axially extending channels or apertures 1354 that are configured to receive and/or couple to a distal end portion 1351 of the actuation members 1350. As one example, the distal end portion 1351 can be threaded and can be screwed into and/or out of the channels 1354 to couple and/or decouple the actuation members 1350 from the distal members 1326 of the frame 1302. However, other releasable mechanical connections can be used such as snap fit and/or friction fit arrangements, latches, clasps, pins, etc. Thus, the actuation members 1350 are configured to extend through the apertures 1352 in the proximal end of the frame 1302 and into the apertures 1354 in the distal members 1326. The apertures 1354 can extend axially through the posts 1304 from the proximal end 1307 of the frame 1302 to a channel 1358 in the posts 1304 that is formed between the outer members 1328.

In operation, the actuation members 1350 can be moved axially through the channels 1352 while a sleeve or support tube (e.g., support tubes 122 or 1108) or other structure (not shown) of the delivery apparatus can engage and/or abut the proximal end of the valve 1300 and simultaneously provide a countervailing force to help expand and/or compress the valve 1300 while keeping the valve 1300 in place relative to the surrounding tissue. For example, the actuation members 1350 can be moved proximally (e.g., pulled) through the channels 1352 in the frame 1302, such as by a physician actuating a control mechanism of the delivery apparatus (e.g., pulling a string that is connected to the actuation members 1350, turning one or more knobs that can be included on a handle of the delivery apparatus, etc.), while the sleeve can provide a distally directed force to the proximal end 1307 of the valve 1300 to radially expand the valve 1300. Since, the actuation members 1350 are releasably coupled to the distal members 1326, the distal members 1326 move axially with the actuation members 1350 when they are coupled thereto. In this way, the distal members 1326 can move axially towards the proximal end 1307 of the valve 1300 to cause radial expansion of the valve 1300 and can move axially towards the distal end 1309 of the valve 1300 to cause radial compression of the valve 1300. When the valve is fully radially expanded, the actuation members 1350 can be decoupled from the distal members 1326 (e.g., by unscrewing them from the distal members 1326) and then the delivery apparatus can be removed from the patient.

The distal and proximal members 1326, 1328 can move axially relative to one another in both directions. However, in examples where the members 1326, 1328 include a locking mechanism (e.g., locking teeth 1332, 1338), once the frame 1302 is radially expanded to a point where the locking teeth 1332, 1338 engage with one another, the frame 1302 cannot be re-compressed and can only be further radially expanded. The number and position of the teeth 1332, 1338 can be adjusted depending on the valve diameters at which locking is desired. For example, the teeth 1332 of the distal member 1326 can be positioned closer to the widened end portion 1329 of the distal member 1326 and/or the teeth 1338 of the proximal member 1328 can be positioned closer to the distal end of the pawls 1336 to provide locking at a wider range of valve diameters, and vice versa. In examples where the members 1326, 1328 do not include teeth or other locking mechanisms, they may be free to move axially in both the proximal and distal axial directions relative to one another.

That said, the widened end portion 1329 can prevent the distal member 1326 from moving distally relative to the proximal members 1328 past a certain point. Specifically, widened end portion 1329 may only be able to move within the channel 1358 of the proximal members 1328 and may be too wide to pass through a narrower channel 1360 of the proximal members 1328 that include the teeth 1338. Additionally or alternatively, the base portion 1331 of the distal member can prevent the distal member 1326 from moving proximally relative to the proximal members 1328 past a certain point. Specifically, the base portion 1331 can be too wide to pass through the narrower channel 1360 of the proximal members 1328 and as such, the base portion 1331 can prevent further radial expansion of the valve 1300 when it contacts and/or engages the proximal members 1328. In this way, the widened end portion 1329 and/or base portion 1331 of the distal member 1326 can constrain the valve 1300 to a certain range of valve diameters even when the members 1326, 1328 do not include any locking mechanisms.

The support cells 1340 can be positioned more proximate the distal end 1309 of the valve 1300 than the commissure windows 1346, such as in the axial mid-section of the posts 1304. As such, in some examples, the support cells 1340 can be substantially axially aligned with the interconnects 1314.

In the example shown in FIGS. 23 and 25, the first set of cells 1303 can include three cells 1303 and the second set of cells 1305 can include three cells 1305, wherein the cells are positioned in an alternating order circumferentially around the frame 1302, such that two of the cells 1303 are positioned adjacently on either side of each of the cells 1305, and vice versa. Accordingly, the frame 1302 includes three expansion and locking mechanism 1306, three support cells 1340, three commissure windows 1346 and three channels 1352. However, it should be appreciated that the frame 1302 can include more or less than three of each of these components, and/or the cells 1303, 1305 can be arranged in different orders. Further, not all of the cells 1305 have to include both a commissure window 1346 and an internal support cell 1340. In some examples, the cells 1305 can include one or the other. Further, in some examples, the frame 1302 can include additional cells in which the posts 1304 do not contain any of the expansion and locking mechanism 1306, the commissure windows 1346, and the support cells 1340. Further, although three actuation members 1350 are shown in FIGS. 23 and 25, other numbers of actuation members 1350 can be used to radially expand and/or compress the valve 1300.

In this way, the valve 1300 can be radially expanded through a range of valve diameters until the locking mechanisms are engaged (e.g., the locking teeth 1332, 1338 engage with one another and/or one or more of the compliant joints 1316, the interconnects 1314, and/or the support cells 1340 become plastically deformable). Once the valve 1300 is radially expanded to a threshold valve diameter (the valve diameter at which the locking mechanisms become engaged), the valve 1300 may no longer be able to be radially re-compressed and can be irreversibly held in at least a partially radially expanded state. However, in other examples, especially where locking teeth are not included and only the plastically deformable joints are used to help lock the valve in a radially expanded state, the valve 1300 can still be re-compressed if a sufficient compressive force is applied. In still further example, the valve 1300 can include an unlocking mechanism that is configured to disengage the locking mechanism (e.g., locking teeth) to permit the valve 1300 to be radially re-compressed even after it has been radially expanded beyond the threshold valve diameter.

As discussed above, this threshold valve diameter at which the valve 1300 becomes self-locking can be selected by, for example, adjusting the position of the locking teeth 1332, 1338 and/or the elasticity and/or plasticity of the compliant joints, 1316, the interconnects 1314, and/or the support cells 1340. In some examples, this threshold valve diameter at which the valve becomes locked can be at least 1.2, at least 1.5, at least 2, at least 2.5, at least 3, at most 50 times, at most 25 times, at most 10 times and/or at most 5 times the diameter of valve 1300 when the valve 1300 is in the radially compressed state (e.g., FIG. 4). In some examples, where the valve 1300 includes multiple locking mechanisms (e.g., where the valve 1300 includes the teeth 1332, 1338 and where one or more of the joints 1316, interconnects 1314, and/or cells 1340 are plastically deformable), the various locking mechanisms can engage at the same or different valve diameters.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic device, comprising:
a radially compressible and expandable frame, comprising:
   a plurality of posts extending axially from a proximal end of the frame to an opposite distal end of the frame, wherein each post of a subset of the plurality of posts includes a proximal member and a distal member that are axially movable relative to one another to radially expand and/or radially compress the frame, wherein the proximal member comprises:
      a channel that receives the distal member and within which the distal member is configured to axially slide relative to the proximal member to radially expand and/or compress the prosthetic device; and
      an axially extending aperture that extends distally from the proximal end of the frame to the channel, wherein the axially extending aperture is configured to receive an actuation member of a delivery apparatus; and
   wherein the distal member is configured to be removably coupled to the actuation member of the delivery apparatus.

Example 2. The prosthetic device of any example herein, particularly example 1, wherein the distal member comprises an axially extending aperture that is configured to receive the actuation member of the delivery apparatus.

Example 3. The prosthetic device of any example herein, particularly example 1 or example 2, wherein the distal member is configured to be removably coupled to the actuation member via a threaded connection.

Example 4. The prosthetic device of any example herein, particularly any one of examples 1-3, wherein the proximal and distal members include a locking mechanism that is configured to prevent radial compression of the prosthetic device at prosthetic device diameters greater than a threshold prosthetic device diameter.

Example 5. The prosthetic device of any example herein, particularly example 4, wherein the locking mechanism comprises interlocking teeth that are included on the proximal member and the distal member.

Example 6. The prosthetic device of any example herein, particularly any one of examples 1-5, wherein the subset of the plurality of posts is a first subset of posts, and wherein the plurality of posts further comprise a second subset of posts, wherein the second subset of posts is distinct from the first subset of posts in that the posts of the second subset of posts contain different structures than the posts of the first subset of posts.

Example 7. The prosthetic device of any example herein, particularly example 6, wherein each of one or more posts of the second subset of posts comprises a commissure window that extends radially through each post.

Example 9. The prosthetic device of any example herein, particularly example 6 or example 7, wherein each of one or more posts of the second subset of posts comprises an internal support cell that is configured to deform as the prosthetic device radially compresses and/or radially expands.

Example 9. The prosthetic device of any example herein, particularly example 8, wherein at least one internal support cell is configured to undergo plastic deformation at a prosthetic device diameter equal to or greater than a/the threshold prosthetic device diameter to help prevent radial compression of the prosthetic device at prosthetic device diameters greater than the threshold prosthetic device diameter.

Example 10. The prosthetic device of any example herein, particularly any one of examples 7-9, wherein the at least one internal support cell comprises compliant joints that are configured to deflect when the prosthetic device radially expands and/or radially compresses.

Example 11. The prosthetic device of any example herein, particularly any one of examples 1-10, wherein the proximal end is an outflow end of frame and the distal end is an inflow end of the frame.

Example 12. The prosthetic device of any example herein, particularly any one of examples 1-11, further comprising a plurality of links that extend circumferentially between adjacent posts.

Example 13. The prosthetic device of any example herein, particularly example 12, further comprising a plurality of first compliant joints that each pivotably couples one of the links to one of the posts.

Example 14. The prosthetic device of any example herein, particularly example 13, further comprising a plurality of interconnects positioned circumferentially between the plurality of posts and comprising a plurality of second compliant joints, wherein each of the plurality of interconnects pivotably couples four of the links.

Example 15. The prosthetic device of any example herein, particularly example 14, wherein one or more of the first compliant joints, one or more of the interconnects, and/or one or more of the at least one of the plurality of posts is/are configured to be plastically deformable to help prevent radial compression of the prosthetic device at prosthetic device diameters greater than or equal to a/the threshold prosthetic device diameter and permit radial compression of the prosthetic device at prosthetic device diameters less than the threshold prosthetic device diameter.

Example 16. The prosthetic device of any example herein, particularly example 15, wherein one or more of the first compliant joints, one or more of the interconnects, and/or one or more of the at least one of the plurality of posts reach their yield strength at the threshold prosthetic device diameter.

Example 17. A prosthetic device, comprising:
a radially compressible and expandable frame, comprising:
   a plurality of posts extending axially from a proximal end of the frame to an opposite distal end of the frame;
   a plurality of links that extend circumferentially between adjacent posts of the plurality of posts;
   a plurality of first compliant joints that each pivotably couples one of the links to one of the posts, wherein the plurality of first compliant joints comprise a plurality of first flexible neck portions; and
   a plurality of interconnects positioned circumferentially between the plurality of posts and comprising a plurality of second compliant joints, wherein each of the plurality of interconnects pivotably couples four of the links, and wherein the plurality of second compliant joints comprise a plurality of second flexible neck portions;
   wherein one or more of the first compliant joints and/or one or more of the second compliant joints elastically deform when the frame radially expands from a radially compressed state to a partially expanded state in an initial expansion range of the frame and then plastically deforms when the frame radially expands from the partially expanded state to a fully expanded state in a subsequent expansion range.

Example 18. The prosthetic device of any example herein, particularly example 17, wherein the first flexible neck portions and/or the second flexible neck portions are thinner than the links and/or the posts.

Example 19. The prosthetic device of any example herein, particularly example 17 or example 18, wherein the first flexible neck portions and/or the second flexible neck portions are as wide in a radial direction as the links and/or the posts.

Example 20. The prosthetic device of any example herein, particularly any one of examples 17-19, wherein the first flexible neck portions and/or the second flexible neck portions are wider than they are thick.

Example 21. The prosthetic device of any example herein, particularly any one of examples 17-20, wherein the first flexible neck portions and/or the second flexible neck portions are 1.2 times as wide as they are thick.

Example 22. The prosthetic device of any example herein, particularly any one of examples 17-21, wherein one or more of the first compliant joints and/or one or more of the second compliant joints are fully elastic, such that they remain elastic at all prosthetic device diameters.

Example 23. The prosthetic device of any example herein, particularly any one of examples 17-22, wherein one or more of the first compliant joints and/or one or more of the second compliant joints are partially plastic.

Example 24. The prosthetic device of any example herein, particularly any one of examples 17-23, wherein the plurality of interconnects are substantially X-shaped.

Example 25. An assembly, comprising:
a prosthetic device comprising a radially compressible and expandable frame, the frame comprising:
   a plurality of posts extending axially from a proximal end of the frame to an opposite distal end of the frame, wherein each post of a subset of the plurality of posts includes a proximal member and a distal member, wherein the proximal member comprises a channel within which the distal member is configured to axially slide relative to the proximal member to radially expand and/or compress the prosthetic device, and wherein the proximal member further comprises an axially extending aperture that extends distally from the proximal end of the frame to the channel;
   a plurality of links that extend circumferentially between adjacent posts of the plurality of posts;
   a plurality of first compliant joints, each of which pivotably couples one of the links to one of the posts; and
   a plurality of interconnects positioned circumferentially between the plurality of posts and comprising a plurality of second compliant joints, wherein each of the plurality of interconnects pivotable couples four of the links; and
a delivery apparatus comprising at least one actuation member configured to extend through the axially extending aperture of the proximal member and to removably couple to the distal member.

Example 26. The assembly of any example herein, particularly example 25, wherein the actuation member of the delivery apparatus and the distal member are configured to be removably coupled to one another via a threaded connection.

Example 27. The assembly of any example herein, particularly example 25 or example 26, wherein the actuation member is configured to be moved proximally to slide the distal member within the channel of the proximal member towards the proximal end of the frame to radially expand the prosthetic device.

Example 28. The assembly of any example herein, particularly any one of examples 25-27, wherein the subset of the plurality of posts is a first subset of posts, and wherein the plurality of posts further comprise a second subset of posts, wherein the second subset of posts is distinct from the first subset of posts in that the posts of the second subset of posts contain different structures than the posts of the first subset of posts.

Example 29. The assembly of any example herein, particularly example 28, wherein each of one or more posts of the second subset of posts comprises a commissure window that extends radially through each post.

Example 30. The assembly of any example herein, particularly example 28 or example 29, wherein each of one or more posts of the second subset of posts comprises an internal support cell that is configured to deform as the prosthetic device radially compresses and/or radially expands.

Example 31. The assembly of any example herein, particularly example 30, wherein at least one internal support cell is fully plastic and is configured to undergo plastic deformation at a threshold prosthetic device diameter to help prevent radial compression of the prosthetic device at prosthetic device diameters greater than the threshold prosthetic device diameter.

Example 32. The assembly of any example herein, particularly any one of examples 25-31, wherein each of the one or more of the first compliant joints is configured to undergo plastic deformation at a prosthetic device diameter equal to or greater than a/the threshold prosthetic device diameter to help prevent radial compression of the prosthetic device at prosthetic device diameters greater than the threshold prosthetic device diameter.

Example 33. The assembly of any example herein, particularly any one of examples 25-32, wherein each of the compliant joints of the one or more of the interconnects is configured to undergo plastic deformation at a prosthetic device diameter equal to or greater than a/the threshold prosthetic device diameter to help prevent radial compression of the prosthetic device at prosthetic device diameters greater than the threshold prosthetic device diameter.

Example 34. The assembly of any example herein, particularly any one of examples 25-33, wherein each post of the subset of the plurality of posts comprises a locking mechanism that is configured to prevent radial compression of the prosthetic device at prosthetic device diameters greater than a/the threshold prosthetic device diameter.

Example 35. The assembly of any example herein, particularly example 34, wherein the locking mechanism comprises interlocking teeth that are included on the proximal member and the distal member.

Example 36. A method, comprising:
radially expanding and/or compressing a prosthetic device by axially moving an actuation member of a delivery apparatus through an axially extending aperture of a post of a frame of the prosthetic device, wherein the aperture extends axially from a proximal end of the frame to a channel of a proximal member of the post within which a distal member of the post is configured to axially move, and wherein the actuation member is releasably coupled to the distal member.

Example 37. The method of any example herein, particularly example 36, further comprising decoupling the actuation member from the distal member and withdrawing the actuation member from the axially extending aperture after radially expanding the prosthetic device to a radially expanded state.

Example 38. The method of any example herein, particularly example 37, wherein the decoupling the actuation member from the distal member comprises unscrewing the actuation member from the distal member.

Example 39. The method of any example herein, particularly any one of examples 36-38, wherein the radially expanding and/or compressing the prosthetic device comprises applying a proximally directed force to the actuation member to radially expand the prosthetic device and/or applying a distally directed force to the actuation member to radially compress the prosthetic device.

Example 40. The method of any example herein, particularly example 39, wherein the applying the proximally and/or distally directed force(s) comprises adjusting a control mechanism included on a handle of the delivery apparatus.

Example 41. The method of any example herein, particularly any one of examples 36-40, further comprising locking the prosthetic device in a radially expanded state.

Example 42. The method of any example herein, particularly example 41, wherein the locking the prosthetic device in the radially expanded state comprises proximally moving the distal member within the channel of the proximal member until interlocking teeth of the proximal and distal members engage with one another.

Example 43. A prosthetic device, comprising:
a radially compressible and expandable frame, comprising:
   a plurality of posts extending axially from a first end of the frame to an opposite second end of the frame, wherein each post of a subset of the plurality of posts includes a first member and a second member that are axially movable relative to one another to radially expand and/or radially compress the frame;
   a plurality of links that extend circumferentially between adjacent posts;
   a plurality of first compliant joints, each of which pivotably couples one of the links to one of the posts; and
   a plurality of interconnects positioned circumferentially between the plurality of posts and comprising a plurality of second compliant joints, wherein each of the plurality of interconnects is pivotably coupled to four of the links; and
   wherein at least one of the plurality of posts comprises a locking mechanism that is configured to prevent radial compression of the prosthetic device at prosthetic device diameters greater than a threshold prosthetic device diameter and/or wherein one or more of the first compliant joints and/or one or more of the interconnects is/are configured to help prevent radial compression of the prosthetic device at prosthetic device diameters greater than or equal to the threshold prosthetic device diameter.

Example 44. The prosthetic device of any example herein, particularly example 43, wherein the subset of the plurality of posts is a first subset of posts, and wherein the plurality of posts further comprise a second subset of posts, wherein the second subset of posts is distinct from the first subset of posts in that the posts of the second subset of posts contain different structures than the posts of the first subset of posts.

Example 45. The prosthetic device of any example herein, particularly example 44, wherein each of one or more posts of the second subset of posts comprises a commissure window that extends radially through each post.

Example 46. The prosthetic device of any example herein, particularly example 44 or example 45, wherein each of one or more posts of the second subset of posts comprises an internal support cell that is configured to deform as the prosthetic device radially compresses and/or radially expands.

Example 47. The prosthetic device of any example herein, particularly example 46, wherein at least one internal support cell is fully plastic and is configured to undergo plastic deformation at a prosthetic device diameter equal to or greater than the threshold prosthetic device diameter to help prevent radial compression of the prosthetic device at prosthetic device diameters greater than the threshold prosthetic device diameter.

Example 48. The prosthetic device of any example herein, particularly any one of examples 43-47, wherein each of the one or more of the first compliant joints is configured to undergo plastic deformation at a prosthetic device diameter equal to or greater than the threshold prosthetic device diameter to help prevent radial compression of the prosthetic device at prosthetic device diameters greater than the threshold prosthetic device diameter.

Example 49. The prosthetic device of any example herein, particularly any one of examples 43-48, wherein each of the compliant joints of the one or more of the interconnects is configured to undergo plastic deformation at a prosthetic device diameter equal to or greater than the threshold prosthetic device diameter to help prevent radial compression of the prosthetic device at prosthetic device diameters greater than the threshold prosthetic device diameter.

Example 50. The prosthetic device of any example herein, particularly any one of examples 43-49, wherein the locking mechanism comprises interlocking teeth that are included on the first member and the second member.

Example 51. The prosthetic device of any example herein, particularly any one of examples 43-50, wherein one or more of the first compliant joints and/or the second compliant joints are fully elastic and do not plastically deform at any prosthetic device diameter

Example 52. The prosthetic device of any example herein, particularly any one of examples 43-51, wherein one or more of the first compliant joints and/or the second compliant joints are partially elastic and/or partially plastic.

Example 53. The prosthetic device of any example herein, particularly any one of examples 43-52, wherein each of the interconnects comprises a hub and four flexible neck portions, wherein each of the four flexible neck portions couples one of the links to the hub and is configured to bend to permit the link to which it is coupled to pivot relative to the hub to allow the prosthetic device to radially expand and/or compress.

Example 54. The prosthetic device of any example herein, particularly example 53, wherein the flexible neck portions are thinner in a radial direction than they are wide in a circumferential direction.

Example 55. The prosthetic device of any example herein, particularly example 54, wherein the flexible neck portions are at least 1.2 times as wide as they are thick.

Example 56. The prosthetic device of any example herein, particularly any one of examples 53-55, wherein the flexible neck portions are thinner and/or narrower than the links and/or the posts.

Example 57. The prosthetic device of any example herein, particularly any one of examples 43-56, wherein each of the plurality of first compliant joints comprises a flexible neck portion that is configured to bend to permit the link to which it is coupled to bend relative to the post to which it is coupled to allow the prosthetic device to radially expand and/or compress.

Example 58. The prosthetic device of any example herein, particularly example 57, wherein the flexible neck portion of each of the plurality of first compliant joints is narrower and/or thinner than the links and/or the posts.

Example 59. The prosthetic device of any example herein, particularly any one of examples 43-58, wherein one or more of the plurality of first compliant joints comprise a leaf type joint.

Example 60. The prosthetic device of any example herein, particularly any one of examples 43-59, wherein one or more of the plurality of first compliant joints comprise a beam type joint.

Example 61. The prosthetic device of any example herein, particularly any one of examples 43-60, wherein one or more of the plurality of first compliant joints comprise a flat spring type joint.

Example 62. The prosthetic device of any example herein, particularly any one of examples 43-61, wherein the first member comprises a channel that is configured to receive the second member, and wherein the second member is configured to slide axially within the channel of the first member.

Example 63. The prosthetic device of any example herein, particularly any one of examples 43-62, wherein the first member extends axially from the first end of the frame towards the second end of the frame, and wherein the second member extends axially from the second end of the frame towards the first end of the frame.

Example 64. The prosthetic device of any example herein, particularly any one of examples 43-63, wherein each post of the subset of the plurality of posts that includes a first member and a second member comprises an axially extending aperture that is configured to receive an actuation member of a delivery apparatus, wherein the aperture extends between the first end of the frame and a/the channel of the first member of the post.

Example 65. The prosthetic device of any example herein, particularly any one of examples 43-64, wherein the second member comprises an axially extending aperture that is configured to receive an/the actuation member of a/the delivery apparatus, wherein the aperture extends towards the second end of the frame from an end of the second member.

Example 66. The prosthetic device of any example herein, particularly any one of examples 43-65, wherein the first end is a proximal end of frame and the second end is a distal end of the frame, wherein the proximal end is configured to be positioned closer to a handle of a delivery apparatus than the distal end.

Example 67. The prosthetic device of any example herein, particularly any one of examples 43-66, wherein the first end of the frame is an outflow end of the prosthetic device and wherein the second end of the frame is an inflow end of the prosthetic device.

Example 68. A prosthetic device, comprising:
a frame that is radially expandable and compressible between a radially compressed state and a radially expanded state, the frame comprising:
   a plurality of posts extending axially from an inflow end of the frame to an outflow end;
   a plurality of links that extend circumferentially between adjacent posts of the plurality of posts; and
   a plurality of compliant joints that each pivotably couples one of the plurality of links to one of the plurality of posts; and
   wherein as the frame moves from the radially compressed state to the radially expanded state the compliant joints deflect circumferentially.

Example 69. The prosthetic device of any example herein, particularly example 68, wherein each compliant joint comprises a flexible neck portion defining a C-shaped cutout including a gap.

Example 70. prosthetic device of any example herein, particularly example 69, wherein the gap widens as the frame moves from the radially compressed state to the radially expanded state.

Example 71. The prosthetic device of any example herein, particularly any one of examples 68-70, wherein the frame comprises three posts.

Example 72. The prosthetic device of any example herein, particularly any one of examples 68-71, wherein each link has a first width and the flexible neck portion has a second width narrower than the first width.

Example 73. The prosthetic device of any example herein, particularly any one of examples 68-72, wherein each pair of adjacent posts is coupled together by four links.

Example 74. The prosthetic device of any example herein, particularly example 73, wherein the links are arranged in an X-shape.

Example 75. The prosthetic device of any example herein, particularly any one of examples 68-74, wherein each link comprises a first end portion and a second end portion and wherein each link is coupled to a post at the first end via a first compliant joint and coupled to an adjacent link at the second portion via a second compliant joint.

Example 76. The prosthetic device of any example herein, particularly example 75, wherein the first and second compliant joints are oriented in opposing directions.

Example 77. The prosthetic device of any example herein, particularly example 76, wherein the first compliant joint is oriented toward an inflow end of the frame and the second compliant joint is oriented toward an outflow end of the frame.

Example 78. The prosthetic device of any example herein, particularly any one of examples 68-77, wherein the compliant joints are flush with the links such that they do not protrude past a radially inner or outer surface of the links.

Example 79. The prosthetic device of any example herein, particularly any one of examples 68-78, wherein the links and posts define a circumferentially extending row of triangularly-shaped cells.

Example 80. The prosthetic device of any example herein, particularly any one of examples 68-79, wherein the frame is formed from a unitary piece of material.

Example 81. The prosthetic device of any example herein, particularly any one of examples 68-80, wherein each post comprises a commissure window extending through a thickness of the post.

Example 82. The prosthetic device of any example herein, particularly example 81, wherein the commissure window has a rectangular shape.

Example 83 The prosthetic device of any example herein, particularly example 81 or example 82, further comprising a valvular structure comprising a plurality of leaflets each including one or more tabs, wherein tabs of adjacent leaflets are disposed within the commissure window to couple the valvular structure to the frame.

Example 84. The prosthetic device of any example herein, particularly any one of examples 68-83, wherein one or more posts are configured as expansion and locking mechanisms comprising an inner member and one or more outer members.

Example 85. The prosthetic device of any example herein, particularly example 84, wherein the inner member extends from an outflow end portion of the frame toward an inflow end portion of the frame, and wherein the one or more outer members extend from the inflow end portion of the frame toward the outflow end portion.

Example 86. The prosthetic device of any example herein, particularly example 84 or example 85, wherein the inner member comprises a linear rack having a plurality of teeth arrayed along a length of the inner member, and wherein at least one outer member comprises a pawl configured to engage the rack to allow movement of the inner member relative to the outer members in a first direction and prevent movement of the inner member relative to the outer members in a second direction opposite the first direction.

Example 87. The prosthetic device of any example herein, particularly example 86, wherein the at least one pawl is biased toward the plurality of teeth.

Example 88. The prosthetic device of any example herein, particularly any one of examples 84-87, wherein the inner member comprises a first linear rack disposed on a first circumferential edge and a second linear rack disposed on a second circumferential edge.

Example 89. The prosthetic device of any example herein, particularly example 88, wherein each post comprises a first outer member including a first pawl configured to engage the first linear rack and a second outer member including a second pawl configured to engage the second linear rack.

Example 90. The prosthetic device of any example herein, particularly example 89, wherein the first and second outer members are disposed circumferentially on either side of the inner member.

Example 91. The prosthetic device of any example herein, particularly any one of examples 87-90, wherein the inner member comprises a toothless portion along which the pawls of the one or more outer members can slide without engagement.

Example 92. The prosthetic device of any example herein, particularly any one of examples 84-91, wherein the inner member comprises one or more stopping surfaces positioned to selectively abut an end portion of the one or more outer members to prevent distal movement of the inner member past a predetermined point.

Example 93. A prosthetic device, comprising:
a frame that is radially expandable and compressible between a radially compressed state and a radially expanded state, the frame comprising:
   a plurality of posts, each post comprising an axially extending first member and two axially extending second members, the first member comprising a plurality of teeth and the second members each comprising a locking tooth configured to engage the plurality of teeth to allow movement in a first direction to allow radial expansion of the frame and prevent movement in a second direction to prevent radial compression of the frame; and
   a plurality of links coupling adjacent posts to one another via one or more compliant joints, each compliant joint comprising a flexible neck portion configured to deflect circumferentially as the frame between the radially compressed state and the radially expanded state.

Example 94. The prosthetic device of any example herein, particularly example 93, wherein the frame is formed from a unitary piece of material.

Example 95. The prosthetic device of any example herein, particularly example 93 or example 94, wherein each link has a first width and the flexible neck portion has a second width narrower than the first width.

Example 96. The prosthetic device of any example herein, particularly any one of examples 93-95, wherein each pair of adjacent posts is coupled together by four links.

Example 97. The prosthetic device of any example herein, particularly 96, wherein the links are arranged in an X-shape.

Example 98. The prosthetic device of any example herein, particularly any one of examples 93-97, wherein each link comprises a first end portion and a second end portion and wherein each link is coupled to a post at the first end via a first compliant joint and coupled to an adjacent link at the second portion via a second compliant joint.

Example 99. The prosthetic device of any example herein, particularly example 98, wherein the first and second compliant joints are oriented in opposing directions.

Example 100. The prosthetic device of any example herein, particularly example 99, wherein the first compliant joint is oriented toward an inflow end of the frame and the second compliant joint is oriented toward an outflow end of the frame.

Example 101. The prosthetic device of any example herein, particularly any one of examples 93-100, wherein the compliant joints are flush with the links such that they do not protrude past a radially inner or radially outer surface of the links.

Example 102. The prosthetic device of any example herein, particularly any one of examples 93-101, wherein the links and posts define a circumferentially extending row of triangularly-shaped cells.

Example 103. The prosthetic device of any example herein, particularly any one of examples 93-102, wherein each post comprises a commissure window extending through a thickness of the post.

Example 104. The prosthetic device of any example herein, particularly example 103, wherein the commissure window has a rectangular shape.

Example 105. The prosthetic device of any example herein, particularly example 103 or example 104, further comprising a valvular structure comprising a plurality of leaflets each including one or more tabs, wherein tabs of adjacent leaflets are disposed within the commissure window to couple the valvular structure to the frame.

Example 106. A prosthetic device, comprising:

a frame that is radially expandable and compressible between a radially compressed state and a radially expanded state, the frame comprising a plurality of rigid links, each link comprising a first end portion coupled to a first adjacent link via a first compliant joint and a second end portion coupled to a second adjacent link via a second compliant joint, each compliant joint comprising a flexible neck portion configured to deflect circumferentially as the frame moves from the radially compressed state to the radially expanded state.

Example 107. The prosthetic device of any example herein, particularly example 106, wherein the frame is a first sub-frame and wherein the prosthetic device further comprises a second sub-frame disposed radially within and coupled to the first sub-frame.

Example 108. The prosthetic device of any example herein, particularly example 107, wherein the second sub-frame comprises a plurality of rigid links, each link comprising a first end portion coupled to a first adjacent link via a first compliant joint and a second end portion coupled to a second adjacent link via a second compliant joint, each compliant joint comprising each compliant joint comprising a flexible neck portion configured to deflect circumferentially as the frame moves from the radially compressed state to the radially expanded state.

Example 109. The prosthetic device of any example herein, particularly example 107 or example 108, wherein the first and second sub-frames are coupled together via fasteners extending through apertures in the links.

Example 110. The prosthetic device of any example herein, particularly any one of examples 106-109, further comprising one or more expansion and locking mechanisms, each expansion and locking mechanism comprising:
a first member coupled to the frame at a first location,
a second member coupled to the frame at a second location spaced apart from the first location, the second member extending at least partially into the first member, and
a locking member coupled to the first member, the locking member configured to engage the second member to allow movement in a first direction to allow radial expansion of the frame and prevent movement in a second direction to prevent radial compression of the frame.

Example 111. A prosthetic device, comprising:
a frame movable between a radially compressed state and a radially expanded state, the frame comprising:
   a first sub-frame and a second sub-frame disposed radially within the first sub-frame, each sub-frame comprising a set of rigid links, each link comprising a first end portion coupled to a first adjacent link via a first compliant joint and a second end portion coupled to a second adjacent link via a second compliant joint;
wherein each compliant joint comprises a flexible neck portion configured to deflect circumferentially as the frame moves from the radially compressed state to the radially expanded state; and
wherein the first and second sub-frames are coupled together via a plurality of fasteners.

Example 112. The prosthetic device of any example herein, particularly example 111, further comprising one or more expansion and locking mechanisms, each expansion and locking mechanism comprising
a first member coupled to the frame at a first location,
a second member coupled to the frame at a second location spaced apart from the first location, the second member extending at least partially into the first member, and
a locking member coupled to the first member, the locking member configured to engage the second member to allow movement in a first direction to allow radial expansion of the frame and prevent movement in a second direction to prevent radial compression of the frame.

Example 113. An assembly, comprising:
a prosthetic device comprising:
   a frame that is radially expandable and compressible between a radially compressed state and a radially expanded state, the frame comprising:
      a plurality of posts, one or more posts configured as an expansion and locking mechanism comprising an inner member including a linear rack having a plurality of teeth, and one or more outer members configured to engage the rack to allow movement in a first direction to allow radial expansion of the frame and prevent movement in a second direction to prevent radial compression of the frame; and
      a plurality of links configured to couple adjacent posts to one another via one or more compliant joints, each compliant joint configured to deflect circumferentially as the frame moves from the radially compressed state to the radially expanded state;
a delivery apparatus comprising:
   a handle;
   a first actuation member extending from the handle and coupled to an outflow end of the frame, the first actuation member configured to apply a distally directed force to the inner member;
   a second actuation member extending from the handle and coupled to an inflow end of the frame, the second actuation member configured to apply a proximally directed force to the one or more outer members;
wherein the prosthetic device is radially expandable from the radially compressed state to the radially expanded state upon application of at least one of the distally directed force and the proximally directed force to the prosthetic device via the first and second actuation members, respectively; and
wherein when the prosthetic device is in the radially expanded state the one or more outer members engage the rack to prevent compression of the frame.

Example 114. The assembly of any example herein, particularly example 113, wherein each compliant joint comprises a flexible neck portion defining a C-shaped cutout including a gap.

Example 115. The assembly of any example herein, particularly example 114, wherein the gap widens as the prosthetic device moves from the radially compressed state to the radially expanded state.

Example 116. The assembly of any example herein, particularly any one of examples 113-115, wherein each post comprises a commissure window extending through a thickness of the post.

Example 117. The assembly of any example herein, particularly example 116, wherein the commissure window has a rectangular shape.

Example 118. The assembly of any example herein, particularly example 116 or example 117, further comprising a valvular structure comprising a plurality of leaflets each including one or more tabs, wherein tabs of adjacent leaflets are disposed within the commissure window to couple the valvular structure to the frame.

Example 119. An assembly, comprising:
a prosthetic device comprising:
   a frame movable between a radially compressed state and a radially expanded state, the frame comprising:
      a first sub-frame and a second sub-frame, the second sub-frame disposed radially within the first sub-frame and coupled to the first sub-frame via a plurality of fasteners, each sub-frame comprising a set of links coupled to adjacent links via one or more compliant joints, each compliant joint comprising a flexible neck portion configured to deflect circumferentially as the frame moves from the radially compressed state to the radially expanded state;
   one or more expansion and locking mechanisms, each expansion and locking mechanism comprising a first member coupled to the frame at a first location, a second member coupled to the frame at a second location spaced apart from the first location, the second member extending at least partially into the first member, and a locking member coupled to the first member, the locking member configured to engage the second member to allow movement in a first direction to allow radial expansion of the frame and prevent movement in a second direction to prevent radial compression of the frame; and
a delivery apparatus comprising:
   a handle;
   a first actuation member extending from the handle and coupled to the first member, the first actuation member configured to apply a distally directed force to the first member; and
   a second actuation member extending from the handle and coupled to the second member, the second actuation member configured to apply a proximally directed force to the second member;
wherein the prosthetic device is radially expandable from the radially compressed state to the radially expanded state upon application of at least one of the distally directed force and the proximally directed force to the prosthetic device via the first and second actuation members, respectively; and
wherein when the prosthetic device is in the radially expanded state the locking member engages the second member to prevent compression of the frame.

Example 120. A method, comprising:
inserting a distal end of a delivery apparatus into the vasculature of a patient, the delivery apparatus releasably coupled to a prosthetic device including a frame that is radially expandable and compressible between a radially compressed state and a radially expanded state, the frame comprising:
   a plurality of posts, each post comprising an inner member and two outer members, the inner member comprising a plurality of teeth and the outer members each comprising a pawl configured to engage the plurality of teeth to allow movement in a first direction to allow radial expansion of the frame and prevent movement in a second direction to prevent radial compression of the frame; and
   a plurality of links configured to couple adjacent posts to one another via one or more compliant joints;
advancing the prosthetic device to a selected implantation site; and
moving at least one of the inner member distally and the outer members proximally to radially expand the prosthetic device such that the compliant joints deflect circumferentially and such that the pawls engage the plurality of teeth to lock the prosthetic device in a radially expanded state.

Example 121. A prosthetic device, comprising:
a radially expandable and compressible frame, the frame comprising:
   a plurality of posts, one or more posts being configured as expansion and locking mechanisms comprising:
      an axially extending first member; and
      an axially extending second member comprising a first side portion and a second side portion, the first side portion comprising a locking tooth configured to frictionally engage a surface of the first member to allow movement of the first member relative to the second member in a first direction to cause radial expansion of the frame and to prevent movement in a second direction to prevent radial compression of the frame; and
a plurality of links coupling adjacent posts to one another via one or more compliant joints.

Example 122. The prosthetic device of any example herein, particularly example 121, wherein the frame is formed from a unitary piece of material.

Example 123. The prosthetic device of any example herein, particularly example 121 or example 122, wherein the first side portion comprises a biasing member configured to bias the locking tooth against the first member.

Example 124. The prosthetic device of any example herein, particularly any one of examples 121-123, wherein the first member comprises a main portion, an elongated rod, and first and second angled surfaces tapering from the main portion to the elongated rod, the angled surfaces configured to prevent distal movement of the first member relative to the second member past a predetermined point.

Example 125. The prosthetic device of any example herein, particularly example 124, wherein the first and second side portions define a channel between them into which at least a portion of the elongated rod extends.

Example 126. The prosthetic device of any example herein, particularly any one of examples 121-125, wherein the posts are flush with the links such that they do not protrude past a radially inner or radially outer surface of the links.

Example 127. The prosthetic device of any example herein, particularly any one of examples 121-126, wherein each post comprises a commissure window extending through a thickness of the post.

Example 128. The prosthetic device of any example herein, particularly example 127, further comprising a valvular structure comprising a plurality of leaflets each including one or more tabs, wherein tabs of adjacent leaflets are disposed within the commissure window to couple the valvular structure to the frame.

Example 129. The prosthetic device of any example herein, particularly any one of examples 121-128, wherein the first member extends from an outflow end portion of the frame toward an inflow end portion of the frame, and wherein the second member extends from the inflow end portion of the frame toward the outflow end portion.

Example 130. The prosthetic device of any example herein, particularly any one of examples 121-129, wherein the first and second side portions are disposed circumferentially on either side of the first member.

Example 131. The prosthetic device of any example herein, particularly any one of examples 121-130, wherein each compliant joint comprises a flexible neck portion configured to deflect circumferentially as the frame moves from a radially compressed state to a radially expanded state.

Example 132. The prosthetic device of any example herein, particularly any one of examples 121-131, wherein the frame comprises three posts.

Example 133. A prosthetic device, comprising:
a radially expandable and compressible frame, the frame comprising:
   a plurality of posts, one or more posts being configured as expansion and locking mechanisms comprising:
      an axially extending first member;
      an axially extending second member comprising a first side portion and a second side portion defining a channel between them into which at least a portion of the first member extends; and
      a cap disposed over an outflow end portion of the second member, the cap comprising a biasing member configured to frictionally engage a surface of the first member to allow movement of the first member relative to the second member in a first direction to cause radial expansion of the frame and to prevent movement in a second direction to prevent radial compression of the frame; and
   a plurality of links coupling adjacent posts to one another via one or more compliant joints.

Example 134. The prosthetic device of any example herein, particularly example 133, wherein the frame except for the cap is formed from a unitary piece of metal.

Example 135. The prosthetic device of any example herein, particularly example 133 or example 134, wherein the first member comprises a main portion, an elongated rod, and first and second angled surfaces tapering from the main portion to the elongated rod, the angled surfaces configured to prevent distal movement of the first member relative to the second member past a predetermined point.

Example 136. The prosthetic device of any example herein, particularly any one of examples 133-135, wherein each post comprises a commissure window extending through a thickness of the post.

Example 137. The prosthetic device of any example herein, particularly example 136, further comprising a valvular structure comprising a plurality of leaflets each including one or more tabs, wherein tabs of adjacent leaflets are disposed within the commissure window to couple the valvular structure to the frame.

Example 138. The prosthetic device of any example herein, particularly any one of examples 133-137, wherein the first member extends from an outflow end portion of the frame toward an inflow end portion of the frame, and wherein the second member extends from the inflow end portion of the frame toward the outflow end portion.

Example 139. The prosthetic device of any example herein, particularly any one of examples 133-138, wherein the first and second side portions are disposed circumferentially on either side of the first member.

Example 140. The prosthetic device of any example herein, particularly any one of examples 133-139, wherein each compliant joint comprises a flexible neck portion configured to deflect circumferentially as the frame moves from a radially compressed state to a radially expanded state.

Example 141. The prosthetic device of any example herein, particularly any one of examples 133-140, wherein the frame comprises three posts.

Example 142. A prosthetic device, comprising:
a radially expandable and compressible frame, the frame comprising:
   a plurality of posts, one or more posts being configured as expansion and locking mechanisms comprising:
      an axially extending first member;
      an axially extending second member comprising a first side portion and a second side portion defining a channel between them into which at least a portion of the first member extends; and
      an annular cap disposed over an outflow end portion of the second member, the cap comprising an inner lumen extending along a length of the cap, the inner lumen tapering from a first diameter at an inflow end of the cap to a second, smaller diameter at an outflow end of the cap; and
   a plurality of links coupling adjacent posts to one another via one or more compliant joints;
wherein the cap is configured to urge the first and second side portions against the first member to prevent movement of the first member relative to the second member to prevent radial compression of the frame.

Example 143. The prosthetic device of any example herein, particularly example 142, wherein the frame except for the cap is formed from a unitary piece of metal.

Example 144. The prosthetic device of any example herein, particularly example 142 or example 143, wherein the cap is coupled to one or more links such that radial expansion of the frame causes distal movement of the cap.

Example 145. The prosthetic device of any example herein, particularly any one of examples 142-144, wherein the first member comprises a main portion, an elongated rod, and first and second angled surfaces tapering from the main portion to the elongated rod, the angled surfaces configured to prevent distal movement of the first member relative to the second member past a predetermined point.

Example 146. The prosthetic device of any example herein, particularly any one of examples 142-145, wherein each post comprises a commissure window extending through a thickness of the post.

Example 147. The prosthetic device of any example herein, particularly example 146, further comprising a valvular structure comprising a plurality of leaflets each including one or more tabs, wherein tabs of adjacent leaflets are disposed within the commissure window to couple the valvular structure to the frame.

Example 148. The prosthetic device of any example herein, particularly any one of examples 142-147, wherein the first member extends from an outflow end portion of the frame toward an inflow end portion of the frame, and wherein the second member extends from the inflow end portion of the frame toward the outflow end portion.

Example 149. The prosthetic device of any example herein, particularly any one of examples 142-148, wherein the first and second side portions are disposed circumferentially on either side of the first member.

Example The prosthetic device of any example herein, particularly any one of examples 142-149, wherein each compliant joint comprises a flexible neck portion configured to deflect circumferentially as the frame moves from a radially compressed state to a radially expanded state.

Example 151. The prosthetic device of any example herein, particularly any one of examples 142-150, wherein the frame comprises three posts.

Example 152. An assembly, comprising:
a prosthetic device comprising:
   a frame that is radially expandable and compressible between a radially compressed state and a radially expanded state, the frame comprising:
      a plurality of posts, one or more posts configured as expansion and locking mechanisms comprising an axially extending first member and an axially extending second member comprising a first side portion and a second side portion, the first side portion comprising a locking tooth configured to frictionally engage a surface of the first member to allow movement of the first member relative to the second member in a first direction to cause radial expansion of the frame and to prevent movement in a second direction to prevent radial compression of the frame; and
      a plurality of links coupling adjacent posts to one another via one or more compliant joints; and
a delivery apparatus comprising:
   a handle;
   a first actuation member extending from the handle and coupled to an outflow end of the frame, the first actuation member configured to apply a distally directed force to the first member; and
   a second actuation member extending from the handle and coupled to an inflow end of the frame, the second actuation member configured to apply a proximally directed force to the second member;
wherein the prosthetic device is radially expandable from the radially compressed state to the radially expanded state upon application of at least one of the distally directed force and the proximally directed force to the prosthetic device via the first and second actuation members, respectively; and
wherein when the prosthetic device is in the radially expanded state the locking tooth engages the first member to prevent compression of the frame.

Example 153. The assembly of any example herein, particularly example 152, wherein the frame is formed from a unitary piece of material.

Example 154. The assembly of any example herein, particularly example 152 or example 153, wherein the first side portion comprises a biasing member configured to bias the locking tooth against the first member.

Example 155. The assembly of any example herein, particularly any one of examples 152-154, wherein the first member comprises a main portion, an elongated rod, and first and second angled surfaces tapering from the main portion to the elongated rod, the angled surfaces configured to prevent distal movement of the first member relative to the second member past a predetermined point.

Example 156. The assembly of any example herein, particularly example 155, wherein the first and second side portions define a channel between them into which at least a portion of the elongated rod extends.

Example 157. The assembly of any example herein, particularly any one of examples 152-156, wherein the posts are flush with the links such that they do not protrude past a radially inner or radially outer surface of the links.

Example 158. The assembly of any example herein, particularly any one of examples 152-157, wherein each post comprises a commissure window extending through a thickness of the post.

Example 159. The assembly of any example herein, particularly example 158, wherein the prosthetic device further comprises a valvular structure comprising a plurality of leaflets each including one or more tabs, wherein tabs of adjacent leaflets are disposed within the commissure window to couple the valvular structure to the frame.

Example 160. The assembly of any example herein, particularly any one of examples 152-159, wherein the first member extends from an outflow end portion of the frame toward an inflow end portion of the frame, and wherein the second member extends from the inflow end portion of the frame toward the outflow end portion.

Example 161. The assembly of any example herein, particularly any one of examples 152-160, wherein the first and second side portions are disposed circumferentially on either side of the first member.

Example 162. The assembly of any example herein, particularly any one of examples 152-161, wherein each compliant joint comprises a flexible neck portion configured to deflect circumferentially as the frame moves from the radially compressed state to the radially expanded state.

Example 163. The assembly of any example herein, particularly any one of examples 152-162, wherein the frame comprises three posts.

Example 164. An assembly, comprising:
a prosthetic device comprising:
   a frame that is radially expandable and compressible between a radially compressed state and a radially expanded state, the frame comprising:
      a plurality of posts, one or more posts configured as expansion and locking mechanisms comprising an axially extending first member, an axially extending second member comprising a first side portion and a second side portion defining a channel between them into which at least a portion of the first member extends, and a cap disposed over an outflow end portion of the second member, the cap comprising a biasing member configured to frictionally engage a surface of the first member to allow movement of the first member relative to the second member in a first direction to cause radial expansion of the frame and to prevent movement in a second direction to prevent radial compression of the frame; and
      a plurality of links coupling adjacent posts to one another via one or more compliant joints; and
a delivery apparatus comprising:
   a handle;
   a first actuation member extending from the handle and coupled to an outflow end of the frame, the first actuation member configured to apply a distally directed force to the first member; and
   a second actuation member extending from the handle and coupled to an inflow end of the frame, the second actuation member configured to apply a proximally directed force to the second member;
wherein the prosthetic device is radially expandable from the radially compressed state to the radially expanded state upon application of at least one of the distally directed force and the proximally directed force to the prosthetic device via the first and second actuation members, respectively; and
wherein when the prosthetic device is in the radially expanded state the biasing member engages the first member to prevent compression of the frame.

Example 165. An assembly, comprising:
a prosthetic device comprising:
   a frame that is radially expandable and compressible between a radially compressed state and a radially expanded state, the frame comprising:
      a plurality of posts, one or more posts configured as expansion and locking mechanisms comprising an axially extending first member, an axially extending second member comprising a first side portion and a second side portion defining a channel between them into which at least a portion of the first member extends, and an annular cap disposed over an outflow end portion of the second member, the cap comprising an inner lumen extending along a length of the cap, the inner lumen tapering from a first diameter at an inflow end of the cap to a second, smaller diameter at an outflow end of the cap, and
      a plurality of links coupling adjacent posts to one another via one or more compliant joints; and
a delivery apparatus comprising:
   a handle;
   a first actuation member extending from the handle and coupled to an outflow end of the frame, the first actuation member configured to apply a distally directed force to the first member; and
   a second actuation member extending from the handle and coupled to an inflow end of the frame, the second actuation member configured to apply a proximally directed force to the second member;
wherein the prosthetic device is radially expandable from the radially compressed state to the radially expanded state upon application of at least one of the distally directed force and the proximally directed force to the prosthetic device via the first and second actuation members, respectively; and
wherein when the prosthetic device is in the radially expanded state the first and second side portions engage the first member to prevent compression of the frame.

Example 166. A method, comprising:
inserting a distal end of a delivery apparatus into a vasculature of a patient, the delivery apparatus releasably coupled to a prosthetic device including a radially expandable and compressible frame, the frame comprising:
   a plurality of posts, one or more posts configured as expansion and locking mechanisms comprising an axially extending first member and an axially extending second member comprising a first side portion and a second side portion, the first side portion comprising a locking tooth configured to frictionally engage a surface of the first member to allow movement of the first member relative to the second member in a first direction to cause radial expansion of the frame and to prevent movement in a second direction to prevent radial compression of the frame; and
   a plurality of links configured to couple adjacent posts to one another via one or more compliant joints;
advancing the prosthetic device to a selected implantation site; and
moving at least one of the first member distally and the second member proximally to radially expand the prosthetic device such that the compliant joints deflect circumferentially and such that the locking tooth engages the first member to lock the prosthetic device in a radially expanded state.

Example 167. The method of any example herein, particularly example 166, wherein the first side portion comprises a biasing member configured to bias the locking tooth against the first member.

Example 168. A method, comprising:
inserting a distal end of a delivery apparatus into a vasculature of a patient, the delivery apparatus releasably coupled to a prosthetic device including a radially expandable and compressible frame, the frame comprising:
   a plurality of posts, one or more posts configured as expansion and locking mechanisms comprising an axially extending first member, an axially extending second member comprising a first side portion and a second side portion defining a channel between them into which at least a portion of the first member extends, and a cap disposed over an outflow end portion of the second member, the cap comprising a biasing member configured to frictionally engage a surface of the first member to allow movement of the first member relative to the second member in a first direction to cause radial expansion of the frame and to prevent movement in a second direction to prevent radial compression of the frame; and
   a plurality of links configured to couple adjacent posts to one another via one or more compliant joints;
advancing the prosthetic device to a selected implantation site; and
moving at least one of the first member distally and the second member proximally to radially expand the prosthetic device such that the compliant joints deflect circumferentially and such that the biasing member engages the first member to lock the prosthetic device in a radially expanded state.

Example 169. A method, comprising:
inserting a distal end of a delivery apparatus into a vasculature of a patient, the delivery apparatus releasably coupled to a prosthetic device including a radially expandable and compressible frame, the frame comprising:
   a plurality of posts, one or more posts configured as expansion and locking mechanisms comprising an axially extending first member, an axially extending second member comprising a first side portion and a second side portion defining a channel between them into which at least a portion of the first member extends, and an annular cap disposed over an outflow end portion of the second member, the cap comprising an inner lumen extending along a length of the cap, the inner lumen tapering from a first diameter at an inflow end of the cap to a second, smaller diameter at an outflow end of the cap; and
   a plurality of links configured to couple adjacent posts to one another via one or more compliant joints;
advancing the prosthetic device to a selected implantation site; and
moving at least one of the first member distally and the second member proximally to radially expand the prosthetic device such that the compliant joints deflect circumferentially and such that the first and second side portions engage the first member to lock the prosthetic device in a radially expanded state.

Example 170. The method of any example herein, particularly example 169, further comprising advancing the cap over an outflow end portion of the second member to urge the first and second side portions against the first member.

Example 171. An assembly comprising:
a prosthetic device comprising:
   a radially expandable and compressible frame;
   at least one expansion and locking device that is movable between an elongated position and a retracted position, the expansion and locking device comprising:
      a distal member; and
      a proximal member comprising a locking element configured to prevent the distal member and the proximal member from moving away from one another;
      wherein the distal member and the proximal member are coupled to the frame at axially spaced locations on the frame, wherein the distal member is coupled to the frame at a more distal portion of the frame than the proximal member; and
a delivery apparatus comprising at least one actuation assembly configured to removably couple to the expansion and locking device and to move the distal member and the proximal member towards one another to radially expand the prosthetic device;
   wherein the actuation assembly extends distally past the locking element when the expansion and locking device is in the elongated position and wherein the distal member extends proximally past the locking element when the expansion and locking device is in the retracted position, and wherein the locking element is configured to continuously frictionally engage the actuation assembly when the actuation assembly extends distally past the locking element and is configured to continuously frictionally engage the distal member when the distal member extends proximally past the locking element to continuously lock the expansion and locking device at any position between the elongated position and the retracted position.

Example 172. The assembly of any example herein, particularly example 171, wherein the locking element comprises a spring tooth having a free end that curves in a proximal direction towards a handle of the delivery apparatus.

Example 173. The assembly of any example herein, particularly example 172, wherein the spring tooth exerts a constant holding force on the actuation assembly when the actuation assembly extends distally past the locking element and exerts a constant holding force on the distal member when the distal member extends proximally past the locking element.

Example 174. The assembly of any example herein, particularly any one of examples 171-173, wherein the proximal member comprises a main body having an opening configured to receive the distal member of the expansion and locking device.

Example 175. The assembly of any example herein, particularly example 174, wherein the main body is configured to be coupled to a proximal apex or proximal junction of a frame of the prosthetic device.

Example 176. The assembly of any example herein, particularly any one of examples 171-175, wherein the actuation assembly comprises a first actuation member that is configured to extend to the proximal member or a proximal end of the prosthetic device, and a second actuation member that is removably coupled to the distal member.

Example 177. The assembly of any example herein, particularly example 176, wherein an/the opening of a/the main body of the proximal member is configured to receive the second actuation member of the actuation assembly.

Example 178. The assembly of any example herein, particularly example 176 or example 177, wherein the distal member of the expansion and locking device comprises a first removable coupling member at a proximal end of the distal member and wherein the second actuation member comprises a second removable coupling member at a distal end of the second actuation member, and wherein the first and second removable coupling members are configured to removable couple to one another to removably couple the distal member and the second actuation member.

Example 179. The assembly of any example herein, particularly example 178, wherein the first and second removable coupling members comprise mating threads.

Example 180. The assembly of any example herein, particularly any one of examples 176-179, wherein the first actuation member comprises a sleeve that surrounds the second actuation member, and wherein the second actuation member comprises a pull cable that extends through the sleeve.

Example 181. The assembly of any example herein, particularly example 180, wherein the first actuation member is configured to apply a distally directed force to the proximal member and wherein the second actuation member is configured to apply a proximally directed force to the distal member, wherein the proximally directed force is opposite the distally directed force.

Example 182. The assembly of any example herein, particularly any one of examples 170-181, wherein the distal member comprises a rod that is configured to extend axially towards the proximal member.

Example 183. The assembly of any example herein, particularly example 182, wherein an/the opening of a/the main body of the proximal member is configured to receive the rod of the distal member.

Example 184. The assembly of any example herein, particularly example 182 or example 183, wherein the distal member and a/the second actuation member of the actuation assembly are substantially the same thickness.

Example 185. The assembly of any example herein, particularly any one of examples 170-184, wherein the distal member comprises a main body that is configured to be coupled to a distal apex or distal junction of a/the frame of the prosthetic device.

Example 186. The assembly of any example herein, particularly example 185, wherein a/the rod of the distal member extends axially from the main body.

Example 187. The assembly of any example herein, particularly any one of examples 170-186, wherein the prosthetic device is in a radially compressed position when the expansion and locking device is in the extended position and wherein the prosthetic device is in a radially expanded position when the expansion and locking mechanism is in the retracted position.

Example 188. The assembly of any example herein, particularly example 187, wherein a/the second actuation member of the actuation assembly extends through an/the opening in a/the main body of the proximal member in the extended position and wherein a/the rod of the distal member extends through the opening in the main body of the proximal member in the retracted position.

Example 189. The assembly of any example herein, particularly example 187 or example 188, wherein a/the spring tooth of the proximal member frictionally engages a/the second actuation member of the actuation assembly when the second actuation member is positioned in an/the opening of a/the main body of the proximal member and frictionally engages a/the rod of the distal member when the rod is positioned in the opening to hold the expansion and locking device at any position between the extended position and the retracted position.

Example 190. The assembly of any example herein, particularly example 189, wherein spring tooth permits the rod and second actuation member to move in a first axial direction through the opening relative to the proximal member but prevents the rod and second actuation member from moving in an opposite second axial direction.

Example 191. The assembly of any example herein, particularly example 190, wherein the spring tooth is angled or curved relative to a central longitudinal axis of the opening such that the spring tooth has a concave curvature with respect to the first axial direction.

Example 192. A prosthetic device, comprising:
a radially expandable and compressible frame; and
at least one expansion and locking mechanism coupled to the frame, the expansion and locking mechanism comprising:
   a distal member configured to be removably coupled to an actuation assembly of a delivery apparatus for the prosthetic device; and
   a proximal member comprising a locking element configured to frictionally engage the actuation assembly and the distal member, wherein the locking element is configured to allow movement of the actuation assembly and the distal member relative to the proximal member in a first direction to cause radial expansion of the frame and to prevent movement in a second direction to prevent radial compression of the frame, wherein the proximal member is coupled to a more proximal portion of the frame than the distal member.

Example 193. The prosthetic device of any example herein, particularly example 192, wherein the actuation assembly is configured to be selectively adjusted to move the proximal and distal members towards one another to radially expand the frame.

Example 194. The prosthetic device of any example herein, particularly example 192 or example 193, wherein the locking element is configured to continuously lock the distal member to prevent movement of the distal member in the second direction.

Example 195. The prosthetic device of any example herein, particularly any one of examples 192-194, wherein the locking element comprises a spring tooth that is biased towards an extended position in which the spring tooth frictionally engages the distal member to prevent movement of the distal member in the second direction.

Example 196. The prosthetic device of any example herein, particularly example 195, wherein the spring tooth is angled or curved relative to a central longitudinal axis of a channel of the proximal member such that a distal end of the spring tooth curves or angles in the first direction.

Example 197. The prosthetic device of any example herein, particularly any one of examples 192-196, wherein the distal member comprises a rod that extends axially towards the proximal member, and wherein the proximal member has a/the channel that is configured to receive the actuation assembly and the rod of the distal member.

Example 198. The prosthetic device of any example herein, particularly example 197, wherein the frame is radially expandable and compressible between a radially compressed position and a radially expanded position, and wherein the rod extends proximally through the channel past the locking element in the radially expanded position such that the locking element frictionally engages the rod and not the actuation assembly, and wherein the actuation assembly extends distally through the channel past the locking element in the radially compressed position such that the locking element frictionally engages the actuation assembly and not the rod.

Example 199. The prosthetic device of any example herein, particularly example 198, wherein the rod extends proximally towards, but not to, the proximal member when the frame is in the radially compressed position.

Example 200. The prosthetic device of any example herein, particularly any one of examples 197-199, wherein the rod has a uniform thickness.

Example 201. The prosthetic device of any example herein, particularly any one of examples 197-199, wherein the rod has a non-uniform thickness.

Example 202. The prosthetic device of any example herein, particularly example 201, wherein the rod is tapered such that it is thinner nearer a proximal end of the rod.

Example 203. The prosthetic device of any example herein, particularly any one of examples 182-202, wherein the distal member is coupled to a distal junction or distal apex of the frame.

Example 204. The prosthetic device of any example herein, particularly any one of examples 182-203, wherein the proximal member is coupled to a proximal junction or proximal apex of the frame.

Example 205. The prosthetic device of any example herein, particularly any one of examples 182-204, further comprising a valvular structure comprising one or more leaflets that are configured to regulate the flow of blood through the prosthetic device.

Example 206. An assembly, comprising:
a prosthetic device comprising:
   a frame that is radially expandable and compressible between a radially compressed position and a radially expanded position; and
   at least one expansion and locking mechanism comprising:
      a distal member; and
      a proximal member, the proximal member coupled to the frame at a more proximal portion of the frame than the distal member, the proximal member comprising a locking element configured to allow movement of the distal member relative to the proximal member in a proximal direction to cause radial expansion of the frame and to prevent movement in a distal direction to prevent radial compression of the frame; and
a delivery apparatus comprising:
   a handle;
   a first actuation member configured to apply a distally directed force to the proximal member, wherein the first actuation member extends distally from the handle to the proximal member;
   a second actuation member configured to apply a proximally directed force to the distal member, wherein the second actuation member extends distally from the handle and is removably coupled to the distal member;
   wherein the locking element frictionally engages the second actuation member when the prosthetic device is in the radially compressed position and frictionally engages the distal member when the prosthetic device is in the radially expanded position to continuously lock the prosthetic device at any position between the radially compressed position and the radially expanded position.

Example 207. The assembly of any example herein, particularly example 206, wherein the prosthetic device is radially expandable from the radially compressed position to the radially expanded position upon application of at least one of the distally directed force and the proximally directed force to the prosthetic device via the first and second actuation members, respectively.

Example 208. The assembly of any example herein, particularly example 206 or example 207, wherein the second actuation member extends distally past the locking element when the prosthetic device is in the radially compressed position, and wherein the distal member extends proximally past the locking element when the prosthetic device is in the radially expanded position.

Example 209. The assembly of any example herein, particularly any one of examples 206-208, wherein the locking element comprises a biasing member configured to bias the locking element against the second actuation member when the second actuation member extends over the locking element and configured to bias the locking element against the distal member when the distal member extends over the locking element to only permit movement of the distal member and second actuation member relative to the proximal member in a proximal direction and to continuously prevent movement of the distal member and the second actuation member in an opposite distal direction relative to the proximal member.

Example 210. The assembly of any example herein, particularly any one of examples 206-209, wherein the proximal member comprises a channel that is configured to receive the distal member and the second actuation member, and wherein the locking element extends into the channel to frictionally engage the distal member when the distal member is positioned within the channel and to frictionally engage the second actuation member when the second actuation member is positioned within the channel.

Example 211. The assembly of any example herein, particularly example 210, wherein the locking element is configured to deflect away from a central longitudinal axis of the channel upon application of one or more of the distally directed force and the proximally directed force to permit the distal member and the second actuation member to move proximally relative to the proximal member.

Example 212. The assembly of any example herein, particularly any one of examples 206-211, wherein the distal member comprises a main body that is coupled to a distal apex or distal junction of the frame, and a rod that extends axially from the main body towards the proximal member.

Example 213. The assembly of any example herein, particularly any one of examples 212, wherein the rod is configured to extend through a/the channel of the proximal member.

Example 214. The assembly of any example herein, particularly any one of examples 206-213, wherein the locking elements comprises a spring tooth that is angled or curved relative to a central longitudinal axis of a/the channel of the proximal member such that a distal end of the spring tooth curves or angles in the proximal direction.

Example 215. The assembly of any example herein, particularly any one of examples 206-214, wherein a proximal end of the distal member is removably coupled to a distal end of the second actuation member via a threaded engagement.

Example 216. A method, comprising:
inserting a distal end of a delivery apparatus into a vasculature of a patient, the delivery apparatus releasably coupled to a prosthetic device comprising a radially expandable and compressible frame and an expansion and locking mechanism comprising a distal member and a proximal member;
advancing the prosthetic device to a selected implantation site;
radially expanding the prosthetic device by:
   applying a distally directed force to the proximal member of the expansion and locking mechanism via a first actuation member of the delivery apparatus that is removably coupled to the proximal member; and
   applying a proximally directed force to the distal member of the expansion and locking mechanism via a second actuation member that extends to the proximal member; and
continuously locking the prosthetic device to prevent it from moving towards a more radially compressed position during the entire radial expansion process via a spring tooth included in the proximal member that frictionally engages the second actuation member and the distal member.

Example 217. The method of any example herein, particularly example 216, further comprising decoupling the delivery apparatus from the prosthetic device by decoupling the second actuation member from the distal member of the expansion and locking mechanism.

Example 218. The method of any example herein, particularly example 217, wherein the second actuation member from the distal member comprises unscrewing the second actuation member from the distal member.

Example 219. The method of any example herein, particularly any one of examples 216-218, wherein the applying the proximally directed force comprises pulling the second actuation member.

Example 220. The method of any example herein, particularly any one of examples 216-218, wherein the applying the proximally directed force comprises adjusting a control mechanism included on a handle of the delivery apparatus.

Example 221. The method of any example herein, particularly any one of examples 216-220, wherein the applying the distally directed force comprises pushing a/the handle of the delivery apparatus.

Example 222. The method of any example herein, particularly any one of examples 216-221, wherein the radially expanding the prosthetic device comprises radially expanding the prosthetic device in a series of pulses.

Example 223. The method of any example herein, particularly any one of examples 216-222, wherein the radially expanding the prosthetic device comprises radially expanding the prosthetic device from a radially compressed position to a radially expanded position.

Example 224. The method of any example herein, particularly example 223, further comprising holding the prosthetic device in the radially expanded position via the spring tooth.

Example 225. The method of any example herein, particularly any one of examples 216-224, wherein the spring tooth extends into a channel included in the proximal member of the expansion and locking mechanism, and where the radially expanding the prosthetic device comprises moving the second actuation member proximally through the channel over the spring tooth until the second actuation member clears the spring tooth, and then moving the distal member proximally through the channel over the spring tooth.

Example 226. The method of any example herein, particularly example 225, further comprising frictionally engaging the spring tooth with the second actuation member, and then, once the second actuation member clears the spring tooth, frictionally engaging the spring tooth with the distal member to continuously lock the prosthetic device.

Example 227. A prosthetic device, comprising:
a frame that is radially expandable and compressible between a radially compressed state and a fully radially expanded state, the frame comprising:
   a plurality of posts extending axially from an inflow end of the frame to an outflow end;
   a plurality of links that extend circumferentially between adjacent posts of the plurality of posts; and
   a plurality of compliant joints that each pivotably couples one of the plurality of links to one of the plurality of posts; and
wherein as the frame moves from the radially compressed state to the fully radially expanded state the plurality of compliant joints deflect circumferentially.

Example 228. The prosthetic device of any example herein, particularly example 227, wherein each compliant joint of the plurality of compliant joints comprises a flexible neck portion defining a gap between one of the plurality of posts and one of the plurality of links.

Example 229. The prosthetic device of any example herein, particularly example 228, wherein the gap widens as the frame moves from the radially compressed state to the fully radially expanded state.

Example 230. The prosthetic device of any example herein, particularly examples 228 or example 229, wherein the flexible neck portion of each compliant joint is thinner than and/or narrower than the plurality of links and the plurality of posts.

Example 231. The prosthetic device of any example herein, particularly any one of examples 227-230, wherein one or more of the plurality of compliant joints elastically deform when the frame radially expands from the radially compressed state to a partially expanded state in an initial expansion range of the frame and then plastically deforms when the frame radially expands from the partially expanded state to the fully expanded state in a subsequent expansion range.

Example 232. The prosthetic device of any example herein, particularly any one of examples 227-231, wherein one or more of the plurality of compliant joints comprise a leaf type joint.

Example 233. The prosthetic device of any example herein, particularly any one of examples 227-232, wherein one or more of the plurality of compliant joints comprise a beam type joint.

Example 234. The prosthetic device of any example herein, particularly any one of examples 227-233, wherein one or more of the plurality of compliant joints comprise a flat spring type joint.

Example 235. The prosthetic device of any example herein, particularly any one of examples 227-234, further comprising a plurality of interconnects positioned circumferentially between each pair of adjacent posts of the plurality of posts, wherein each of the plurality of interconnects pivotably couples four of the links.

Example 236. The prosthetic device of any example herein, particularly example 235, wherein one or more of the interconnects, elastically deform when the frame radially expands from the radially compressed state to a/the partially expanded state in an/the initial expansion range of the frame and then plastically deforms when the frame radially expands from the partially expanded state to the fully expanded state in a/the subsequent expansion range.

Example 237. The prosthetic device of any example herein, particularly any one of examples 227-236, wherein each post of the plurality of posts comprises a commissure window extending through a thickness of the post.

Example 238. The prosthetic device of any example herein, particularly any one of examples 227-237, wherein one or more of the plurality of posts are configured as expansion and locking mechanisms and comprise an inner member and one or more outer members.

Example 239. The prosthetic device of any example herein, particularly example 238, wherein the inner member comprises a linear rack having a plurality of teeth arrayed along a length of the inner member, and wherein at least one of the one or more outer members comprises a pawl configured to engage the rack to allow movement of the inner member relative to the outer members in a first direction and prevent movement of the inner member relative to the outer members in a second direction opposite the first direction.

Example 240. A prosthetic device, comprising:
a frame that is radially expandable and compressible between a radially compressed state and a radially expanded state, the frame comprising:
   a plurality of posts, each post comprising an axially extending first member and two axially extending second members, wherein the first member is configured to move axially relative to the second members within a channel of the second members to radially expand and/or radially compress the frame; and
   a plurality of links coupling adjacent posts to one another via one or more compliant joints.

Example 241. The prosthetic device of any example herein, particularly example 240, wherein the frame is formed from a unitary piece of material.

Example 242. The prosthetic device of any example herein, particularly example 240 or example 241, wherein one or more of the plurality of posts comprise a locking mechanism that is configured to allow radial expansion of the frame and prevent radial compression of the frame.

Example 243. The prosthetic device of any example herein, particularly example 242, wherein the locking mechanism comprises at least one locking tooth.

Example 244. The prosthetic device of any example herein, particularly example 242 or example 243, wherein the locking mechanism comprises interlocking teeth on the first member and the second members.

Example 245. The prosthetic device of any example herein, particularly any one of examples 240-244, wherein each link comprises a first end portion and a second end portion and wherein each link is coupled to a post at the first end via a first compliant joint and coupled to an adjacent link at the second portion via a second compliant joint.

Example 246. The prosthetic device of any example herein, particularly example 245, wherein the first compliant joint is oriented toward an inflow end of the frame and the second compliant joint is oriented toward an outflow end of the frame.

Example 247. The prosthetic device of any example herein, particularly example 245 or example 246, wherein each compliant joint comprises a flexible neck portion configured to deflect circumferentially as the frame moves from the radially compressed state to the radially expanded state.

Example 248. The prosthetic device of any example herein, particularly any one of examples 240-247, wherein the first member extends axially from a distal end of the frame towards a proximal end of the frame and wherein the second members extend axially from the proximal end of the frame toward the distal end of the frame.

Example 249. The prosthetic device of any example herein, particularly any one of examples 240-247, wherein the first member extends axially from a proximal end of the frame towards a distal end of the frame and wherein the second members extend axially from the distal end of the frame toward the proximal end of the frame.

Example 250. The prosthetic device of any example herein, particularly example 248 or example 249, wherein the proximal end of the frame is an outflow end of the frame and wherein the distal end of the frame is an inflow end of the frame.

Example 251. The prosthetic device of any example herein, particularly example 248 or example 249, wherein the proximal end of the frame is an inflow end of the frame and wherein the distal end of the frame is an outflow end of the frame.

Example 252. The prosthetic device of any example herein, particularly any one of examples 240-251, wherein the plurality of posts comprise three posts.

Example 253. The prosthetic device of any example herein, particularly any one of examples 240-252, further comprising a plurality of second posts.

Example 254. The prosthetic device of any example herein, particularly example 253, wherein the plurality of second posts extend between an/the proximal end of the frame and a/the distal end of the frame.

Example 255. The prosthetic device of any example herein, particularly example 253 or example 254, wherein the plurality of second posts and the plurality of posts are circumferentially spaced from one another around the frame.

Example 256. The prosthetic device of any example herein, particularly example 255, wherein the plurality of posts and the plurality of second posts are arranged in an alternating order.

Example 257. The prosthetic device of any example herein, particularly any one of examples 253-256, wherein one or more of the plurality of second posts comprise commissure windows that extend through a thickness of the posts.

Example 258. The prosthetic device of any example herein, particular any one of examples 253-257, wherein one or more of the plurality of second posts comprise support cells that are configured to deform when the frame moves between the radially compressed state and the radially expanded state.

Example 259. The prosthetic device of any example herein, particularly example 258, wherein the support cells are partially elastic and are configured to help hold the frame in the radially expanded state.

Example 260. The prosthetic device of any example herein, particularly any one of examples 240-252, wherein the plurality of posts include commissure windows that extend through a thickness of the posts.

Example 261. The prosthetic device of any example herein, further comprising a skirt assembly.

Example 262. The prosthetic device of any example herein, wherein the skirt assembly comprises an inner skirt included and/or mounted on an inner side of the frame and/or an outer skirt included and/or mounted on an outer side of the frame.

Example 263. A prosthetic device, comprising:
a frame that is radially expandable and compressible between a radially compressed state and a radially expanded state, the frame comprising:
   a plurality of links connected by a plurality of compliant joints; and
   the plurality of compliant joints that connect the plurality of links, wherein each compliant joint pivotably couples two of the links; and
wherein as the frame moves from the radially compressed state to the radially expanded state the plurality of compliant joints deflect circumferentially.

Example 264. The prosthetic device of any example herein, particularly example 263, wherein each compliant joint of the plurality of compliant joints comprises a flexible neck portion defining a gap between adjacent links.

Example 265. The prosthetic device of any example herein, particularly example 264, wherein the gap widens as the frame moves from the radially compressed state to the radially expanded state.

Example 266. The prosthetic device of any example herein, particularly examples 263 or example 264, wherein the flexible neck portion of each compliant joint is thinner than and/or narrower than the plurality of links.

Example 267. The prosthetic device of any example herein, particularly any one of examples 263-266, wherein one or more of the plurality of compliant joints elastically deform when the frame radially expands from the radially compressed state to a partially expanded state in an initial expansion range of the frame and then plastically deform when the frame radially expands from the partially expanded state to a fully expanded state in a subsequent expansion range.

Example 268. The prosthetic device of any example herein, particularly any one of examples 263-267, wherein one or more of the plurality of compliant joints comprise a leaf type joint.

Example 269. The prosthetic device of any example herein, particularly any one of examples 263-268, wherein one or more of the plurality of compliant joints comprise a beam type joint.

Example 270. The prosthetic device of any example herein, particularly any one of examples 263-269, wherein one or more of the plurality of compliant joints comprise a flat spring type joint.

Example 271. The prosthetic device of any example herein, particularly any one of examples 263-270, further comprising a plurality of interconnects positioned circumferentially between the compliant joints, wherein each of the plurality of interconnects pivotably couples four of the links.

Example 272. The prosthetic device of any example herein, particularly example 271, wherein one or more of the interconnects, elastically deform when the frame radially expands from the radially compressed state to a/the partially expanded state in an/the initial expansion range of the frame and then plastically deforms when the frame radially expands from the partially expanded state to a/the fully expanded state in a/the subsequent expansion range.

Example 273. The prosthetic device of any example herein, particularly any one of examples 263-272, further comprising an expansion and locking mechanism.

Example 274. The prosthetic device of any example herein, particularly example 273, wherein the expansion and locking mechanism comprises a distal member and a proximal member, wherein the proximal member is coupled to the frame at a more proximal portion of the frame than the distal member, the proximal member comprising a locking element configured to allow movement of the distal member relative to the proximal member in a proximal direction to cause radial expansion of the frame and to prevent movement in a distal direction to prevent radial compression of the frame.

Example 275. The prosthetic device of any of examples 1-274, wherein the prosthetic device is a prosthetic valve.

Example 276. The prosthetic device of example 275, further comprising a plurality of leaflets disposed within the frame for allowing blood to flow through the frame in a first direction and blocking the flow of blood through the frame in a second direction.

Example 277. The prosthetic device of any of examples 275-276, wherein the prosthetic valve is a prosthetic heart valve.

Although the examples herein are primarily directed to prosthetic heart valves, it should be appreciated that the frames of the prosthetic heart valves disclosed herein can be used with other prosthetic devices and/or can be used independently, for example, as stents.

In view of the many possible embodiments to which the principles of the disclosed invention can be applied, it should be recognized that the illustrated embodiments are only preferred examples of the invention and should not be taken as limiting the scope of the invention. Rather, the scope of the invention is defined by the following claims and their equivalents.

1. A prosthetic device, comprising:
a frame that is radially expandable and compressible between a radially compressed state and a fully radially expanded state, the frame comprising:
a plurality of posts extending axially from an inflow end of the frame to an outflow end;
a plurality of links that extend circumferentially between adjacent posts of the plurality of posts; and
a plurality of compliant joints that each pivotably couples one of the plurality of links to one of the plurality of posts; and
wherein as the frame moves from the radially compressed state to the fully radially expanded state the plurality of compliant joints deflect circumferentially.

2. The prosthetic device of claim 1, wherein each compliant joint of the plurality of compliant joints comprises a flexible neck portion defining a gap between one of the plurality of posts and one of the plurality of links.

3. The prosthetic device of claim 2, wherein the gap widens as the frame moves from the radially compressed state to the fully radially expanded state.

4. The prosthetic device of claim 2, wherein the flexible neck portion of each compliant joint is thinner than and/or or narrower than the plurality of links and the plurality of posts.

5. The prosthetic device of claim 1, wherein one or more of the plurality of compliant joints elastically deform when the frame radially expands from the radially compressed state to a partially expanded state in an initial expansion range of the frame and then plastically deforms when the frame radially expands from the partially expanded state to the fully expanded state in a subsequent expansion range.

6. The prosthetic device of claim 1, wherein one or more of the plurality of compliant joints comprise a leaf type joint.

7. The prosthetic device of claim 1, wherein one or more of the plurality of compliant joints comprise a beam type joint.

8. The prosthetic device of claim 1, wherein one or more of the plurality of compliant joints comprise a flat spring type joint.

9. The prosthetic device of claim 1, further comprising a plurality of interconnects positioned circumferentially between each pair of adjacent posts of the plurality of posts, wherein each of the plurality of interconnects pivotably couples four of the links.

10. The prosthetic device of claim 9, wherein one or more of the interconnects, elastically deform when the frame radially expands from the radially compressed state to a partially expanded state in an initial expansion range of the frame and then plastically deforms when the frame radially expands from the partially expanded state to the fully expanded state in a subsequent expansion range.

11. The prosthetic device of claim 1, wherein each post of the plurality of posts comprises a commissure window extending through a thickness of the post.

12. The prosthetic device of claim 1, wherein one or more of the plurality of posts are configured as expansion and locking mechanisms and comprise an inner member and one or more outer members.

13. The prosthetic device of claim 12, wherein the inner member comprises a linear rack having a plurality of teeth arrayed along a length of the inner member, and wherein at least one of the one or more outer members comprises a pawl configured to engage the rack to allow movement of the inner member relative to the outer members in a first direction and prevent movement of the inner member relative to the outer members in a second direction opposite the first direction.

14. A prosthetic device, comprising:
a frame that is radially expandable and compressible between a radially compressed state and a radially expanded state, the frame comprising:
a plurality of posts, each post comprising an axially extending first member and two axially extending second members, wherein the first member is configured to move axially relative to the second members within a channel of the second members to radially expand and radially compress the frame; and
a plurality of links coupling adjacent posts to one another via one or more compliant joints.

15. The prosthetic device of claim 14, wherein the frame is formed from a unitary piece of material.

16. The prosthetic device of claim 14, wherein one or more of the plurality of posts comprise a locking mechanism that is configured to allow radial expansion of the frame and prevent radial compression of the frame.

17. The prosthetic device claim 16, wherein the locking mechanism comprises either a plurality of teeth on the first member and a locking tooth on the second member or a plurality of teeth on the second member and a locking tooth on the first member, wherein the locking tooth is configured to engage the plurality of teeth to allow movement in a first direction to allow radial expansion of the frame and prevent movement in a second direction to prevent radial compression of the frame.

18. The prosthetic device of claim 14, wherein each link comprises a first end portion and a second end portion and wherein each link is coupled to a post at the first end via a first compliant joint and coupled to an adjacent link at the second portion via a second compliant joint.

19. The prosthetic device of claim 18, wherein the first compliant joint is oriented toward an inflow end of the frame and the second compliant joint is oriented toward an outflow end of the frame.

20. The prosthetic device of claim 18, wherein each compliant joint comprises a flexible neck portion configured to deflect circumferentially as the frame moves from the radially compressed state to the radially expanded state.

21. An assembly comprising:
a prosthetic device comprising:
a radially expandable and compressible frame;
at least one expansion and locking device that is movable between an elongated position and a retracted position, the expansion and locking device comprising:
a distal member; and
a proximal member comprising a locking element configured to prevent the distal member and the proximal member from moving away from one another;
wherein the distal member and the proximal member are coupled to the frame at axially spaced locations on the frame, wherein the distal member is coupled to the frame at a more distal portion of the frame than the proximal member; and
a delivery apparatus comprising at least one actuation assembly configured to removably couple to the expansion and locking device and to move the distal member and the proximal member towards one another to radially expand the prosthetic device;
wherein the actuation assembly extends distally past the locking element when the expansion and locking device is in the elongated position and wherein the distal member extends proximally past the locking element when the expansion and locking device is in the retracted position, and wherein the locking element is configured to continuously frictionally engage the actuation assembly when the actuation assembly extends distally past the locking element and is configured to continuously frictionally engage the distal member when the distal member extends proximally past the locking element to continuously lock the expansion and locking device at any position between the elongated position and the retracted position.
